(19) United States
(12) Patent Application Publication
(10) Pub. No.: US 2016/0258005 A1
(43) Pub. Date: Sep. 8, 2016

(54) METHODS FOR RAPID RIBONUCLEIC ACID FLUORESCENCE IN SITU HYBRIDIZATION

(71) Applicant: The Trustees of the University of Pennsylvania, Philadelphia, PA (US)

(72) Inventors: Arjun RAJ, Wynnewood, PA (US); Sydney Shaffer, Philadelphia, PA (US); David Issadore, Philadelphia, PA (US)

(21) Appl. No.: 14/900,494

(22) PCT Filed: Jul. 1, 2014
(86) PCT No.: PCT/US14/45099

§ 371 Date: Dec. 21, 2015

Related U.S. Application Data
(60) Provisional application No. 61/842,201, filed on Jul. 2, 2013.
(60) Provisional application No. 61/938,906, filed on Feb. 12, 2014.

(51) Int. Cl. C12Q 1/68; C12Q 1/70

(52) U.S. Cl. CPC C12Q 1/6827; C12Q 1/6841; C12Q 1/701; C12Q 2600/156

(57) ABSTRACT

The present invention provides a method for improved fluorescent in situ hybridization (FISH) methodology which allows for quantifiable signals to be obtained in a short period of time. In certain embodiments, the method provides for a shorter hybridization time, thereby allowing the present method to be used in screening and rapid diagnostic methods. The present invention also provides a device and reagents for use with the methods of the invention.

### CROSS REFERENCE TO RELATED APPLICATIONS

This application claims priority to and the benefit of U.S. Provisional Application No. 61/842,201, filed Jul. 2, 2013 and 61/938,906, filed Feb. 12, 2014, which are incorporated herein by reference in their entireties.

### STATEMENT REGARDING FEDERALLY SPONSORED RESEARCH OR DEVELOPMENT

This invention was made with government support under Grant No. 1DP2OD008514 awarded by the National Institutes of Health (NIH). The government has certain rights in the invention.

### BACKGROUND OF THE INVENTION

Recently, the emergence of new single cell gene expression measurement techniques have revealed that levels of gene expression can vary hugely from cell to cell (Raj & van Oudenaarden, 2008, Cell 135:216-226; Raj & van Oudenaarden, 2009, Annual Review of Biophysics 38:255-270). These methods include those that are protein-based, such as GFP and immunofluorescence, and those that are nucleic acid based, including single-cell RT-qPCR (Bengtsson et al., 2005, Genome Res 15:1388-1392; Bengtsson et al., 2008, BMC Mol. Biol. 9:63), digital RT-PCR (Warren et al., 2006, Proc Natl Acad Sci USA 103:17807-17812), single-cell sequencing (Tang et al., 2009, Nat Methods 6:377-382) and single molecule RNA fluorescence in situ hybridization (single molecule RNA FISH).

RNA FISII offers a number of advantages over other single cell expression quantification tools. It offers the ability to detect individual RNA molecules via fluorescence microscopy, in which each RNA molecule appears in the cell as a bright, diffraction limited spot (Femino et al., 1998, Science 280:585-590; Raj et al., 2008, Nat Methods 5:877-879). Using software to count the spots, the absolute number of RNA in individual cells can be quantified without requiring any amplification. Moreover, spot positions can be analyzed to gain insights into the location of RNA within the cell, which is often useful in developmental systems in which different cells express different genes (Raj et al., 2010, Nature 463:913-918), or in situations in which the subcellular location of the RNA can reveal new biology (Levesque & Raj, 2013, Nat Methods, doi:10.1038/nmeth.2372; Maamar et al., 2013, Genes & development (2013), doi:10.1101/gad.217018.113).

RNA FISH does, however, suffer from some important drawbacks compared to other methods. One is that it is typically a low-throughput method in the sense that, like RT-qPCR, only around five or so genes can typically be analyzed at a time, although barcoding schemes can increase this number to many dozens and potentially hundreds (Lubeck & Cai, 2012, Nat Methods 9:743-748). Yet another issue is that most current protocols rely on a long hybridization (often overnight), and series of washes in order to generate adequate and specific signals. The latter limitation hinders the use of RNA FISH in many scenarios, as it is considerably slower than RT-qPCR in practice, which usually takes on the order of hours to complete. The lack of a rapid version of RNA FISH also places severe restrictions on its use in diagnostic applications, in which timely results are hugely important.

Influenza viruses are a major source of respiratory illness, causing significant morbidity and mortality throughout the world. In the United States, between 5-20% of the population contracts influenza each year, resulting in up to 49,000 influenza-associated deaths (Centers for Disease Control and Prevention. Morbidity and mortality weekly report. 2010; 59(33):1057-62) and the loss of $87.1 billion in economic output (Molinari et al., Vaccine. 2007; 25(27): 5086-96). Moreover, the segmented nature of the viral genome leads to high levels of variability facilitated by viral reservoirs in aquatic birds, poultry and swine, leading to periodic outbreaks of new strains with potentially devastating consequences (Cox et al., Vaccine. 2003; 21(16):1801-3). Thus, strategies to effectively counter the impact of influenza are a major goal of public health.

One focus of these efforts is vaccination. Influenza vaccines are effective, but difficulties associated with producing the vaccine (typically in chicken eggs) means that vaccines can be in short supply, and others remain unvaccinated for other reasons such as allergies to the vaccine. Also, the high mutability of the virus requires that the influenza vaccines must be reformulated and re-administered on a regular basis.

Anti-viral treatments are the other means by which to mitigate the effects of influenza infections. Currently, the available treatments include oseltamivir and zanamivir (Fiore et al., Morbidity and mortality weekly report. 2011; 60(1): 1-24), which can reduce the duration of influenza infections by 30% (Dharan et al. JAMA. 2009; 301(10): 1034-41) by inhibiting the release of new viral particles from infected cells (Fiore et al. Morbidity and mortality weekly report. 2011; 60(1): 1-24). These anti-virals have relatively few side effects, and so their prescription is strongly indicated in confirmed cases of influenza (Fiore et al. Morbidity and mortality weekly report. 2011; 60(1): 1-24). However, these medications are expensive, available only in limited quantities, and indicated for use against certain subtypes only (Dharan et al. JAMA. 2009; 301(10): 1034-41; Bloom et al., Science. 2010; 328(5983): 1272-5; Mai et al., New England Journal of Medicine. 2010; 362(1): 86-7). Moreover, in 2007-2008, a strain of influenza resistant to anti-virals was in circulation, and while that strain is less prevalent currently, it may reemerge at any time (Fiore et al. Morbidity and mortality weekly report. 2011; 60(1): 1-24; Dharan et al. JAMA. 2009; 301(10): 1034-41). Conversely, clinicians will sometimes prescribe antibiotics to patients who actually have influenza, and this inappropriate use of antibiotics contributes to the development of drug-resistant bacteria. For these reasons, any diagnostic that would enable clinicians to limit anti-viral administration strictly to those who could benefit from it would be very valuable. Furthermore, anti-virals are most effective when administered within the first 12-48 hours of the onset of symptoms (Aoki et al. J. Antimicrob. Chemother. 2003; 51(1): 123-9). Thus, this imposes strict time constraints for a diagnostic to provide relevant information to the clinician that may alter patient care.

The current gold standard for influenza detection are assays based upon RT-PCR detection of influenza RNA in nasal aspirates or epithelial swabs. While these assays are highly sensitive (typically detecting roughly 92.3% of cases (Li et al. J. Infect. 2012; 65(1):60-3); FIG. 7), they usually take at least 3 hours to run (Van Wesenbeeck et al., J. Clin. Microbiol. 2013; 51(9): 2977-85), often in a dedicated viral pathology lab, and typically in a multiwell format that processes several samples in batch, meaning that one must wait until enough samples are in hand before running the assay. More recently, faster RT-PCR-based assays that run in around 80 minutes have appeared, but these also suffer from relatively poor sensitivity (<80%) (Li et al. J. Infect. 2012; 65(1):60-3). In addition to the time required to run the assay, there is time overhead involved in getting samples to a clinical lab and tracking down patients once the results come back, making it likely that that the window for treatment with anti-virals will likely already have closed. Thus, many clinicians opt not to test for influenza infection at all. Also, delays in diagnosis prevent the rapid implementation of protocols to prevent the further spread of infection.

For these reasons, there is a clear need for rapid, sensitive and specific diagnostics for influenza infections, especially ones that can be administered at or very near the point of care within the timeframe of a doctor's visit. Several such diagnostics are currently available, mostly utilizing antibodies targeting the NP protein (Chomel et al., J. Virol. Methods. 1989; 25(1): 81-91) in enzyme-linked immunosorbent assays (ELISAs) (FIG. 7). While these assays are relatively rapid (10-15 minutes (Chartrand et al., Future Microbiol. 2010; 5(10): 1451-5; Centers for Disease Control and Prevention. CDC—Seasonal Influenza (Flu)—Rapid Diagnostic Testing for Influenza: Information for Health Care Professionals [Internet; cited 2013 Oct. 8]. http://www.cdc.gov/flu/professionals/diagnosis/rapidclin.htm.)) and can improve patient outcome considerably (Barenfanger et al., J. Clin. Microbiol. 2000; 38(8): 2824-8), they suffer from a number of drawbacks. The main flaw is that they tend to have very poor sensitivity, only detecting around 50-70% of influenza infections (Chartrand et al., Ann Intern Med. 2012; 156(7): 500-11) (FIG. 7). Newer immunofluorescence-based assays have somewhat better sensitivity, but still remain below 80% detection rate (Lewandrowski et al., Am. J. Clin. Pathol. 2013; 139(5):684-9). At this rate of detection, many clinicians again opt not to perform the diagnostic assay. The other issue with antibody based assays is that the production of antibodies is expensive and slow, and it is difficult and sometimes not possible to produce antibodies specific to particular variants. This means that it is difficult to rapidly ramp up production of antibody-based assays that can detect and discriminate new strains that come into circulation. Such specificity is important because of the periodic appearance of sudden outbreaks of new influenza strains (such as H1N1 in 2009), each of which may require a different course of clinical action (Faix et al., N Engl J Med. 2009 Aug. 13; 361(7):728-9).

Thus, there is a need in the art for methods and devices that can obtain quantifiable single molecule RNA FISH signals in a relatively short period of time. The present invention addresses these unmet needs.

### SUMMARY OF THE INVENTION

The invention includes a method for improved fluorescent in situ hybridization (FISH) methodology which allows for quantifiable signals to be obtained in a short period of time.

The invention includes a method for rapid detection of a target nucleic acid in a sample. The method comprises contacting the sample with a non-crosslinking fixative, thereby producing a fixed sample, contacting the fixed sample with a hybridization solution, the hybridization solution comprising at least one labeled probe which hybridizes to a region of the target nucleic acid, wherein the detection of the labeled probe indicates the detection of the target nucleic acid in the sample, and wherein the detection method is conducted in less than 5 hours.

The invention further includes a method for a rapid detection of an influenza target nucleic acid in a cell from a biological sample. The method comprises contacting a cell of the sample with a non-crosslinking fixative, thereby producing a fixed cell, contacting the fixed cell with a hybridization solution, the hybridization solution comprising at least one labeled probe which hybridizes to a region of the influenza target nucleic acid, wherein the detection of the labeled probe indicates the detection of the influenza target nucleic acid in the cell, and wherein the detection method is conducted in less than 5 hours.

The invention additionally includes a fluidic device for detection of influenza in a sample. The fluidic device comprises one or more openings in fluid communication with one or more liquid reservoirs or wells, wherein the liquid reservoirs or wells comprise one or more labeled probes depicted in FIGS. 23, 24, 35, and 26 (SEQ ID NOs: 1-961).

The invention further includes a kit comprising at set of probes for detection of nucleic acids in a sample and instructions for use thereof.

In one embodiment, the non-crosslinking fixative comprises an alcohol selected from the group consisting of ethanol and methanol. In some embodiments, the target nucleic acid is RNA. In an additional embodiment, the RNA is selected from the group consisting of messenger RNA, intronic RNA, exonic RNA, and non-coding RNA. In further embodiments, the target nucleic acid comprises a mutational variant. In one embodiment, the one labeled probe concentration is about 0.1 mM to about 20 mM. In another embodiment, the one labeled probe concentration is about 3 mM to about 4 mM. In certain embodiments, the sample is contacted with the non-crosslinking fixative for about 10 minutes. In yet other embodiments, the sample is contacted with the non-crosslinking fixative for about 2 minutes. In some aspects, the fixed sample is contacted with the hybridization solution for less than about 2 hours. In further aspects, the fixed sample is contacted with the hybridization solution for less than about 1 hour. In further aspects, the fixed sample is contacted with the hybridization solution for less than about 10 minutes. In yet further aspects, the fixed sample is contacted with the hybridization solution for less than about 1 minute. In other aspects, the fixed sample is contacted with the hybridization solution for less than about 30 seconds. In one embodiment, the detection method is conducted in less than 2 hours. In another embodiment, the detection method is conducted in less than 10 minutes. In yet another embodiment, the detection method is conducted in less than 5 minutes. In one aspect, at least one probe is labeled with a fluorophore. In another aspect, the method comprises detecting the fluorophore. In a further aspect, the method is used to quantify the presence of the target nucleic acid. In yet another aspect, the method is used to investigate chromosome structure and transcriptional activity. In certain aspects, the method of is used to identify a mutation in the target nucleic acid. In some aspects, the method is used in high-throughput screening. In additional aspects, the method is used in high-throughput screening. In another aspect, the method is used in rapid diagnostics. In one embodiment, the target nucleic acid comprises an viral nucleic acid sequence. In other embodiments, the viral nucleic acid comprises an influenza nucleic acid sequence. In yet another embodiment, the probe is one or more oligonucleotides depicted in FIGS. 23, 24, 35, and 26 (SEQ ID NOs: 1-961).

In one embodiment, the method comprises the use of a microfluidics device, the microfluidics device comprising one or more openings in fluid communication with one or more liquid reservoirs or wells. In another embodiment, the microfluidics device is optically transparent. In a further embodiment, the sample is introduced into a liquid reservoir or well of the microfluidics device. In yet a further embodiment, the method comprises introducing a fluid into the liquid reservoir or well of a microfluidics device. In certain embodiments, the fluid comprises one or more of a labeled probe, a non-crosslinking fixative, or a buffer. In other embodiments, the microfluidics device comprises the labeled probe preloaded into a liquid reservoir or well.

In one embodiment, the sample of this invention is obtained from a mammal. In a further embodiment, the mammal is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A through 1E, is a diagram and set of images showing the RNA FISH scheme and demonstration of rapid hybridization. FIG. 1A: Schematic of the single molecule RNA FISH method, in which dozens of short fluorescently labeled oligonucleotides that all target the same RNA molecule are used. FIG. 1B: Image showing RNA FISH targeting mRNA from the TBCB gene under standard overnight hybridization conditions (formaldehyde fixation). Each spot is a single mRNA molecule. FIG. 1C: Image showing RNA FISH signals from an attempt at rapid hybridization with a high concentration of probe but with formaldehyde fixation. FIGS. 1D and 1E: Traditional overnight hybridization and Turbo RNA FISH hybridization using methanol-fixed cells. All images are maximum projections of a stack of optical sections encompassing the three-dimensional volume of the cell.

FIG. 2, comprising FIGS. 2A and 2B, is a set of graphs showing a comparison of fixation conditions for both traditional overnight hybridizations and rapid hybridization. FIG. 2A: Comparison of number of spots detected for the TBCB gene with probes labeled with the Alexa 594 fluorophore. FIG. 2B: Comparison of number of spots detected for the TOP2A gene with probes labeled with the Cy3 fluorophore.

FIG. 3, comprising FIGS. 3A through 3C, is a set of graphs showing quantification of signal quality and comparison of different hybridization times and probe concentrations. FIG. 3A: Schematic depicting the manner in which signal quality is quantified via threshold sensitivity. FIG. 3B: Sensitivity of threshold measured in varying probe concentrations and hybridization times. The dotted line represents the sensitivity of a traditional overnight RNA FISH. Error bars reflect standard error of the mean. FIG. 3C: Spot counts for the same conditions as in FIG. 3B. Error bars reflect standard deviation.

FIG. 4, comprising FIGS. 4A and 4B, is a set of graphs showing a comparison of RNA FISH signal from Turbo RNA FISH (5 minutes) to the traditional RNA FISH protocol performed with a variety of hybridization times. FIG. 4A: Comparison of RNA FISH signal sensitivity. Error bars reflect standard error of the mean. FIG. 4B: Comparison of RNA FISH spot count. Error bars reflect standard deviation.

FIG. 5 is a set of images depicting the demonstration of Turbo iceFISH. Turbo FISH was performed using iceFISH probes that targeted a total of 20 introns in genes on chromosome 19 (right panels), while simultaneously performing RNA FISH for TOP2A mRNA (left panels). Turbo FISH was compared to conventional RNA FISH performed overnight (top vs. bottom panels). All images are maximum projections of a stack of optical sections encompassing the three-dimensional volume of the cell. DAPI (nuclear stain) is in blue.

FIG. 6, comprising FIGS. 6A and 6B, depict the results of experiments demonstrating Turbo SNP FISH. FIG. 6A: Demonstration of SNP FISH efficacy under Turbo FISH and conventional RNA FISH conditions in WM983b cells. BRAF mRNA was targeted with guide probes, and then used detection probes that targeted either the V600E mutation for which BRAF is heterozygous in this cell line (top panels) or a common region for which BRAF is homozygous in this cell line (bottom panels). Left panels show the signals from the guide probe (that labels the mRNA), the middle panel shows the detection probe that detects the wild-type sequence, and the right panel shows the detection probe that detects the mutant sequence. FIG. 6B: Quantification of RNA as being either mutant or wild-type in this cell-line. Each bar corresponds to data from a single cell.

FIG. 7 is a graph depicting that current influenza diagnostics have long assay times or poor sensitivity. Rapid influenza diagnostics suffer from poor sensitivity; RT-PCR assays have high sensitivity, but run for 4 hours. An assay according to the invention (e.g., Flu Turbo FISH) has an assay time less than about 5 minutes (e.g., 3 min.) and a high sensitivity greater than 90% (e.g., approaching 100%).

FIG. 8 depicts that tiling of labeled single-stranded DNA oligonucleotides targeting influenza RNA provided rapid detection of influenza in situ. A set of ssDNA oligonucleotides targeting influenza were designed that yielded bright signal via microscopy. Decreasing the number of infected cells in the sample led to fewer positive cells of similar intensity. The high signal allowed to the detection of cells with a 20× air objective. Cell nuclei were stained with DAM.

FIG. 9 depicts rapid RNA FISH detection of influenza virus. MDCK cells were infected with influenza and subjected to ultra-rapid RNA FISH at the times indicated. Rapid RNA FISH (5 min.) was also performed on uninfected cells as a control (far right panel). Signals of similar intensity were observed even after 10 seconds of hybridization. Cell nuclei were stained with DAPI.

FIG. 10 are images depicting RNA FISH signals from probes targeting different influenza viral mRNA. MDCK cells were infected with influenza and oligonucleotide probe pools specific to each viral mRNA were used. Each viral mRNA was transcribed from a particular viral genome segment, as indicated on the micrographs (Segments 1-8). Some showed nuclear localization, cytoplasmic localization, or both. Images were acquired using 5 minute hybridizations and 100× magnification. Cell nuclei were stained with DAPI.

FIG. 11 depicts detection of viral genomic and messenger RNA in the same cells. Probe sets were designed that target genomic RNA and messenger RNA then labeled the oligonucleotides with different fluorescent dyes. The genomic RNA and messenger RNA display different spatial patterns and expression levels in individual cells.

FIG. 12, comprising FIGS. 12A through 12C, show computational analysis of images and determination of specificity/sensitivity of computational image analysis. FIG. 12A: Individual cells were detected computationally using DAPI. FIG. 12B: Fluorescence intensity was measured in the cells detected computationally. FIG. 12C: A receiver-operator curve determines how well positives were discriminated from negatives as detection threshold is varied. A working assay minimizes false positives but has a high rate of true positives for a particular threshold. This analysis determines the appropriate threshold to optimize sensitivity and specificity.

FIG. 13 depicts the detection of influenza subtypes A and B with high specificity by RNA FISH. Probes targeting influenza A bind with high affinity and produced high intensity signal in influenza A infected MDCK cells and produce no detectable signal in influenza B infected cells. Similarly, influenza B RNA FISH probes target only influenza B infected cells. Cell nuclei were stained with DAPI.

FIG. 14 depicts discrimination of cells co-infected with influenza A and B at low magnification by ultra-rapid RNA FISH targeting the different subtypes. MDCK cells were exposed to influenza A and B. Individual cells were infected with either influenza A or influenza B, or were co-infected with both subtypes. Influenza A infected cells (red) can be distinguished from influenza B infected cells (green) using both a 20× objective and 100× objective. Cell nuclei were stained with DAPI. The results of this experiment demonstrates the ability of ultra-rapid RNA FISH to discriminate between different subtypes of influenza.

FIG. 15 depicts the detection of influenza H1N1, H3N2, and influenza B with high specificity by RNA FISH. Probes targeting H1N1, H3N2, and influenza B hind with high affinity and produce high intensity signal in MDCK cells infected with H1N1, H3N2, and influenza B, respectively. Additionally, probes targeting H1N1, H3N2, and influenza B produce no detectable signal in cells infected with other influenza sub-types. Cell nuclei were stained with DAPI.

FIG. 16 depicts the specific detection of influenza types and subtypes in a single assay. Probe sets were designed that are specific to the H1N1 and H3N2 strains of influenza A and also influenza B, having oligonucleotides that exhibited at least 6 base mismatches with the other cross-targets. Labeling the same cells with all three probes at once demonstrates that the probes are highly specific.

FIG. 17, comprising FIGS. 17A and 17B, depicts discrimination of single base differences using masked probes (SEQ ID NOs: 449-451). FIG. 17A: when using a regular 20-30mer oligonucleotide, the free energy differences between a perfect match and a mismatch are relatively small. However, using a masked probe, only a shorter toehold region is available for binding, providing much more specificity. FIG. 17B: use of the masked probe design differentiates wild-type influenza from the A/California/07/2009 strain having a 823 C>T mutation (SEQ ID NOs: 447-451). A “guide” probe targeting common regions of the neuraminidase (NA) gene was used to demonstrate the presence of influenza infection. The probes detect the appropriate targets with virtually no cross-hybridization. Images were obtained with a 100× objective. Cell nuclei were stained with DAPI.

FIG. 18, comprising FIGS. 18A through 18E, depicts a robust fluidic device for automating the RNA FISH assay. FIG. 18A: one embodiment of the fluidic device of the invention is depicted. The fluidic device traps cells under a filter, holding them in place for imaging on an inverted microscope. Hybridization and wash solution are flowed over the cells and the entire assay may be performed within the fluidic device. Thus, the assay using the fluidic device may be automated. FIG. 18B: a prototype of the device is depicted. Briefly, he fluidic device traps cells under a filter, holding them in place for imaging on an inverted microscope. Hybridization and wash solutions are flowed over the cells to carry out the entire assay within the fluidic device, thereby automating the procedure. FIG. 18C: the prototype device was used to capture infected cultured MDCK cells, perform rapid RNA FISH, and image the results using a low power 20× objective. An increase in signal was observed in the infected cells. Cell nuclei were stained with DAPI. FIG. 18D: the prototype test device was used on uninfected cultured MDCK cells. No signal was observed in the uninfected cells. Cell nuclei were stained with DAPI. FIG. 18E: Construction of a robust fluidic device automating the RNA FISH assay.

FIG. 19 depicts single molecule RNA FISH using an adapted Turbo FISH protocol on the microfluidic device platform.

FIG. 20: Demonstrates that RNA FISH for influenza is highly sensitive. To test the sensitivity of the device, cells were infected with decreasing viral titers and rare infected cells were easily detected.

FIG. 21: Detection of RNA in cells from a nasal swab. A nasal swab was obtained from a healthy adult. The cells from the swab were run through a prototype fluidic device and rapid RNA FISH was performed on the cells, targeting the Lamin A/C (LMNA) mRNA. Single mRNA molecules were detected in this sample, indicating the ability to detect RNA in samples similar to those obtained in the clinic.

FIG. 22, comprising FIGS. 22A through 22C, depicts devices having multiple wells for multiplex RNA FISH. Multiple RNA FISH samples can be simultaneously run by splitting the sample flow into multiple wells. A filter in each well captures cells for interrogation by different sets of RNA FISH probes, each with a different target. FIG. 22A: A multiplex fluidic device in which 4 assays can be run simultaneously by splitting the sample between 4 different capture areas. Top view of the multiplex fluidic device (left panel). Side view of the multiplex fluidic device connected to tubing (right panel). FIG. 22B: Multiple layers of laser micromachined laminate sheets were assembled to construct the multiplex microfluidic device in FIG. 22A. FIG. 22C: A multiplex fluidic device in which 16 assays can be run simultaneously by splitting the sample between 16 different capture areas (left panel); schematic of “shower head” geometry (right panel).

FIG. 23 depicts probe sets used to detect eight segments of influenza virus (PR8), (SEQ ID NOs: 1-336).

FIG. 24 depicts probe sets for distinguishing influenza A and B (HA and NA segments), (SEQ ID NOs: 337-400).

FIG. 25 depicts probe sets for influenza single nucleotide polymorphism (SNP) FISH detection, (SEQ ID NOs: 401-451).

FIG. 26 depicts probe sets for distinguishing genomic and mRNA for influenza subtypes A/California/07/2009, A/Texas/50/2012, and B/Brisbane/60/2008, (SEQ ID NOs: 452-961).

### DETAILED DESCRIPTION

The present invention provides an improved fluorescence in situ hybridization (FISH) method for detecting one or more target nucleic acids. The method provides for a faster FISH methodology by shortening the amount of time necessary for efficient hybridization. The present method thereby allows for FISH to be used in high-throughput screening methods and rapid diagnostics.

In a specific embodiment, the methods of the invention can be used for influenza detection. As applicants have discovered, detection of influenza can be significantly decreased and high specificity can be maintained. As described herein probe concentration and fixation conditions were found that increased the speed of the assay by 100×, so detection time is under 5 minutes, sometimes as little as 10 seconds. Select specific probes for RNA FISH that result in high specificity were identified from a set of 336 single-stranded DNA oligonucleotide probes collectively covering all 8 segments of the influenza viral mRNA. High sensitivity was demonstrated, as the probes brightly illuminated cells infected with influenza virus and generated little to no signal from uninfected cells. As the level of infection was lowered, the signal intensity remained high enough that a 20× objective instead of a high-sensitivity 100× objective. The ability to use a lower magnification microscopy is advantageous for designing a simple instrument for reading the output. Applicants have also combined the RNA FISH technique with microfluidic devices which enables cheap and effective processing of clinical samples suitable for point of care diagnostics.

Thus, the invention provides a viable rapid diagnostic for influenza infection. The advantages over current assays are speed, sensitivity, and specificity. This methodology also has many applications beyond influenza detection as well. Further applications could involve simultaneous detection of other viruses, such as a respiratory viral panel. Other applications include detection of expression of particular genes in cells (e.g., circulating tumor cells).

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±20% or ±10%, ±5%, ±1%, or ±0.1% from the specified value, as such variations are appropriate.

The term “mutation” as used herein refers to any change of one or more nucleotides in a nucleotide sequence.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 75% homology.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

A “portion” of a polynucleotide means at least at least about five to about fifty sequential nucleotide residues of the polynucleotide. It is understood that a portion of a polynucleotide may include every nucleotide residue of the polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Naturally occurring” as used herein describes a composition that can be found in nature as distinct from being artificially produced. For example, a nucleotide sequence present in an organism, which can be isolated from a source in nature and which has not been intentionally modified by a person in the laboratory, is naturally occurring.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The terms “patient,” “subject,” “individual,” and the like are used interchangeably herein, and refer to any animal, or cells thereof whether in vitro or in situ, amenable to the methods described herein. Preferably, the patient, subject or individual is a mammal, and more preferable, a human.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

By “microfabrication” is meant to refer to a set of techniques used for fabrication of micro- or nanostructures. In certain embodiments, microfabrication includes, but is not limited only to, the following techniques: photolithography, electron beam lithography, laser ablation, direct optical writing, thin film deposition (spin-coating, spray coating, chemical vapor deposition, physical vapor deposition, sputtering), thin film removal (development, dry etching, wet etching), replica molding (soft lithography), embossing, forming or bonding.

By “microchannel” is meant to refer to a tube with nano- or microscopic cross-section. In certain embodiments, a microchannel or channel has a size in the range of 0.1-200 pm. In other embodiments, of the present invention, microchannels are fabricated into microfluidic devices by means of microfabrication.

By “macrochannel” is meant to refer to a tube of size larger than a microchannel (>200 pm)

By “channel” is meant to refer to either a microchannel or a macrochannel.

By “microfluidic device” is meant to refer to the microfabricated device comprising microchannels or circuits of microchannels, which are used to handle and move fluids. Preferably, microfluidic devices can include components like junctions, reservoirs, valves, pumps, mixers, filters, chromatographic columns, electrodes, waveguides, sensors etc. Microfluidic devices can be made of polymer (e.g., PDMS, PMMA, PTFE, PE, epoxy resins, thermosetting polymers), amorphous (e.g., glass), crystalline (e.g., silicon, silicon dioxide) or metallic (e.g., Al, Cu, Au, Ag, alloys) materials. In certain embodiments, a microfluidic device can contain composite materials or can be a composite material. The microfluidic pipette is a microfluidic device.

By “well” is meant to refer to a part of the device, which is a solution reservoir for reagents.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to an improvement to the RNA FISH method, which currently relies upon a long hybridization reaction and a series of washes to generate adequate signals. The present invention addresses the unmet need for rapid hybridization by providing a method to obtain quantifiable single molecule RNA FISH signals in a short time period, for example, in certain embodiments, 5 minutes or less as opposed the long incubation hours (12-16 h) in the current state of the art. The present invention relates to the discovery that the use of a non-crosslinking fixative allows for shorter hybridization times, while generating images equivalent to those using formaldehyde fixed cells obtained with overnight hybridization. In certain embodiments, the method comprises administering to a sample a hybridization solution comprising an increased concentration of probes, for example, in certain embodiments, up to 100× compared to standard methods.

The present invention, in certain instances referred to herein as “Turbo FISH”, increases the throughput of FISH methodology. In certain embodiments, the present method shortens the time necessary to obtain results, thereby making FISH methods more applicable to point-of-care and rapid diagnostics.

The present invention may be utilized in any RNA FISH application known in the art. For example, the present invention may be used in methods to detect the presence of a target sequence, the location of a target sequence, the amount of a target sequence, the amount of gene expression, chromosomal structure, the presence of a mutation, and the like. For example, in one embodiment, the method is used for iceFISH (intron chromosomal expression FISH), which targets introns that reveals chromosome structure and transcriptional activity. In another embodiment, the method is used for SNP FISH to detect single nucleotide differences in individual transcripts.

The present method may be utilized in diagnostic, prognostic, and screening methods. For example, in certain embodiments, the method is used to detect the presence, location, or amount one or more biomarker associated with a disease or disorder. In another embodiment, the present method is used in a high-throughput screening method to determine an effect of a test compound.

### RNA FISH Method

In situ hybridization (ISH) is a method that utilizes nucleic acid probes to detect DNA or RNA targets in cells via Watson-Crick base pairing of the probe to the target. A version of fluorescence-based ISH targeting RNA (RNA FISH) in which tens of fluorescently-labeled DNA oligonucleotide probes are used, each of which bind to different segments of the same RNA target, has been previously described (Raj et al., 2008, Nat Methods 5:877-879) and is incorporated by reference herein in its entirety. The method in Raj leads to a signal concentration at the target RNA, appearing as a bright spot in a fluorescence microscope.

The present invention includes use of a highly sensitive, yet fast RNA FISH method to identify mutations in a targeted nucleic acid sequence. Additional description and explanation of RNA FISH methodologies can be found in copending patent application publication numbers WO/2010/030818, WO/2012/106711, and U.S. Provisional Patent Application No. 61/785,498, the entire contents of each are incorporated by reference herein it their entirety.

Probes useful in this invention may be DNA, RNA or mixtures of DNA and RNA. They may include non-natural nucleotides, and they may include non-natural internucleotide linkages. Non-natural nucleotides that increase the binding affinity of probes include 2′-O-methyl ribonucleotides, for example. The lengths of probes useful in this invention can be about 15-40 nucleotides for typical DNA or RNA probes of average binding affinity. Preferred lengths of DNA probes and RNA probes are in the range of about 15-20 nucleotides, more preferably 17-25 nucleotides and even more preferably 17-22 nucleotides. In certain embodiments, the probes are about 20 nucleotides long. If means are included to increase a probe's binding affinity, the probe can be shorter, as short as seven nucleotides, as persons in the art will appreciate. A fluorophore can be attached to a probe at any position, including, without limitation, attaching a fluorophore to one end of a probe, preferably to the 3′ end. The probes may be included in a hybridization solution that contains the probes in excess.

The probes may be designed to specifically bind to any target nucleic acid, including RNA, mRNA, microRNA, siRNA, and the like. In certain embodiments, a probe specifically binds to a mutational variant of the nucleic acid, including, for example, a single nucleotide variant.

In certain embodiments, more than one type of probe is used. For example, in certain embodiments, about 1-1000 different probes are used. In one embodiment, each of the different probes are labeled with a similar of different fluorophore are hybridized simultaneously to a target sequence of a nucleotide molecule, such as an RNA molecule. In certain embodiments, the number of probes can range from 4-100, from 10-80, from 15-70, or from 20-60. A fluorescent spot is created that can be detected from the combined fluorescence of the multiple probes. The probes can be non-overlapping, meaning that the region of the target sequence to which each probe hybridizes is unique (or at least non-overlapping). Probes in a set of 2 or more for a selected target sequence can be designed to hybridize adjacently to one another or to hybridize non-adjacently, with stretches of the target sequence, from one nucleotide to a hundred nucleotides or more, not complementary to any of the probes.

A single cell can be probed simultaneously for multiple RNA target sequences, either more than one target sequence of one RNA molecule, or one or more sequences of different RNA molecules. Additionally, one target sequence of an RNA molecule can be probed with more than one set of probes, wherein each set is labeled with a distinguishable fluorophore, and the fluorophores are distinguishable. In one embodiment, the guide probe and the detection probe will have distinguishable fluorophores. Using more than one color for each of multiple targets permits the use of color-coding schemes in highly multiplexed probing methods, according to the present invention.

Methods of the present invention may also include determining if one or more spots representing a target sequence is present. Methods according to the present invention also include counting spots of a given color corresponding to a given RNA species. When it is desired to detect more than one RNA species, different sets of probes labeled with distinct fluorophores can be used in the same hybridization mixture.

Spots can be detected utilizing microscopic methods. A confocal microscope, or a wide-field fluorescence microscope is sufficient. There is no limitation to the type of microscope used.

In one embodiment, the present invention provides a kit, generally comprising a set of probes, solutions, fixatives, an instruction manual for performing any of the methods contemplated herein, and optionally the computer-readable media as described herein.

### Target Nucleic Acid Sample

As contemplated herein, the present invention may be used in the analysis of sample for which nucleic acid analysis may be applied, as would be understood by those having ordinary skill in the art. For example, in one embodiment, the sample comprises at least one target nucleic acid, whose presence, location, or amount is desired to be investigated. For example, in certain embodiments, the nucleic acid can be mRNA. However, it should be appreciated that there is no limitation to the type of nucleic acid sample, which may include without limitation, any type of RNA, cDNA, gnomic DNA, fragmented RNA or DNA and the like. In certain embodiments, the nucleic acid sample comprises at least one of messenger RNA, intronic RNA, exonic DNA, and non-coding RNA. The nucleic acid may be prepared for hybridization according to any manner as would be understood by those having ordinary skill in the art. It should also be appreciated that the sample may be an isolated nucleic acid sample, or it may form part of a lysed cell, or it may be an intact living cell. Samples may further be individual cells, or a population of cells, such as a population of cells corresponding to a particular tissue. It should be appreciated that there is no limitation to the size or type of sample, provided the sample includes at least one nucleic acid therein. For example, the sample may be derived or obtained from one or more eukaryotic cells, prokaryotic cells, bacteria, virus, exosome, liposome, and the like. In certain embodiments, a sample is fixed. For example, in one embodiment, a living cell or tissue is provided and fixed prior to application of one or more probes. As described herein, in one embodiment, the sample is fixed using a non-crosslinking fixative, which allows for shorter hybridization times. In certain embodiments, a non-crosslinking fixative allows for the use of a higher probe concentration which shortens hybridization times. In certain embodiments, the non-crosslinking fixative is an alcohol-based fixative

### Method

Accordingly, the present invention relates to a method for reliably detecting one or more target nucleic acids in a sample using RNA FISH in a very short period of time. The method can be generally described as including the following steps.

In one embodiment, the method comprises providing a sample. As discussed elsewhere herein, the sample may be derived or obtained from one or more eukaryotic cells, prokaryotic cells, bacteria, virus, exosome, liposome, and the like. In certain embodiments, the sample is obtained from a subject (e.g., a biological sample), including, for example, from a human, swine, or avian subject. In one embodiment, the sample is a cell. In another embodiment, the sample is a tissue sample. In one embodiment, the sample is a body sample, including, for example, blood, urine, skin, fat, saliva, and the like.

In one embodiment, the method comprises fixing the sample. It is discovered herein that the use of a non-crosslinking fixative allows for significantly shorter hybridization times with similar results to current overnight hybridization protocols. The present invention includes the use of any compositions and methods for non-crosslinking fixation known in the art. In one embodiment, the method comprises fixing the sample using an alcohol-based fixative. For example, in one embodiment, the method comprises fixing the sample using a fixative comprising methanol. In another embodiment, the method comprises fixing the sample using a fixative comprising ethanol. For example, in one embodiment, the method comprises administering an alcohol-based fixative to the sample, thereby fixing the sample. Fixation of the sample using the non-crosslinking fixatives may be done under any suitable conditions which results in the fixation of the sample. For example, in one embodiment, the sample is contacted with the non-crosslinking fixative for about 1 second to about 5 hours. In one embodiment, the sample is contacted with the non-crosslinking fixative (e.g., methanol) for about 2 minutes. In another embodiment, the sample is contacted with the non-crosslinking fixative for about 10 minutes. In one embodiment, the sample is contacted with the non-crosslinking fixative at a temperature of about −80° C. to about 50° C. In one embodiment, the sample is contacted with the non-crosslinking fixative at a temperature of about −20° C. In another embodiment, the sample is contacted with the non-crosslinking fixative at room temperature (e.g., about 20-23° C.). In one embodiment, following incubation with the non-crosslinking fixative, the sample is washed.

In one embodiment, the method comprises contacting the fixed sample with one or more probes. In certain embodiments, the non-crosslinking fixative allows for the use of a higher concentration of probes thereby shortening the hybridization time. For example, in one embodiment, the fixed sample is contacted with a hybridization solution comprising a probe concentration of about 0.01 mM to about 1000 mM. In another embodiment, the fixed sample is contacted with a hybridization solution comprising a probe concentration of about 0.1 mM to about 50 mM. In yet another embodiment, the fixed sample is contacted with a hybridization solution comprising a probe concentration of about 0.1 mM to about 20 mM. In a further embodiment, the fixed sample is contacted with a hybridization solution comprising a probe concentration of about 0.22 mM to 14.2 mM. In another embodiment, the fixed sample is contacted with a hybridization solution comprising a probe concentration of about 3 mM to 4 mM. In a further specific embodiment, the fixed sample is contacted with a hybridization solution comprising a probe concentration of about 3.56 mM. The hybridization solution may further comprise any additional suitable components known in the art. For example, in one embodiment, the hybridization solution comprises formamide, saline-sodium citrate, and dextran sulfate. As discussed elsewhere herein, the present invention allows for shorter hybridization times. While previous methods typically require overnight incubation with the probes during hybridization, the present method allows for hybridization times on the order of seconds or minutes. For example, in one embodiment, the method comprises contacting the sample with the hybridization solution for less than about 5 hours. In one embodiment, the method comprises contacting the sample with the hybridization solution for less than about 2 hours. In one embodiment, the method comprises contacting the sample with the hybridization solution for less than about 1 hour. In one embodiment, the method comprises contacting the sample with the hybridization solution for less than about 10 minutes. In one embodiment, the method comprises contacting the sample with the hybridization solution for less than about 5 minutes. In one embodiment, the method comprises contacting the sample with the hybridization solution for less than about 1 minute. Thus, in the embodiments disclosed herein, the method comprises contacting the sample with the hybridization solution for less than about 5 hours to less than about 1 minute and any and all ranges therebetween.

In certain embodiments, the shorter hybridization time used in the method reduces the amount of drying, thereby allowing for the use of a small quantity of hybridization solution. For example, in one embodiment, the method allows for the use of about 10 fold less hybridization solution compared to standard methods. This allows for a greater concentration of probe to be used without an appreciable change in the overall amount of probe. For example, in one embodiment, the method comprises contacting the sample with about 0.1-10 μL of hybridization solution. In another embodiment, the method comprises contacting the sample with about 5 μL of hybridization solution.

Hybridization of the probes to the sample may be performed in any suitable hybridization conditions known in the art. For example, in one embodiment, the sample is contacted with the hybridization solution at a temperature of about 0° C. to about 100° C. In one embodiment the sample is contacted with the hybridization solution at a temperature of about 37° C. In one embodiment, following incubation with the hybridization solution, the sample is washed. In certain embodiments, the use of non-crosslinking fixatives allows for shorter wash times. For example, in one embodiment, the washing of the sample comprises three separate one minute incubations with a wash solution. The wash solution may be any standard or suitable buffer or solution known in the art.

In certain embodiments, the sample is imaged and analyzed for the presence, location, or amount of one or more targets. Imaging of the sample may be done using any suitable imaging instrumentation and software systems known in art.

In certain embodiments, the Turbo FISH method described herein allows for the detection of one or more targets in a sample in a total time of about 5 minutes to about 4 hours. This allows for FISH to be utilized in rapid diagnostic applications, which otherwise would be impossible or impractical.

### Test Device

Accordingly, the present invention provides a fluidics device for use with the detection methods of the invention. The fluidics device comprises a liquid reservoir with two openings. One opening may be used as an input for introducing a fluid, including a sample (e.g., a biological sample) comprising or suspended in a fluid and/or a test reagent or buffer, into a liquid reservoir, and the other opening may be used as an outlet for fluids exiting the liquid reservoir. The openings may be configured as channels. The openings may be connected to one or more additional devices for automated sample processing. The fluidics device may be made of optically transparent material (e.g., optically transparent glass or plastic). In particular, the transparent components of the device permit the contents of the liquid reservoir to be viewed or imaged (e.g., by a microscope). An object of interest is any material entity to be stimulated, studied, investigated or otherwise influenced by means of the fluidic device. In certain embodiments, the sample to be analyzed by the test device comprises a cell. The liquid reservoir may include a means for holding a cell in place (e.g., a filter, such as a micropore filter). In a specific embodiment, the device holds a plurality of cells in substantially the same plane or focal plane, such as to facilitate imaging of the cells. The fluidic device can be operated to deliver solution to the open volume in order to superfuse an object of interest (e.g., a cell). The fluidic device can be operated to extract solution from the open volume in order to collect a release from an object of interest (e.g., a cell).

The fluidic device can have a flat rectangular shape. The fluidic device can be between about 1 mm and 10 cm wide. The fluidic device can be between about 0.1 and 5 mm high. The fluidic device can be between about 1 mm and 10 cm long. The volume of the liquid reservoir of the fluidic device can be between about 10 μL and 10 mL. The liquid reservoir of the fluidic device can be between about 1 mm and 10 cm wide. The liquid reservoir of the fluidic device can be between about 0.1 mm and 1 cm high. The liquid reservoir of the fluidic device can be between about 1 mm and 10 cm long.

Multiple RNA FISH assays can be integrated onto a single device (e.g., a chip), enabling the multiplexed detection of several RNA FISH markers. This integration can allow multiple virus-types to be to be rapidly screened in a practical clinical setting.

In particular embodiments, the fluidics device is a microfluidics device. The microfluidic device can have a flat rectangular shape. The microfluidic device can be between about 1 mm and 10 cm wide. The microfluidic device can be between 0.1 and 5 mm high. The microfluidic device can be between about 1 min and 10 cm long.

The microfluidics device may comprise one or more wells which provide a liquid reservoir. The microfluidic device can have between 1 and 1000 wells or more. In various embodiments, the microfluidic device can have 2, 4, 8, 16, 32, 64, 128, 256, 512, 1024 wells or more. Each device can include N=2ⁿ isolated detection regions with a theoretical upper limit set only by the number of cells obtained from the patient. Such a device enables panels of assays to be run simultaneously, each detecting specific variants or even different viruses in the same device.

The wells in the microfluidic chip can be arranged in one row. The wells in the microfluidic chip can be arranged in a staggered pattern. The wells in the microfluidic chip can be arranged in two rows or more. The wells may be arranged in an array.

The microfluidic device can have circular wells. The microfluidic device can have rectangular wells. The microfluidic device can have rectangular wells with rounded corners.

The wells of the microfluidic device can be of equal size. The wells of the microfluidic device can be of different sizes. The wells of the microfluidic device can have a volume between about 10 pL and about 50 μL. The wells of the microfluidic device can have a volume between about 50 μL and about 100 μL. The wells of the microfluidic device can have a volume between about 100 pL and about 500 μL.

A well-to-well separation distance in the microfluidic device can be between about 4 mm and about 12 mm. The well-to-well separation distance can be about 6 mm. The well-to-well separation distance can be about 9 mm. The well-to-well separation distance can be about 4.5 mm.

One or more microfluidic channels can be in direct fluid communication with one or more wells. A channel can be connected with a well through an orifice that is smaller in diameter than a bottom diameter of the well. Pneumatic connectivity can be used to supply pressure to the one or more wells. The wells can be in communication with a common pressure source. Each well can be in individual communication with a pressure source. One or more tubes adapted and configured to facilitate pneumatic connectivity with the one or more wells can be used to control the pressure in the wells. The tubes can have an inner diameter between about 0.5 mm and about 1 mm. The tubes can have an inner diameter between about 0.5 mm and about 2 mm.

The liquid reservoir or wells of the device may comprise one or more RNA FISH probes or reagents. The RNA FISH probes or reagents may be pre-loaded at any point prior to performing the RNA FISH assay for ease of use. The ease of use can be validated by handing over devices to medical personnel and quantifying user variability.

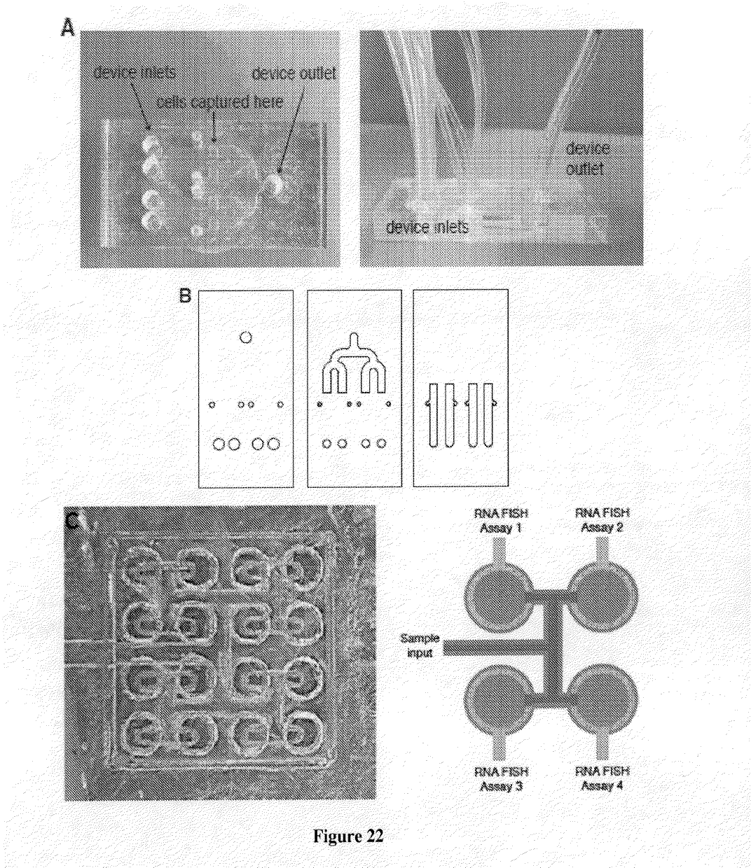

In various embodiments, the device has multiple wells. In particular embodiments, the cells from a clinical sample are evenly split into isolated regions for micropore based capture and hybridization (e.g., into 4 regions, see FIGS. 22A and 22B). In a specific embodiments, a microfluidic geometry is used in which cells from the clinical sample are evenly split into sixteen isolated regions for micropore based capture and hybridization. To evenly distribute the sample, a “shower head” geometry may be implemented in which symmetric branching is used to split the flow evenly to sixteen 1 mm² holes above the micropore filter (FIG. 22C). Cells trapped in each of the individual trapping regions are exposed to a unique RNA FISH probe. Continuous negative pressure from the output ensures that no mixing occurs between the detection regions. By splitting the sample flow into multiple wells, robust flow can be maintained.

In additional embodiments, metered quantities of RNA FISH reagents are preloaded into tubes separated by air bubble spacers while loading the biological sample (e.g., nasal swab/aspirate) is loaded into a separate reservoir. The sample is loaded and concentrated, and the RNA FISH reagents are drawn through using a single syringe held at negative pressure. To remove the air bubble spacers, a micro-scale bubble trap is used (Jong Hwan and Shuler, Biomedical microdevices, 2009; 731-738), preventing the bubbles from interfering with imaging or from releasing the cells from the micropore filter. This design strategy requires no moving parts, electricity, or external instrumentation.

Another aspect of the invention provides a method for utilizing a fluidic or microfluidic device. The method includes: providing a device as described herein; and/or operating the microfluidic device (e.g., by introducing a fluid or liquid sample into a reservoir of the fluidic or microfluidic device). In a particular embodiment, the fluidic or microfluidic device is used to detect one or more target nucleic acids in a sample using RNA FISH in a very short period of time, in accordance with the aforementioned detection methods.

The method may further include positioning the device adjacent to a microscope (e.g., near the objective lens of the microscope to allow the contents of the device to be observed). This aspect of the invention can have a variety of embodiments. The microscope can be an upright microscope. The microscope can be an inverted microscope. The microscope comprises an objective lens having magnification of at least about 4×, 10×, 20×, 30×, 60×, 100× or more. The method can further include utilizing a means to position the fluidic or microfluidic device (e.g. micromanipulator, platform, arm, and the like).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### A Method for Rapidly Performing Ribonucleic Acid Fluorescence In Situ Hybridization

Advances in RNA fluorescence in situ hybridization (RNA FISH) have allowed practitioners to detect individual RNA molecules in single cells via fluorescence microscopy, enabling highly accurate and sensitive quantification of gene expression. However, current methods typically employ hybridization times on the order of 2-16 hours, limiting its potential in applications like rapid diagnostics. A set of conditions for RNA FISH (dubbed Turbo RNA FISH) is presented here that allows for accurate measurements with no more than 5 minutes of hybridization time and 3 minutes of washing, and with hybridization times as low as 30 seconds while still producing quantifiable images. The method is simple and cost effective, and has the potential to dramatically increase the throughput and realm of applicability of RNA FISH. Both the fixation conditions and hybridization conditions have been optimized to achieve these results, showing there is a tradeoff between hybridization speed and probe concentration.

The materials and methods employed in these experiments are now described.

### Cell Culture

A549 cells (ATCC CCL-185), HeLa cells, and primary human foreskin fibroblasts (ATCC CRL-2097) were cultured in Dulbecco's modified Eagle's medium with Glutamax (DMEM, Invitrogen) supplemented with 10% fetal bovine serum and penicillin/streptomyocin. WM983b cells were cultured in melanoma isolation media containing 80% MCDB153, 18% Leibovitz's L-15, 2% fetal bovine serum, 1.68 mM CaCl₂, and penicillin/streptomyocin.

### Formaldehyde Fixation

Cells were fixed for 10 minutes in 4% formaldehyde/10% formalin in IX phosphate buffered saline solution at room temperature. Following fixation, cells were washed twice with 1× phosphate buffer solution and then permeabilized with 70% ethanol and stored at 4° C. for at least overnight.

### Alcohol Fixation

Cells were fixed in pre-chilled ethanol or methanol (−20° C.) for 2-10 minutes. Following fixation, RNA FISH or Turbo RNA FISH was performed immediately.

### RNA FISH

To perform RNA FISH, the protocol in Raj et al. was followed (Raj et al., 2008, Nat Methods 5:877-879) with minor modifications. Cells were pre-washed with wash buffer containing 10% formamide and 2× saline sodium citrate (SSC). Hybridization was then performed by adding the appropriate amount of probe to a hybridization buffer consisting of 10% formamide, 2×SSC, and 10% dextran sulfate (w/v). The final volume for hybridization was 50 μL, and the final probe concentrations ranged from 0.22 mM to about 14.23 mM for TOP2A and 0.31 mM to about 19.6 mM for TBCB. The samples were hybridized overnight in a humidified chamber at 37° C. Following hybridization, the samples were washed twice with wash buffer for 30 minutes at 37° C. The samples were then imaged in 2×SSC.

### Turbo RNA FISH

For Turbo RNA FISH, the alcohol was removed from previously fixed samples and hybridization was performed with 5 μL of hybridization buffer containing the specified amount of probe, 10% formamide, 2×SSC, and 10% dextran sulfate (w/v). The samples were hybridized for the specified time on a covered hot plate at 37° C. Following hybridization, the samples were washed three times for one minute at 37° C. with pre-warmed wash buffer. The samples were then imaged in 2×SSC.

Turbo iceFISH

For Turbo iceFISH, a previously described protocol was followed, (Levesque et al., 2013, Nature Methods, doi: 10.1038/nmeth.2372) but with methanol fixed cells, a 10-fold higher probe concentration and a 5 minute hybridization time. Probes which “paint” chromosome 19 were used (Levesque et al., 2013, Nature Methods, doi: 10.1038/nmeth.2372), with the chromosome paint labeled with Alexa 594. iceFISH was performed in HeLa cells, which have two normal copies of chromosome 19 and two derivative chromosomes, t(13;19) and t(6;19).

### Turbo SNP FISH

For Turbo SNP FISH, a previously described protocol was followed (Levesque et al., 2013, Nature Methods, in press), but with methanol fixed cells, higher probe concentration and shorter hybridization times. A 5 minute hybridization in 5 μL containing 30 mM of each single DNA oligonucleotide, 100 mM of the DNA mask, and a 20-fold increased concentration of guide probe as compared to conventional RNA FISH conditions, were used. Notably, a 20 minute post-fix in formaldehyde after the hybridization was performed to prevent probes from dissociating. Further, glucose oxidase was used to prevent photobleaching of the Cy5 dye (Raj et al., 2008, Nat Methods 5:877-879). Probes which detect the BRAF V600E mutation were used (Levesque et al., 2013, Nature Methods, in press), as well as one that targets a portion of BRAF that is the same on both alleles as a control. Image analysis was the same as that described previously (Levesque et al., 2013, Nature Methods, in press). All the experiments were performed in WM983b cells, which are heterozygous for the V600E mutation.

### Image Acquisition

All samples were imaged on a Nikon Ti-E inverted fluorescence microscope using a 100× Plan-Apo objective (numerical aperture of 1.43) and a cooled CCD camera (Andor iKon 934). Three-dimensional stacks of fluorescence images were sequentially acquired in four different fluorescence channels using filter sets for DAPI, Cy3, Alexa 594, and Atto 647N. The exposure times ranged from 2-3 seconds for most of the dyes except for DAPI, for which ˜50 ms exposures were used. The spacing between consecutive planes in the stacks was 0.3 μm. The filter sets used were 31000v2 (Chroma), 41028 (Chroma), SP102v1 (Chroma), a custom set from Omega as described in 13, SP104v2 (Chroma) and SP105 (Chroma) for DAPI, Atto 488, Cy3, Alexa 594, Atto 647N and Atto 700, respectively.

### Image Analysis and Quantification

After imaging, the data was put through an image analysis pipeline for semi-automated spot recognition (Levesque & Raj, 2013, Nat Methods, doi:10.1038/nmeth.2372). The analysis pipeline was implemented in MATLAB. Briefly, the method for analysis involved running the images through a linear filter designed to enhance spots around the size of those observed, then finding all regional maxima within the filtered image, and then counting the number of regional maxima below a variety of thresholds (Raj et al., 2008, Nat Methods 5:877-879). A threshold where the number of regional maxima changes the least upon changing the threshold (i.e., the number of spots is least sensitive to moving the threshold) was then determined manually. To quantify sensitivity of the threshold, the derivative of the logarithm of the graph of the number of regional maxima below varying thresholds was taken. The derivative was smoothed before quantifying to avoid noise due to local variations in the graph.

The results of the experiments are now described.

### RNA FISH Enables Single Molecule Detection

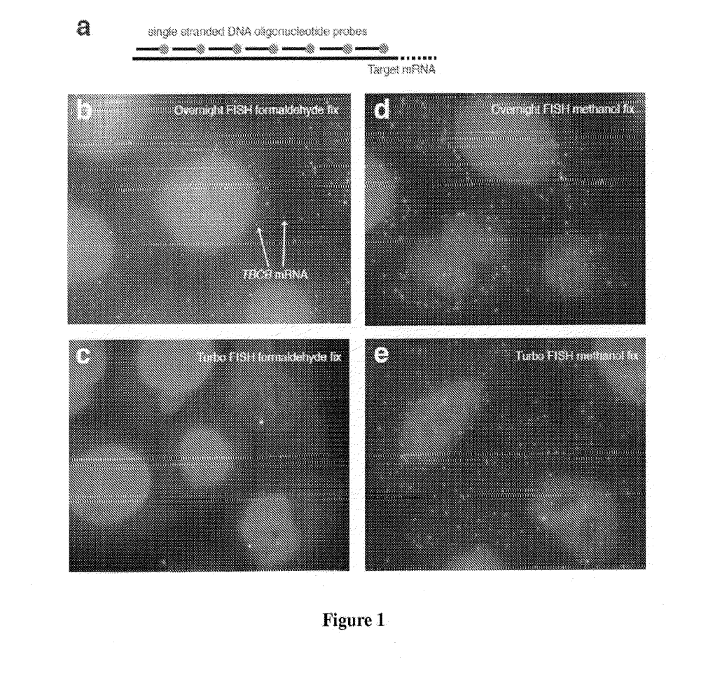

The method employed for RNA FISH involved the use of several 20-base long single-stranded DNA oligonucleotides, each individually labeled (Raj et al., 2008, Nat Methods 5:877-879; Raj & Tyagi, 2010 Meth Enzymol 472:365-386). These oligonucleotides were designed to bind to different segments of the target RNA via Watson-Crick base pairing, and the combined fluorescence from all the fluorophores at the single RNA led to a fluorescent spot of intensity much higher than that of the background. A representative image for a probe targeting the gene TBCB is shown in FIG. 1B,

### Fixation Conditions

Traditionally, the hybridizations were performed overnight in order to obtain strong signals. In order to perform rapid RNA FISH, it was initially reasoned that the hybridization kinetics could be improved by increasing the concentration of probe included in the hybridization. Thus, it was initially attempted to speed hybridization by simply increasing the amount of probe in the hybridization solution. It was found, however, that despite increasing the concentration 20 fold, the signals were greatly diminished at hybridization times of 5 minutes (FIGS. 1B, 1C). The normal protocol utilizes cells that are fixed with formaldehyde, and it was considered whether the cross-links created by this form of fixation could impede the ability of the oligonucleotide probes to find their targets.

To investigate this possibility, hybridization was performed with both ethanol- and methanol-fixed cells (each fixation performed with −20° C. cold ethanol/methanol), both of which do not generate cross-links. It was found that both alcohol-based fixatives performed considerably better (FIGS. 1D, 1E), generating images that were equivalent to those obtained by overnight hybridization with standard conditions. Also of note is that the washing time was reduced in these cases to three one-minute washes, for a total of 3 minutes. Note that fixation using methanol or ethanol at room temperature also produces similarly equivalent RNA FISH signals.

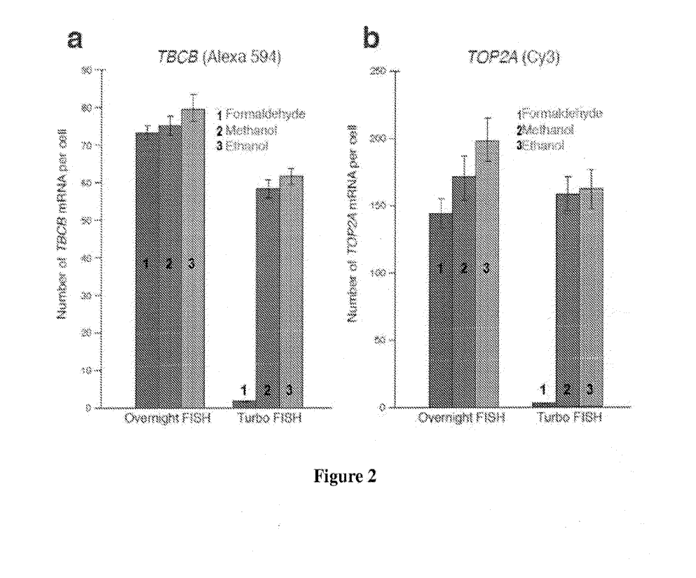

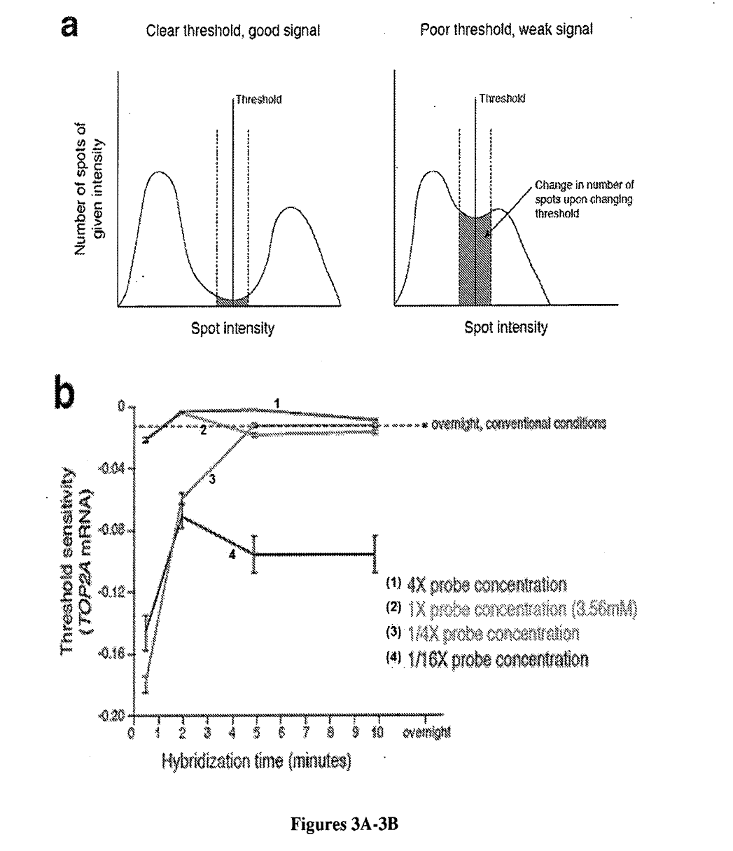

The number of mRNA detected in all conditions was then quantified using software similar in principle to that applied previously (Raj et al., 2008, Nat Methods 5:877-879). It was found that after performing overnight hybridization, the same number of RNA per cell was obtained with all fixation methods, but for rapid hybridizations performed at high concentrations, both alcohol-based fixatives gave similar results to those obtained from the overnight hybridizations, whereas the formaldehyde fixed cells performed much more poorly (FIGS. 2A, 2B). Methanol-fixed cells were used for the remainder of the rapid hybridization experiments. These results establish that in this invention the hybridization times were decreased by an order of magnitude as compared to prior art protocols.

### Relationship Between Concentration and Hybridization Time

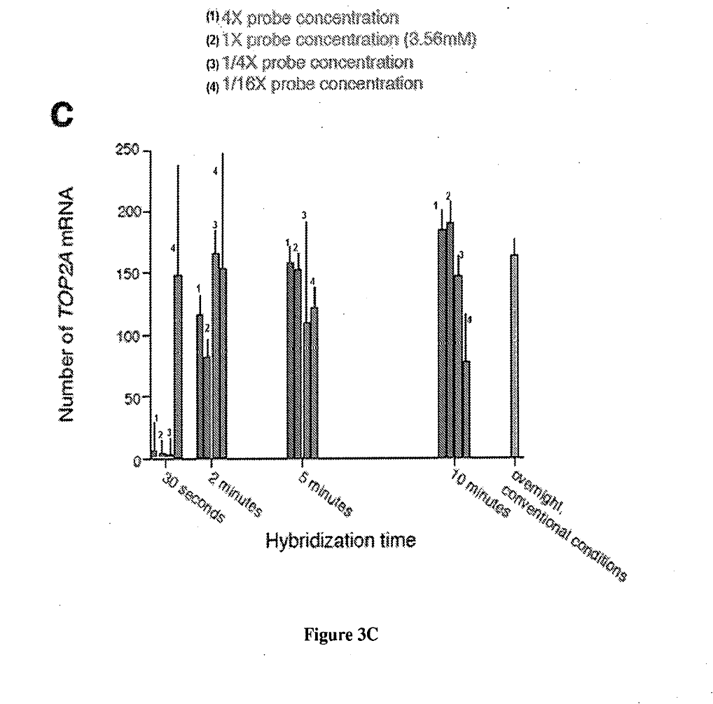

The degree to which there is a tradeoff between increasing the concentration of the probe and the hybridization time for rapid hybridization in methanol-fixed cells was then explored. In order to do so, a means to assess and compare the quality of the signal in these various conditions was needed. Ultimately, a metric based on the sensitivity of the threshold between signal and background was used (FIG. 3A). Briefly, a linear filter designed to enhance spot-like signals was first used. All candidate spots were then found by locating all regional maxima. These candidate spots consist of two populations, one corresponding to background spots and one corresponding to the target RNA molecules. When the signals are clear and quantifiable, the intensities of the RNA spots should be nicely separated from those of the background spots (FIG. 3A). However, if the RNA spots are not of high quality, then the spot intensities of the two populations can blend together, making it difficult to accurately quantify the number of true RNA spots within the image (FIG. 3A). To quantify this difference, the degree of separation in the intensities of the two subpopulations was measured by essentially measuring the sensitivity of the threshold separating the two; i.e., once the threshold was set, if the threshold was moved slightly higher or lower, the relative change in the number of RNA detected was measured (FIG. 3A). It was found that this metric for quantification captured the qualitative visual differences between conditions. Further noted is that metrics such as spot intensity and average spot count can be somewhat misleading (FIG. 3C). In the former case, it has been found that spot intensity need not be particularly great in order for accurate quantification. In the latter, it has been found that manual thresholding can often yield comparable counts, but the threshold itself is so ill-defined that a different person might very well come up with completely different results; thus, the focus was primarily on the sensitivity metric to quantify signal quality.

Data from A549 cells, a common cancer cell type that has been found overall to be more difficult to perform rapid hybridizations in (hence providing a stringent test of the method) is presented herein. RNA FISH was performed (targeting TOP2A mRNA) over a range of hybridization times from 30 seconds to 10 minutes and probe concentrations ranging from the conventional probe concentration to 80 fold greater (approximately 0.22 mM to 14.2 mM). Throughout, results were also compared to the traditional overnight hybridization protocol (FIGS. 3B, 3C). It was found that readily quantifiable signals were obtained after 5 minutes of hybridization in the A549 cells (FIGS. 3B, 3C). It was found that there is a clear tradeoff, in that higher concentrations of probe in the hybridization solution allows for shorter hybridization times. The exact amount of time and concentration that should be used in these cases will of course depend on the constraints of the problem at hand, but it is believed that a 5 minute hybridization at a probe concentration of 3.56 mM would be practical in many scenarios. In some cases, it is possible to perform rapid hybridizations in as little as 30 seconds with high concentrations of probe.

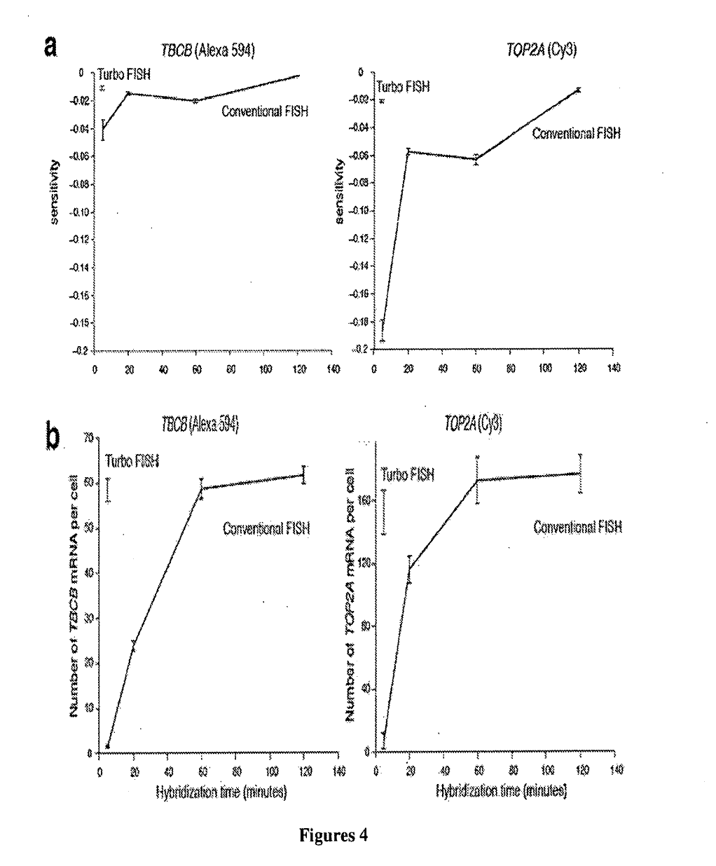

For comparison, the same analysis was performed by using the concentrations and wash protocol used for conventional overnight RNA FISH, except performing the hybridization for various amounts of time. It was found that poorly quantifiable signals were obtained, as indicated by the sensitivity metric, once the hybridization time went below 2 hours, which is 24-fold as much time as the rapid hybridization assay (FIGS. 4A, 4B).

iceFISH and SNP FISH

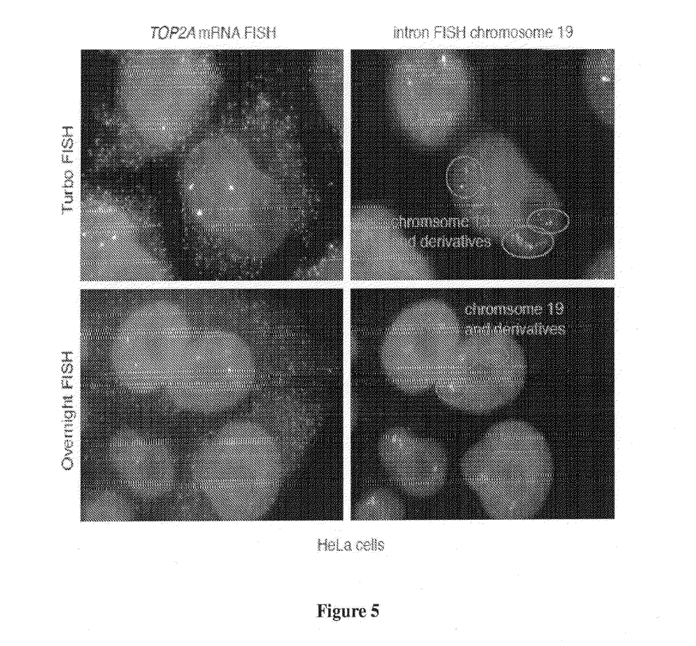

Two variants of single molecule RNA FISH have recently been developed: 1. a method based on targeting introns that reveals chromosome structure and transcriptional activity (intron chromosomal expression FISH or iceFISH; Levesque et al., 2013, Nature Methods, doi: 10.1038/nmeth.2372), and 2. a method that utilizes both a new probe design and spot colocalization analysis to enable the detection of single nucleotide differences on individual transcripts (SNP FISH; Levesque et al., Nature Methods, in press). It was examined whether these methods would work in the rapid hybridization format. For iceFISH, an intron-based chromosomal “paint” was constructed that targets chromosome 19. It was found that the iceFISH signals were comparable to those obtained via conventional overnight FISH using the described rapid hybridization conditions (FIG. 5).

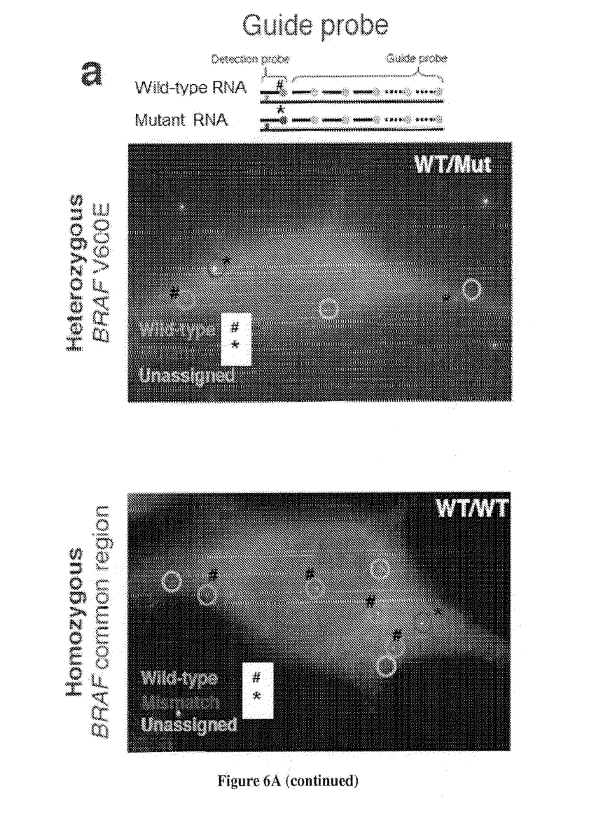

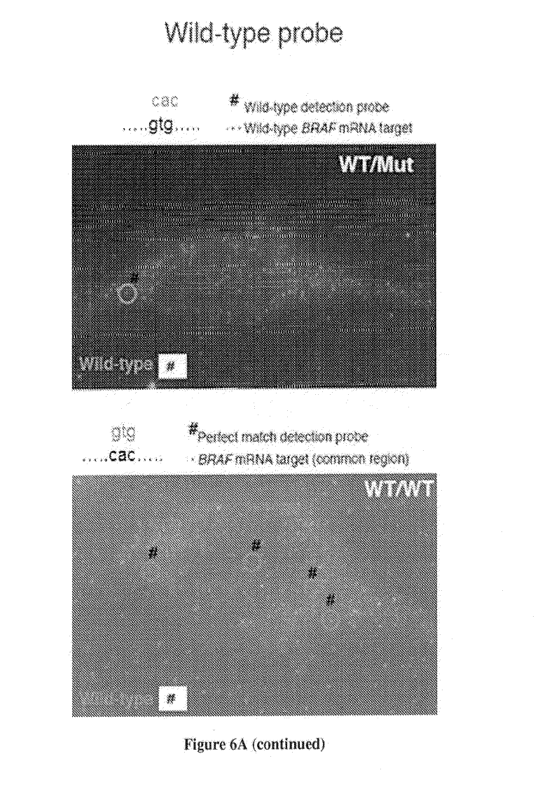

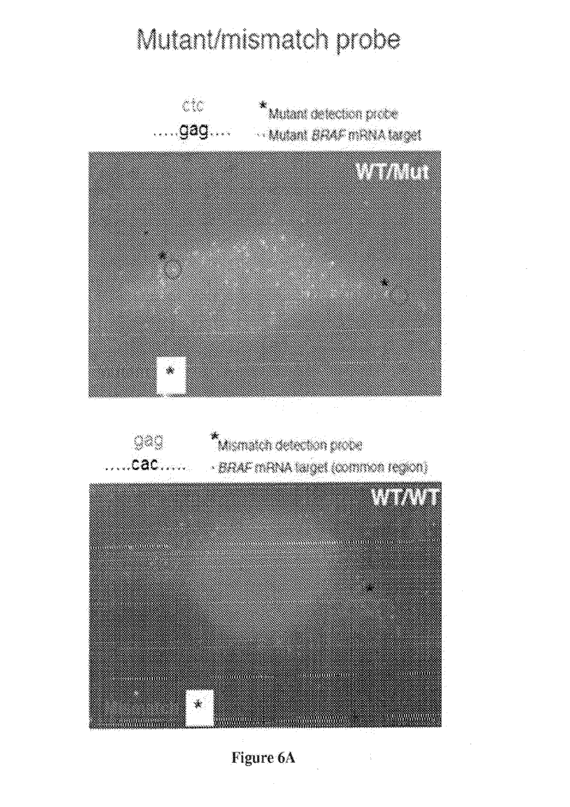

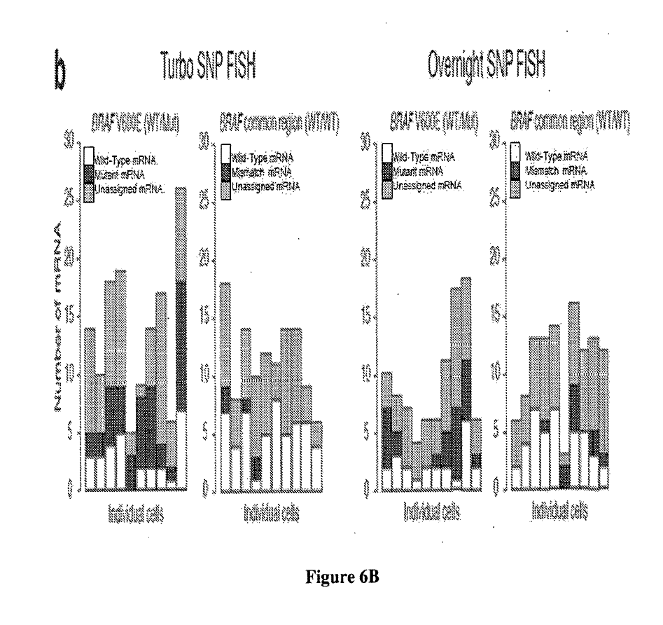

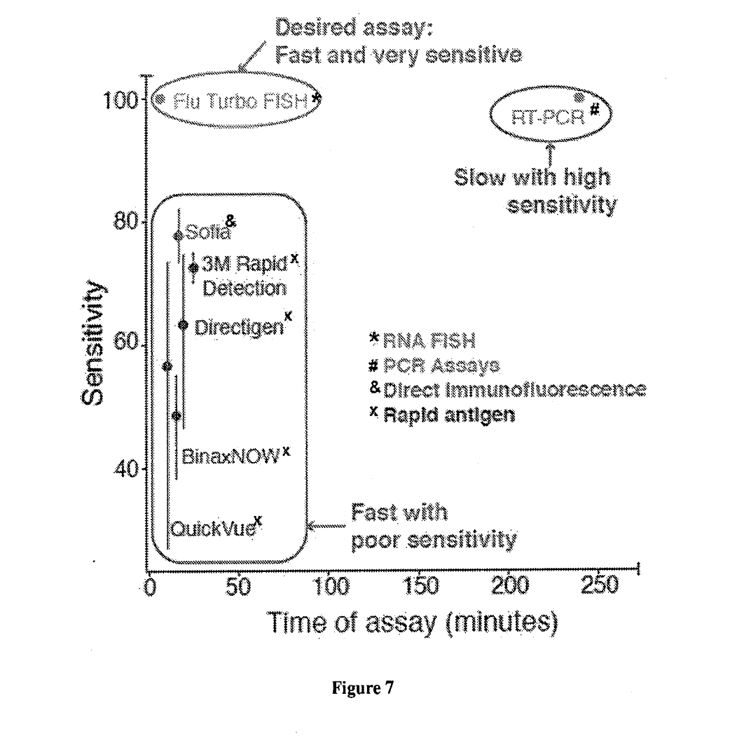

For SNP FISH, an approach was used utilizing a single oligonucleotide “SNP detection” probe hybridized to a “mask” oligonucleotide that leaves just a short “toehold” region available to nucleate binding to the target RNA (FIG. 6). The toehold region is short enough (5-10 bases) that it provides discrimination of single-base mismatches, but upon the binding of the correct probe, the mask dissociates via strand displacement (Zhang et al., 2009, J Am Chem Soc, 131, 17303-17314), leading to the formation of a long (˜20-30 base) hybrid that provides stability. Meanwhile, the rest of the target RNA was labeled using conventional RNA FISH probes (which are called “guide probes”), which inform where the target RNA are within the cell. Using colocalization between the SNP detection probe and the guide probe, each RNA could be assigned based on whether or not it has the SNP. Previous work demonstrated that this approach works under conventional RNA FISH conditions (Levesque et al., Nature Methods, in press). To check whether SNP FISH was able to be performed in rapid hybridization conditions, a higher concentration of probes and a shortened hybridization times (5 minutes) was used in in methanol fixed cells. Turbo SNP FISH was tested in WM983b cells, which are heterozygous for the V600E mutation in the BRAF gene. Probes targeting the V600E BRAF mutation or a region common to both alleles on the BRAF mRNA as a control for non-specific binding were utilized (FIG. 6A). It was found that in both Turbo SNP FISH and conventional overnight SNP FISH, the probes targeting the heterozygous base in BRAF indeed showed roughly equivalent levels of both mutant and wild-type mRNA (FIG. 6A, top). The probes targeting the region common between the two alleles identified virtually all the mRNA as being wild-type in both turbo and conventional conditions, showing that the rate of cross-hybridization remained low even with rapid hybridization conditions (FIG. 6A). Quantitatively, the results from both turbo and conventional SNP FISH were similar, with the turbo SNP FISH detection efficiency of 50.4% being similar to that obtained from overnight hybridization (45.2%) (FIG. 6B).

### Ultra-Rapid RNA FISH for Detection of Influenza Virus in Clinical Samples

The RNA FISH methodology was used for the rapid detection of influenza virus. Cell culture systems were used to establish the parameters of the assay and this data was applied to assays of clinical samples.

The genome of the influenza virus consists of single stranded RNA, making it an ideal target for RNA FISH using single stranded DNA oligonucleotide probes. A set of probes suitable for a clinical diagnostic, provides one or more rapid, specific and quantifiable signals. Clinical samples may also contain other RNA viruses such as respiratory syncytial virus that should be excluded from detection. The influenza virus engenders a variety of potential probe targets. There is the viral genome itself (vRNA), but also the viral mRNA that are transcribed from those vRNA. Data seems to indicate that the vRNA is at lower abundance than the viral mRNAs (Chou et al., Proc Natl Acad Sci USA. 2012; 109(23): 9101-6; Chou et al., PLoS Pathog. 2013; 9(5):e1003358). Also, a full diagnostic includes image analysis algorithms that can robustly detect infection and separate it from background.

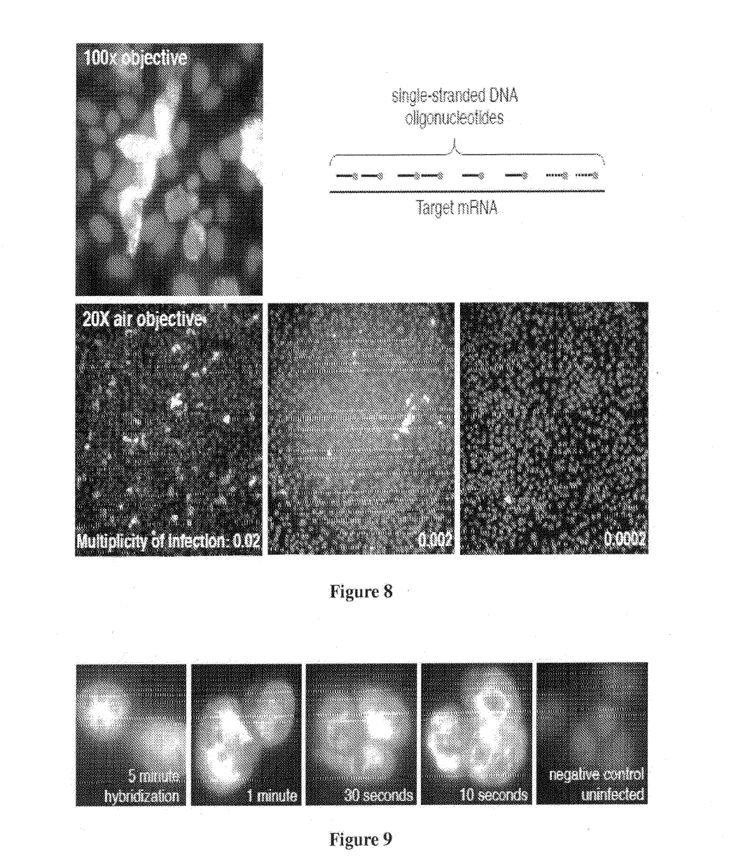

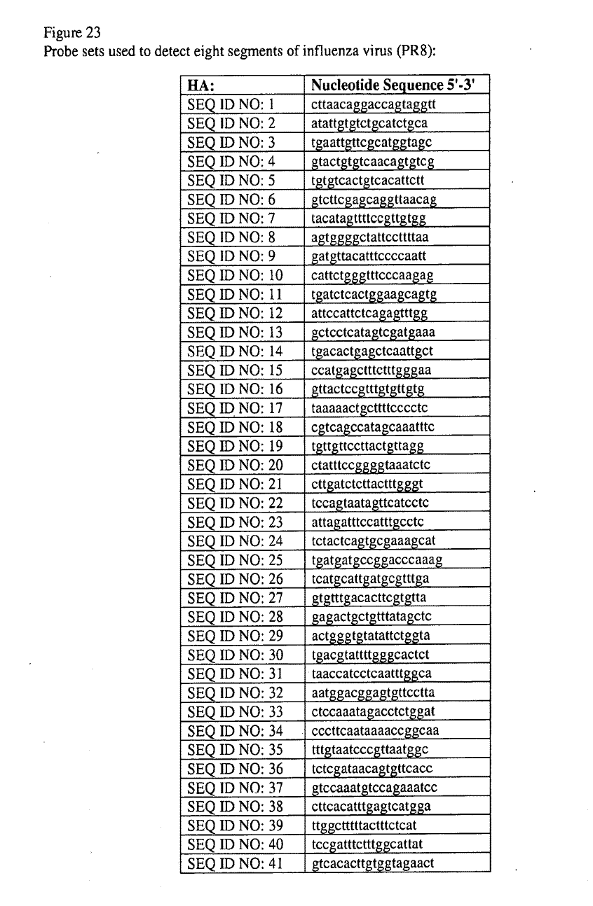

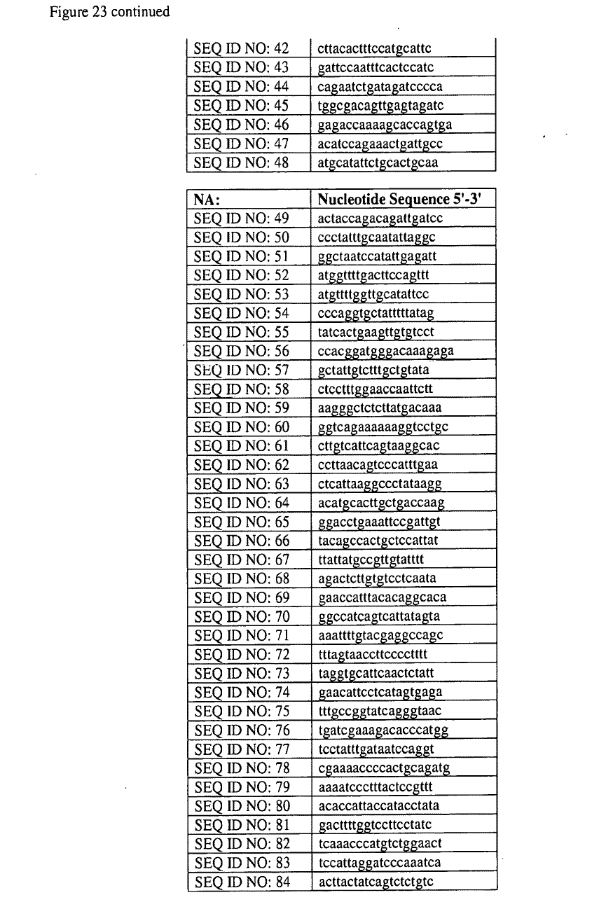

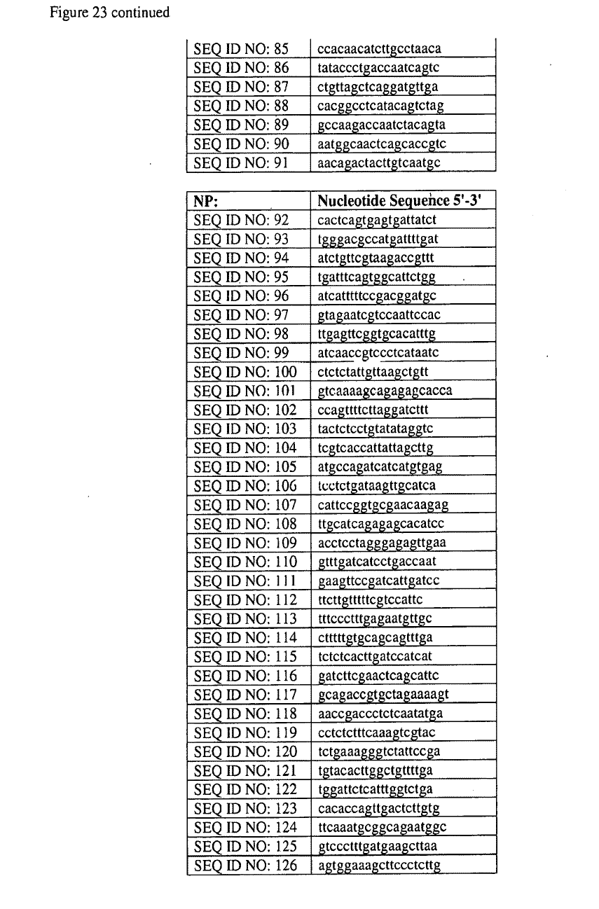

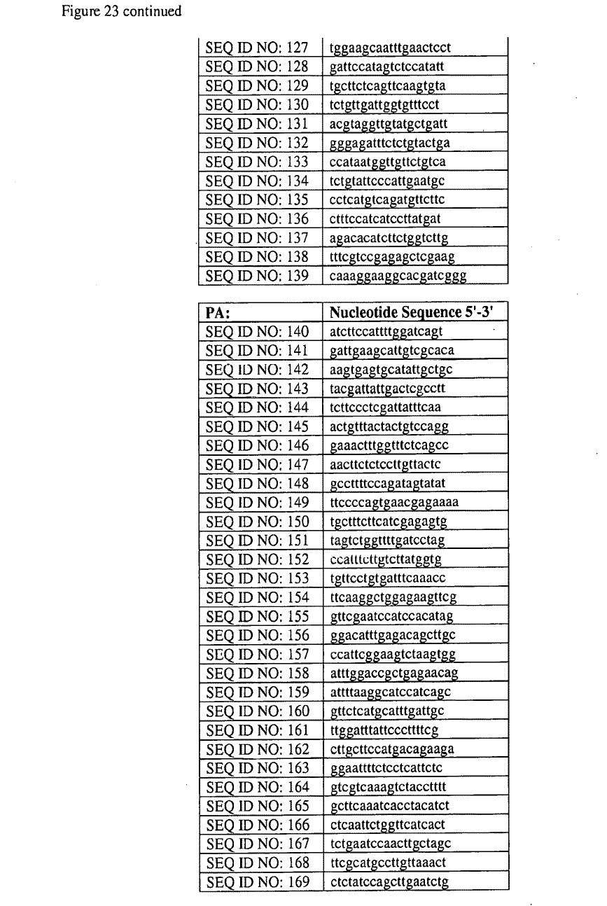

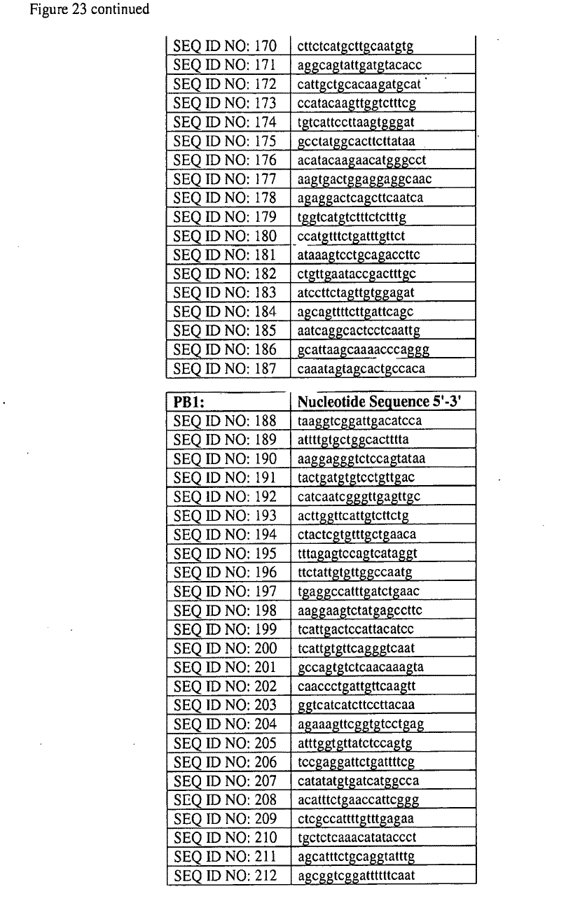

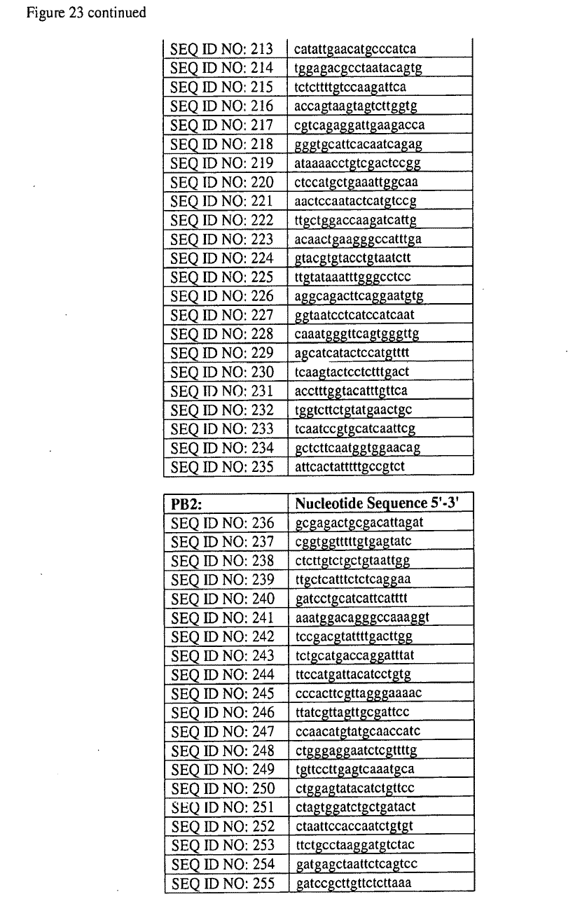

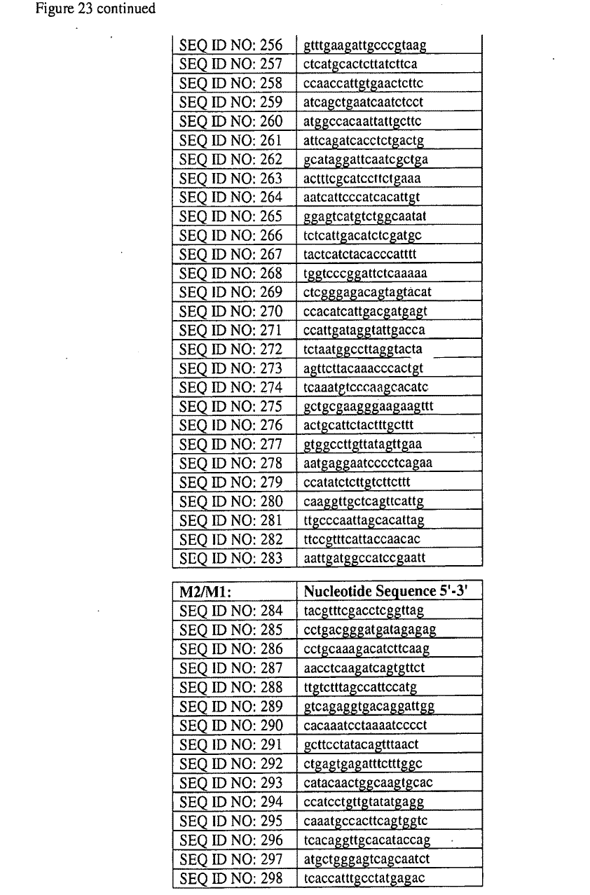

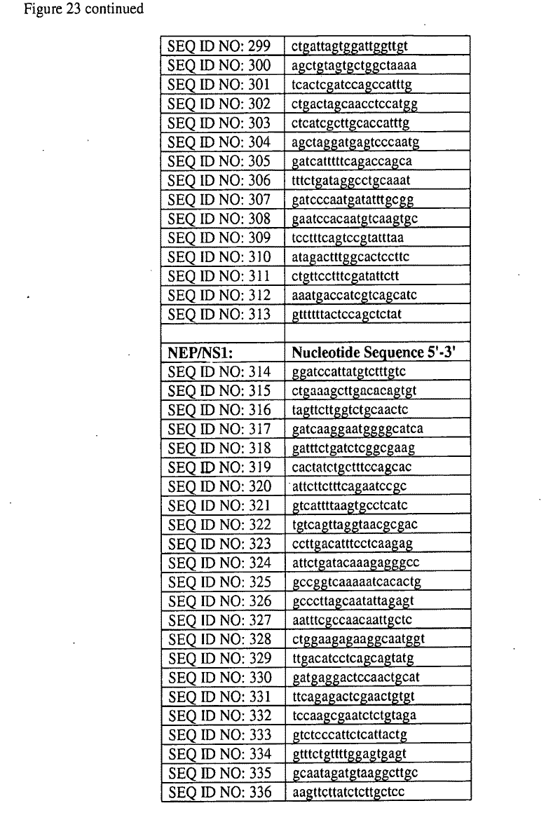

To establish the feasibility of ultra-rapid detection of influenza by RNA FISH, experiments were performed in the widely used Madine-Darby canine kidney (MDCK) cell line (Meguro et al., J. Clin. Microbiol. 1979; 9(2): 175-9), which supports influenza virus. This system enabled rapid testing of different probes to determine which gave reliable signals. Initially, a set of 336 single-stranded DNA oligonucleotide probes for RNA FISH were designed that collectively targeted all 8 segments of the influenza viral mRNA. The probes brightly illuminated cells infected with influenza virus, with little to no signal in uninfected cells (FIG. 8). The probes and oligonucleotides used in the experiment are depicted at FIG. 23 (SEQ ID NOs: 1-336). Importantly, as the degree of infection was lowered, the number of individual cells decreased, but the signal intensity of the infected cells remained very high. Indeed, the signal intensity was high enough that signal was detected not only with a high-sensitivity 100× objective, but also with a relatively low power 20× objective. The ability to use much simpler and lower magnification microscopy to detect signals is important for designing a simple instrument capable of reading the output of the assay. A 5 minute hybridization time was used to perform the experiments, demonstrating that RNA FISH can rapidly detect influenza viral RNA,

To provide RNA FISH-based diagnostic readout of influenza infection, various sets of probes for influenza detection can be used. As is known to the skilled person bioinformatics can be used to design probes with minimal off-target binding. Testing of different RNA strands in infected cells (both vRNA and viral mRNA) can be used to determine which ones provide robust influenza detection. The development of image analysis algorithms can also increase robust and reliable determination of infection. It is expected that the use of cell-culture systems models many aspects of clinical use.

Hybridization, fixation and wash conditions can be altered to increase assay times. The data in other cell types showed that hybridization times were reduced from the standard 12-16 hours to as little as 30 seconds with no reduction in signal quality. Important modifications were methanol fixation and small hybridization volumes with high probe concentrations. As a demonstration that rapid hybridization times work for influenza detection, the results in FIG. 8 were obtained with 5 minute hybridizations. Additional results with influenza showed that these times can be reduced to 10 seconds without any loss in signal intensity (FIG. 9). Previous experiments with other mRNA targets show that there can be a compromise between probe concentration and hybridization time. Hybridization time and reagent cost are further considerations in producing an influenza diagnostic.

Beyond hybridization, the procedure also involves a 10 minute fixation time and 3 minutes for subsequent washes. Overall assay time can be reduced by cutting both fixation and wash times while maintaining signal quality. For instance, in some experiments, a two minute fixation with methanol at room temperature produced equivalent single molecule RNA FISH signal compared to conventional formaldehyde fixation and 10 minute methanol fixation at −20° C. Data in other cell lines suggests that fixation times can be reduced to under one minute and wash times to less than 1.5 minutes in total. Together, these data indicate a lower limit of within 5 minutes, preferably under 3 minutes, for the accurate detection of influenza virus via RNA FISH. Thus, decreasing fixation time provides a significant reduction in the overall assay time, while maintaining signal quality.

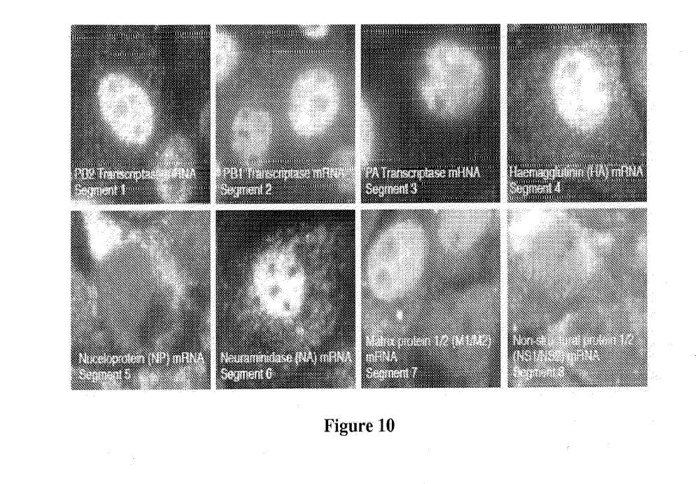

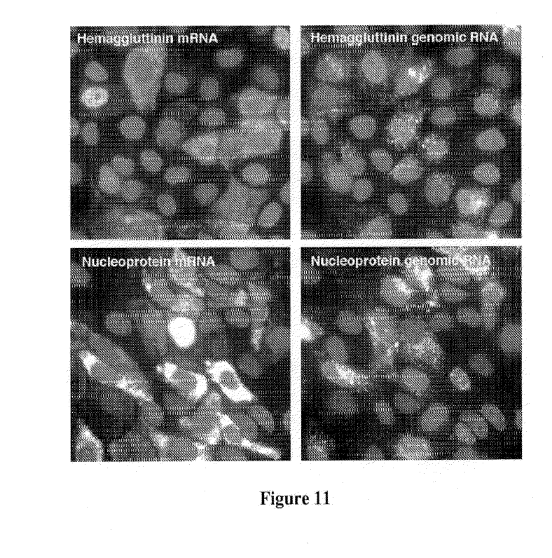

Rapid RNA FISH was performed on each of the viral segments individually. Using probe sets targeting the individual viral mRNAs from each viral segment, it was found that they exhibited a variety of localizations, with some nuclear, some cytoplasmic and some exhibiting both (FIG. 10). For instance, some were more nuclear or more cytoplasmic in localization, affecting how one computationally identifies the signal, and some were much more abundant than others, leading to more readily detectable fluorescent signal. These results demonstrate that it is feasible to target any of the segments and still generate suitable signal for detection. This is important because different segments exhibit different amounts of conservation between different strains, so this demonstrates flexibility to choose whatever segment is required for the assay. Viral genomic RNA has also been successfully targeted with equivalent signal strength (FIG. 11).

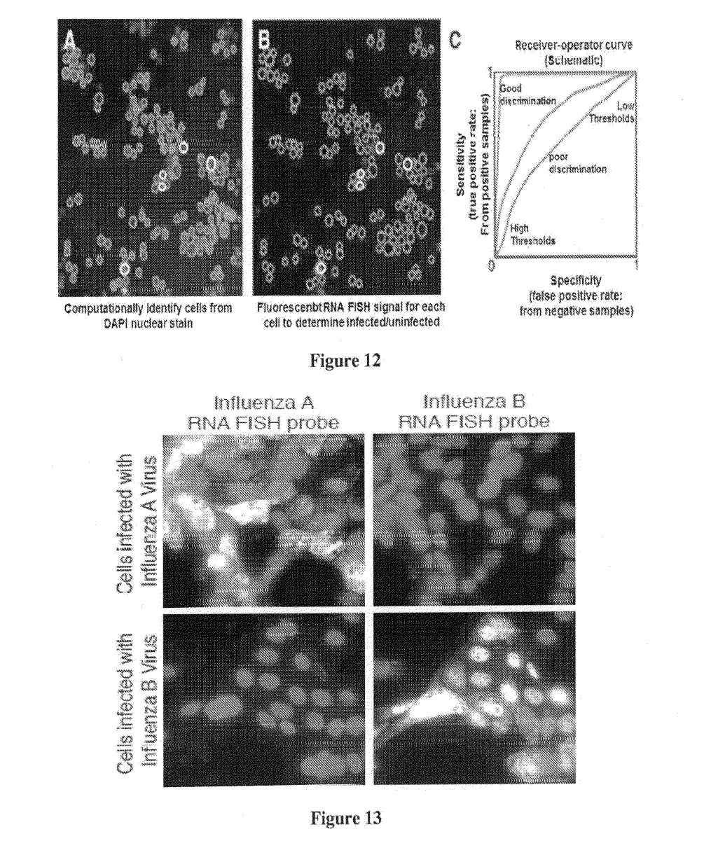

Another important aspect is the ability to automatically and computationally perform image analysis so as not to require the end user to read the slide. In one embodiment, probes maintaining a nuclear localization that generate the brightest possible signal are used. This approach facilitates identification of signal because of the presence of the DAPI nuclear counterstain. An algorithm has been developed that locates cell positions based on their DAPI signal based on Ostu's variance minimization method for thresholding and then computationally integrates fluorescence RNA FISH signal at the positions of those cells. The data indicate that the algorithm identified the vast majority of cells in the sample, and separated out positive from negative cells with a low rate of false positives and negatives. These rates were quantified by computing the receiver-operator curve using a variety of fluorescence intensity thresholds (FIG. 12). Time courses of viral infection can be used to see if the localization patterns of particular segments evolves during the viral life cycle. It is possible that such localization information in combination with the percentage of infected cells can be used to determine how long an infection has been present in a given patient sample. Probes that target the genomic vRNA provide an alternative set of probe targets and can be used to provide another signal for cross-validation. Multiple probe sets in the same cells can be detected by using multiple fluorophores using different wavelengths, thereby providing additional information. Thus, the signal intensity and localization patterns, can be used in a variety of ways to obtain a combination of conditions that maximizes fluorescent signal while maintaining optimal localization for computational identification and quantification.

An important consideration in the development of an image-based diagnostic is the ability to computationally identify positive cells from background. There exist very robust and reliable detection algorithms for identifying cells. In one embodiment cell nuclei can be detected with DAPI, total fluorescent intensity can be quantified in the nuclear region. Indeed very clear distinctions were observed, when comparing influenza infected cells to non-infected cells. Algorithms can be modified to aid in the analysis of the generally less clean signals in the clinical samples.

Several other viruses use proteins similar to those in influenza, and as such, it is critical to design probes that bind specifically only to influenza. An initial step is to take the sequences of a host of other respiratory viruses (such as respiratory syncytial virus) and use algorithms to ensure that influenza probe sequences will not bind to any of those other sequences. This can be validated experimentally by checking to make sure that the probes do not bind to cells infected with these other viruses.

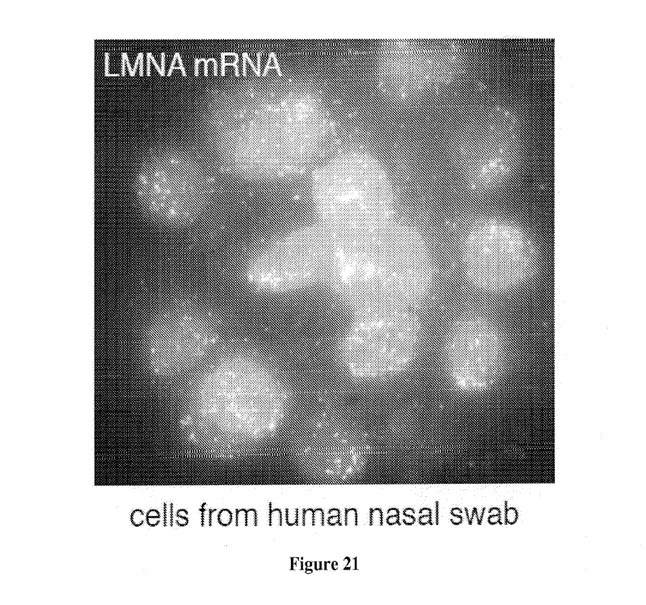

Data on clinical samples collected by a nasal swab (FIG. 21) showed that single Lamin A/C (LMNA) mRNA molecules can be accurately detected using the fluidic device and rapid RNA FISH of this invention. These results support the idea of a reliable and rapid detection of Influenza in clinics.

While the ability to detect signals in cell culture models of influenza infection is clearly established, there is always the potential for other sources of background in clinical samples that may confound detection. Experimentally, there are a number of ways to reduce background. Background may simply by decreased by further washing. Also, autofluorescence often is far more susceptible to photobleaching than the organic dyes that are used for imaging, so a simple exposure to shorter wavelengths may reduce background (Billinton et al., Anal Biochem. 2001 Apr. 15; 291(2):175-97). Further, there are chemical exposures that may help quench autofluorescence. Computationally, one can isolate signals from regions of high background by using DAP1 to identify areas with cells and separate those from areas without cells. Also, one can use parameters like the shape of the fluorescent signal to separate true from false signals.

### Development and Validation of Probes for Detecting Particular Strains of Influenza

Probes were used for detecting particular strains of influenza. Algorithms were developed and applied to design probes for distinguishing particular strains of influenza by RNA FISH. The probes were evaluated in cell culture systems.

There are a large number of strains of influenza, and distinguishing them is important for any diagnostic because the standard of care differs for different strains (e.g., anti-virals are typically reserved for type A infections). Even within the broadest influenza A/B categorization, particular strains often differ in their virulence and susceptibility to different anti-virals (e.g., H1N1, H3N2). Moreover, individual point mutations can confer drug resistance, and as such also provide important clinical information.

RNA FISH is ideally suited for strain discrimination due to the ability to design and pool particular groups of oligonucleotides. RNA FISH displayed a high degree of specificity (Raj et al., Nature Methods. 2008; 5(10):877-9), and more recently a new probe design allowed the ability to discriminate single base differences (Levesque et al., Nature Methods. 2013; 10(9):865-7). These advantageous aspects of RNA FISH were used to design and validate probes that are specific to particular strains of influenza with little to no cross-reactivity. Bioinformatic tools can be used for subtype-specific probe design as well as the application of novel RNA FISH methods that allow the ability to discriminate single base mismatches.

In order to distinguish influenza subtypes in RNA FISH assay, oligonucleotides that bind exclusively to one particular subtype at the exclusion of all others were designed. With the proliferation of sequencing information now available for different strains, access to large, up to date databases of strain nucleotide sequences is available. Using these databases, bioinformatic tools can generate a pool of oligonucleotides that only target a particular strain's sequence at the exclusion of all other relevant strains. Such an algorithm has been designed that screens out all potential oligonucleotide probes that have more than a specified number of mismatches with other targets. For instance, it can exclude all sequences with more than 14 of 20 bases matching the wrong target, which has been found to be sufficient to prevent the probe from off-target binding. These algorithms can be run on a large number of clinically relevant subtypes of influenza to generate subtype-specific probe sets. If the criteria are too restrictive to allow for any probes, the mismatch discrimination parameters can be relaxed to obtain at probes for detection purposes. If the sequences are so similar as to make discrimination by this approach impossible, a single nucleotide variant (SNV) detection scheme can be employed, as described below.

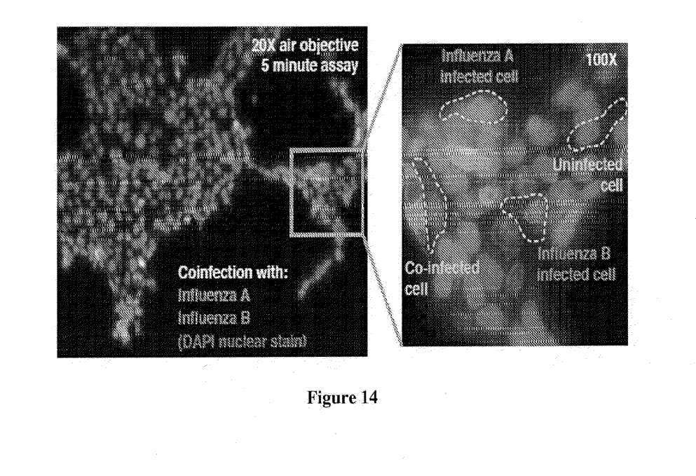

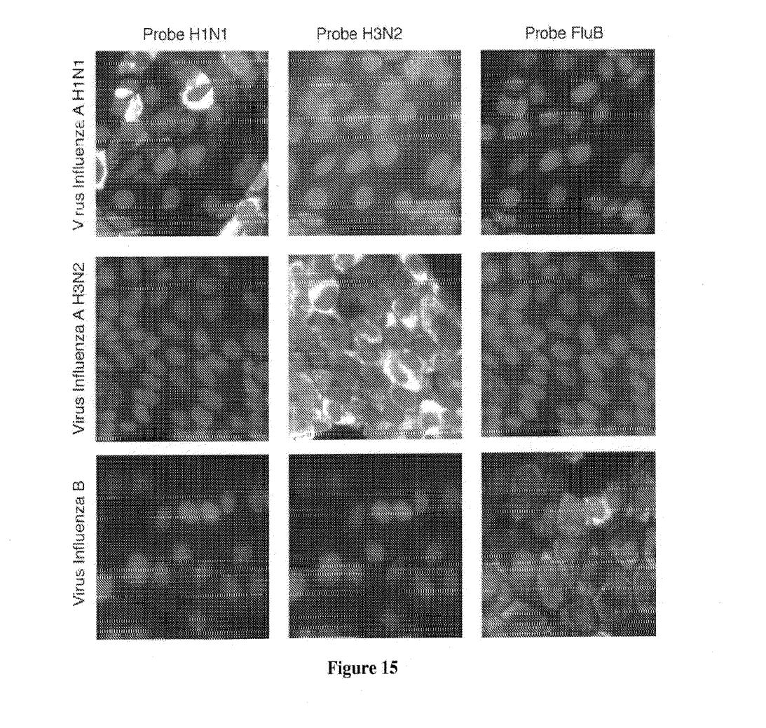

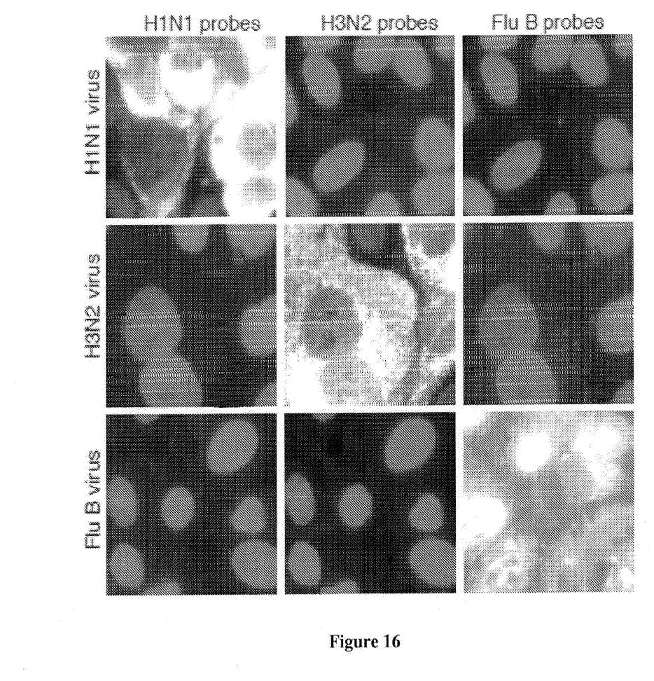

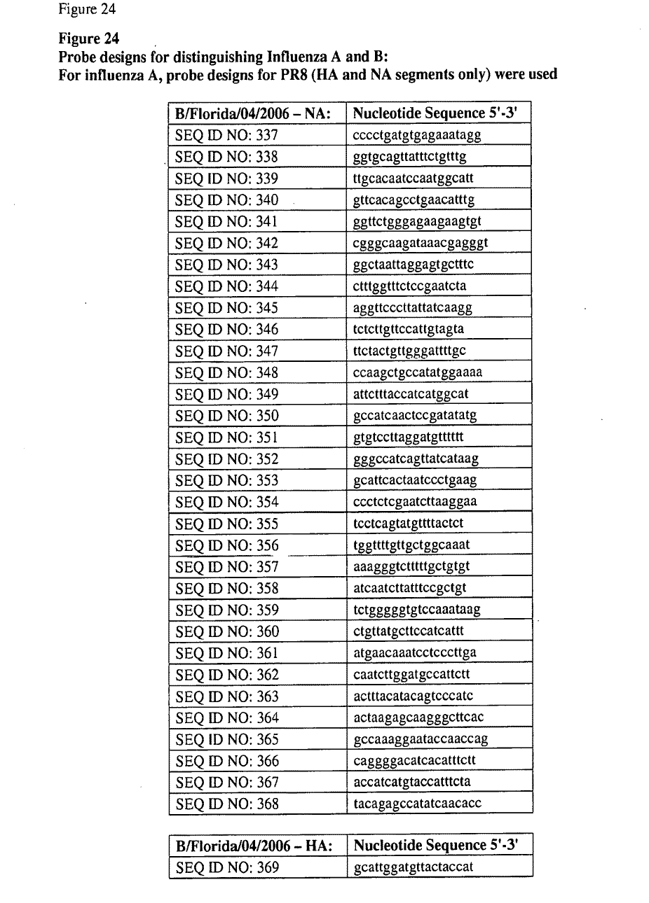

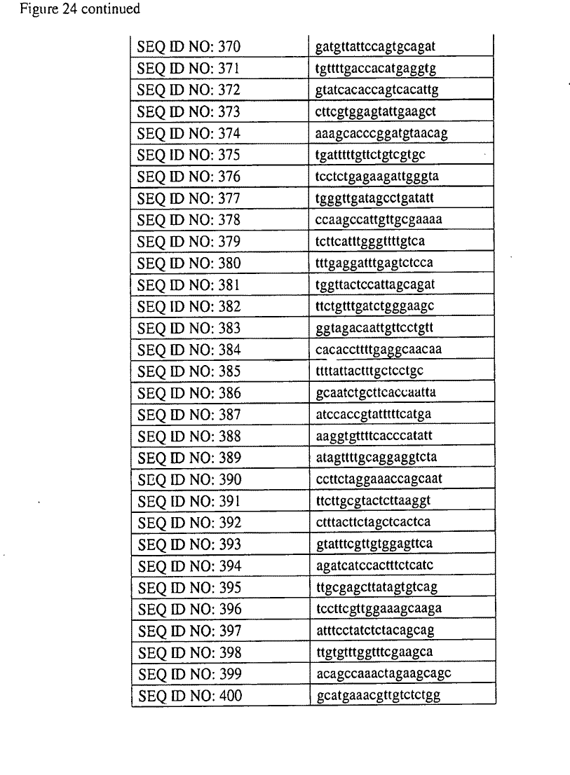

Influenza A and B have substantial sequence variation, but it is also important to distinguish subtypes of influenza A, such as H1N1 and H3N2, which have different clinical indications. These two strains differ primarily in the hemagglutinin and neuraminidase genes, providing a more stringent test of discriminatory capability. To see whether this approach would be able to discriminate at this level of resolution, oligonucleotide probe sets were designed that were predicted to be discriminatory based on base pairing. The specificity of probes generated by the algorithm were validated by first testing in MDCK cells infected with various strains of influenza. To demonstrate the feasibility of this approach, oligonucleotides specific to influenza A (PR8) and influenza B (B/Florida/04/2006), were generated. Probes targeting HA and NA segments of influenza A, as above, were used in the experiment. Probes targeting HA and NA segments of influenza B were used in the experiment and are depicted at FIG. 24 (SEQ ID NOs: 337-400). The probes were highly specific to the two strains with no cross-targeting, even when performing ultra-rapid RNA FISH and examining the results at high resolution (FIG. 13). To check whether these results held at lower spatial resolution, cells were coinfected with both strains. It was found that individual cells had RNA from either one or the other strain (and occasionally both) (FIG. 14), demonstrating the potential for a single ultra-rapid RNA FISH assay that can discriminate between two influenza strains. Additional oligonucleotide probes were made to distinguish further clinically relevant subtypes of influenza A, such as H1N1 and H3N2. Probes targeting influenza H1N1 and H3N2 bound with high affinity and produced high intensity signal in MDCK cells infected with H1N1, H3N2, respectively (FIG. 15). The probes yielded excellent specificity with virtually no cross-reactivity between probe sets when tested in a single assay (FIG. 16).

Additionally, probes targeting H1N1 and H3N2, produced no detectable cross-targeting with cells infected with other influenza sub-types. Ultimately, it would be advantageous to discriminate as many different subtypes as possible. Multi-color RNA FISH can be performed with probes simultaneously targeting genes that have regions of homology within most strains (such as nucleoprotein) in one color and specific subtypes of hemagglutinin and neuraminidase in other colors. Thus, a single assay can be used both to detect the presence of an infection and to subtype that infection.

There are particular variants that differ by just one or a few isolated single bases that have clinically significant differences in their behavior. For instance, before 2009, a significant number of influenza viruses had developed a single base mutation that conferred resistance to the anti-viral drug oseltamivir.

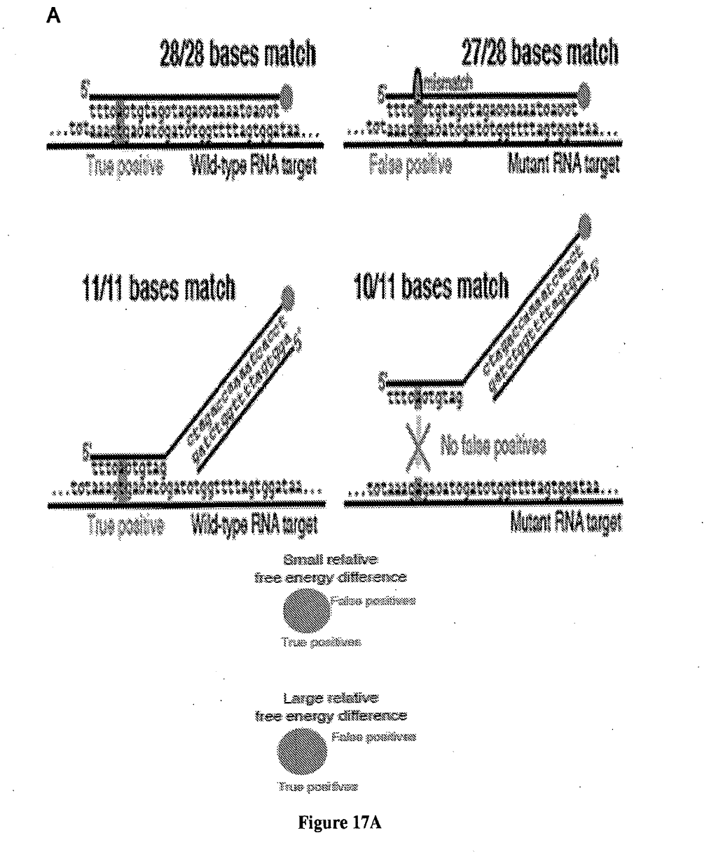

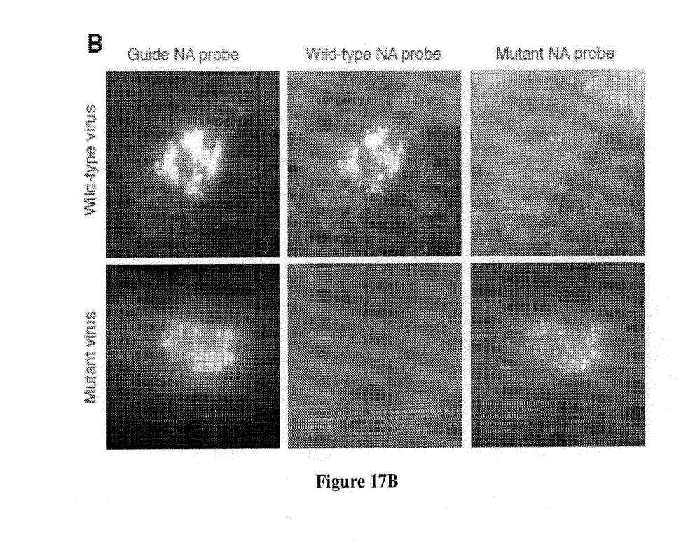

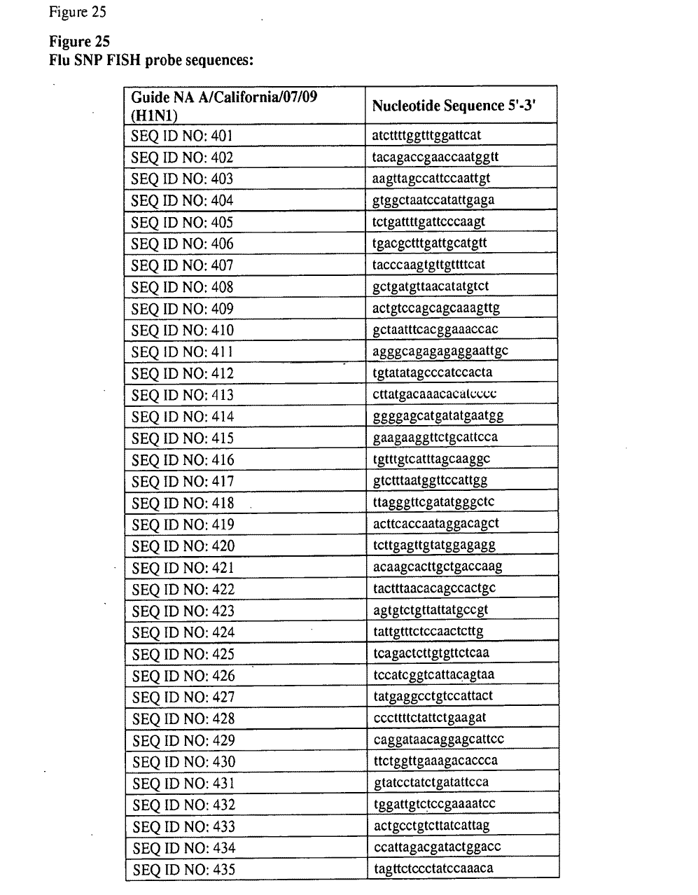

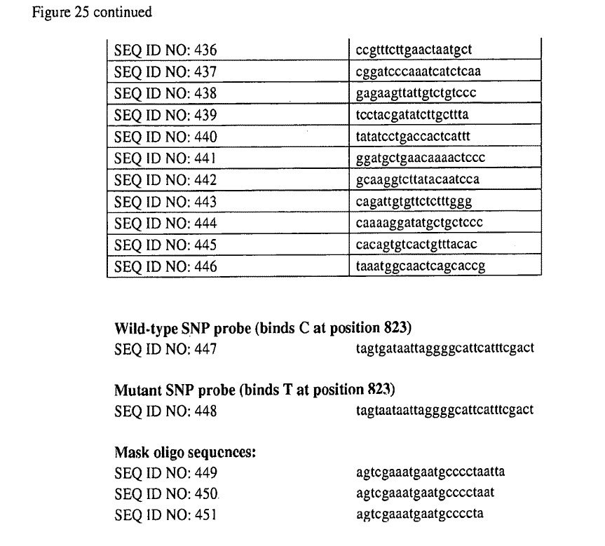

To detect such variants, an RNA FISH method (also compatible with very rapid hybridization (Shaffer et al., PLoS ONE. 2013; 8(9):e75120) was employed that enabled discrimination of single base differences with high accuracy. Conventionally designed RNA FISH probes (e.g., 20-mer oligonucleotide probes) are unable to detect single base differences with high specificity, owing to the fact that the difference in binding energy of a single base mismatch is not particularly large relative to the binding energy of an entire probe. To circumvent this difficulty (Levesque et al., Nature Methods. 2013; 10(9): 865-7) a “mask” oligonucleotide in combination with the basic probe oligonucleotide (Zhang et al., J Am Chem Soc. 2009; 131(47): 17303-14; Zhang et al., Nat Chem. 2012; 4(3): 208-14) was used in the RNA FISH assay. By using a “mask” oligonucleotide, the relative binding energy differences between a perfect match and single base mismatch oligonucleotides can be increased (FIG. 17A). Probes and oligonucleotides used in the experiment are depicted at FIG. 25 (SEQ ID NOs: 401-451).

To demonstrate the efficacy of this method, the A/California/07/2009 H1N1 strain containing a mutation (nucleotide position 823 C>T) in the neuraminidase gene conferring oseltamivir resistance was used to show probe specificity. The single mismatch was readily detected in cells infected with both wild-type and mutant influenza strains (FIG. 17B), using wild-type (SEQ ID NO: 447). and mutant probes (SEQ ID NO: 448), respectively. The SNV detection probes showed little to no cross-hybridization. In this experiment, overnight hybridization was used. However, it is expected that hybridization times can be reduced to around 5 minutes or less. Other variants that differ by only a single or a few bases can be discriminated from very similar strains in this manner. RT-PCR can be used to verify the nucleotide differences in strains and appropriate probes can be designed to discriminate those differences. As has been validated in cell culture, the probe methodology can be applied to clinical samples.

Thus, the ability to discriminate single base mismatches was demonstrated when the abundance of the target RNA was very high. However, when the target is at low abundance, it may be difficult to distinguish specific binding of the SNV detection probe from background probe binding. To deal with this issue, a guide probe approach (Levesque et al., Nature Methods. 2013 August; 10(9):865-7) can be used that enables one to distinguish only those probes that are bound to the actual target RNA. Results on cells infected with the A/California/07/2009 H1N1 strain mentioned above show that a method incorporating SNV and RNA FISH was capable of detecting individual RNA molecules with single base discrimination. In one embodiment, the hybridization time is reduced to under 5 minutes, especially for use with clinical samples. This methodology can extend the applicability of SNV detection into other diagnostic applications as well.

If the number of target RNA is small, an issue arises where high resolution microscopy is used for SNV detection. Thus, to facilitate detection image analysis may focus primarily on those cells with high RNA abundance (FIG. 17). Use of SNV in FISH may affect hybridization time, although SNV FISH has been demonstrated to work within 5 minutes of hybridization. If under other conditions, SNV FISH imaging time cannot be reduced to the same degree as with usual probe detection schemes, a sample can be tested on a device having separate SNV FISH and conventional RNA FISH assays, which provides a faster readout of a basic diagnosis followed by a more specific diagnosis a few minutes later.

### Development of a Device Capable of Performing Rapid Automated RNA FISH Assays

A microfluidic device was designed, fabricated, and tested that can rapidly and automatically perform RNA FISH on clinical samples. Bringing ultra-rapid RNA FISH to the point of care requires a simple device for performing the assay with minimal user training and hands-on time. In one embodiment, such a device is capable of automatically running RNA FISH assay on clinical isolates from a swab placed in a test tube. In one approach, the invention provides a simple, robust fluidic device that traps cells on a filter that holds cells in place for imaging while also allowing hybridization and wash solutions to rapidly pass over the cells (FIGS. 11A and 11B). This single-chip approach enables automation while minimizing sample loss and ensuring sensitivity, and miniaturization will enable to split the sample into multiple assays.

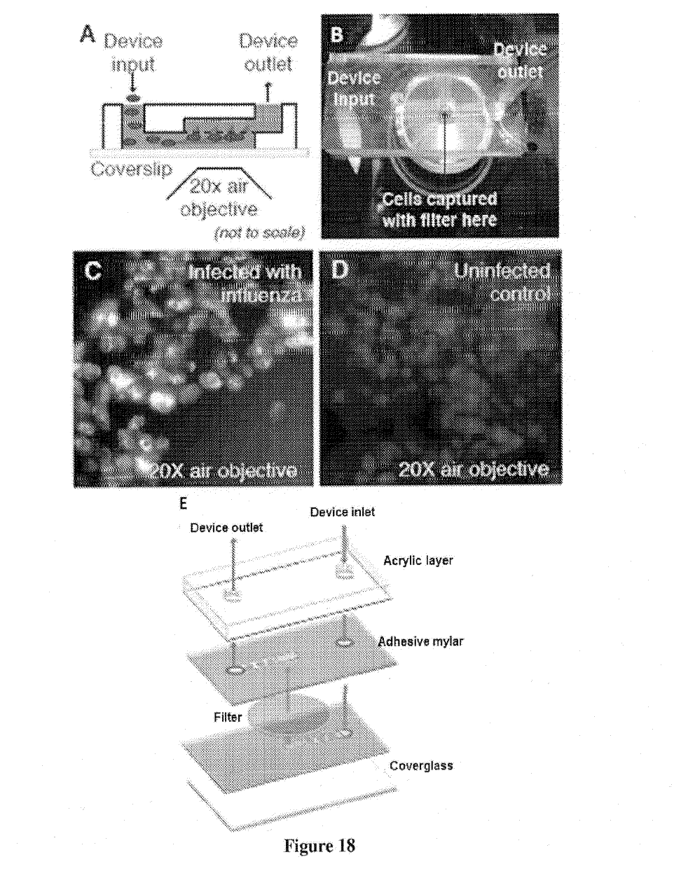

In one embodiment, the device comprises track-etched polycarbonate micropore filters integrated into laser-micromachined (Martin et al., Micromachining and Microfabrication. SPIE; 1998. p. 172-6) laminate sheet microfluidics (FIGS. 18A, 18B, 18E, and 18F). In contrast to conventional microfluidic devices, fluid flows vertically through the porous membrane allowing large flow rates (1 mL sample in less than 10 seconds) while keeping the capture rate high and the chip compact (Melaku et al., Advanced healthcare materials, 2014). This approach achieves a high capture rate of cells at fast flow rates, is robust to unprocessed samples, and can be manufactured at a low cost. In contrast to conventional microfluidic devices, fluid flows vertically through the porous membrane allowing large flow rates (1 mL sample in less than 10 seconds) while keeping the capture rate high and the chip compact (Noblitt et al., Anal Chem. 2007; 79(16): 6249-54; Chueh et al., Anal Chem. 2007; 79(9): 3504-8).

As an alternative to lithographic processing typically used to achieve single-cell capture, commercially available ion track-etched polycarbonate membranes (d=1 pm pore size) can be used. Unlike semiconductor processing, polycarbonate membranes can be fabricated with micro-scale pore sizes over large areas (A>10 cm²) for little cost (<5 ¢/cm²) (Whatman). The large density of micropores (ρ=10⁶ pores/cm²) reduces the risk of clogging from clinical and environmental samples, as the blockage of a few pores does not significantly change the behavior of the device. The filter traps the cells directly over a glass cover-slip, enabling imaging directly on-chip.

A prototype microchip was produced for performing RNA FISH on influenza cells. Testing of the device involved capturing both infected and uninfected cultured MDCK cells on the device (<5 seconds), performing rapid RNA FISH (5 minutes), and then washing (2 minutes) and imaging. It was found that the cells could be imaged after performing rapid RNA FISH on the microfluidic device using a 20× objective. This system achieved the same level of signal and specificity compared to conventional RNA FISH formats (FIGS. 18C and 18D). Background autofluorescence from the polycarbonate membrane and the laminate sheet microfluidics was insignificant compared to the RNA FISH labeled cells, enabling high contrast imaging. Importantly, the device was unaffected by the fixatives or other reagents used during the procedure, enabling the ultra-fast RNA FISH assay to be translated to use on the device without significant modifications.

Hybridization and washing times can be reduced to provide an assay that runs in about 3 minutes or less. Filter pore sizes can be selected to minimize clogging, and should not react with solvents used in the protocol. In one embodiment, the device has a capture rate ζ, >95% at a flow rate of φ>150 mL/hr (<10 seconds/1 mL), as measured by fluorescence imaging.

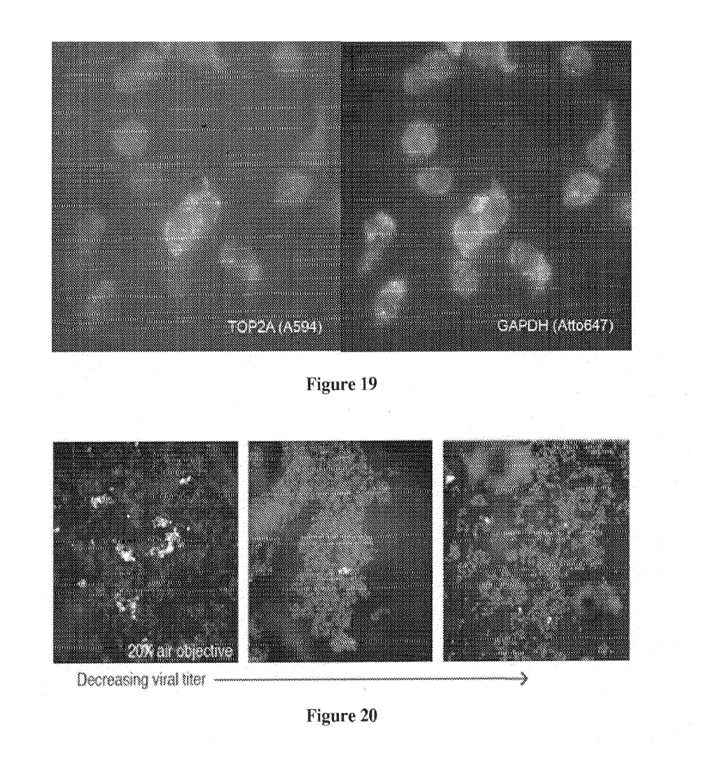

The Turbo FISH protocol was adapted for use for single molecule RNA FISH on the microfluidic device platform. Results using probes to TOP2A labeled with Alexa594 and GADPH labeled with Alexa647 in this assay showed detectable signal (FIG. 19). To perform this assay, a cell suspension was loaded into the device inlet reservoir. This suspension can contain either pre-fixed or unfixed cells. By pulling on the outlet syringe, the cell suspension flowed into the device and the cells were immobilized below the micropore filter. If the cells are unfixed, methanol (100%) can be flowed through the device to fix and permeabilize the cells. A pre-hybridization wash was performed by loading about 200 μL of wash buffer (containing 10% formamide and 2×SSC) into the device reservoir and pulling on the outlet syringe. After this wash, hybridization solution (10-50 μL) was placed into the reservoir and the syringe was pulled until the hybridization solution (containing 10% dextran sulfate, 10% formamide, 2×SSC, and 1 μL of each RNA FISH probe) covered the filter containing the cells. The microfluidic device was placed on a hotplate at 37° C. and incubated for 2-10 minutes to allow hybridization to occur. When the incubation time was over, the device was left on the hotplate and three wash steps were performed. For each washing step, wash buffer (200 μL) was loaded into the reservoir and this solution was pulled over the immobilized cells, the cells were incubated in wash buffer for 1 minute with each wash. Finally, the sample was removed from the hotplate and 2×SSC (200 μL) was loaded into the reservoir and this solution was pulled over the cells. At the end of the assay, 2×SSC (about 50-100 μL) was left in the reservoir to prevent the device from drying out during imaging. The signals obtained were equivalent to those detected on microscope slides, with the ability to easily detect rare infected cells (FIG. 20). At low magnification, the assay was sensitive enough to detect signal from single cells.

Cells were isolated from nasal swab from a healthy adult and fixed. A portion of the cells (10%) were run through a prototype of the fluidic device. (FIG. 21). Approximately ˜30,000 cells per swab were collected for analysis, indicating that a single swab contained enough cells to run at least 10 assays, thus enabling multiplexing.

Multiple RNA FISH assays can be integrated onto a single chip, enabling the multiplexed detection of several RNA FISH markers. This integration allows multiple virus-types to be to be rapidly screened concurrently in a practical clinical setting. To achieve this functionality, a microfluidic device was designed in which cells from the clinical sample are evenly split into four isolated regions for micropore based capture and hybridization (FIGS. 22A and 22B). In another study, a microfluidic geometry was designed in which cells from the clinical sample are evenly split into sixteen isolated regions for micropore based capture and hybridization. To evenly distribute the sample, a “shower head” geometry was implemented in which symmetric branching is used to split the flow evenly to sixteen 1 mm² holes above the micropore filter (FIG. 22C). Cells trapped in each of the individual trapping regions are exposed to a unique RNA FISH probe. Continuous negative pressure from the output ensures that no mixing occurs between the detection regions. Thus, the number of RNA FISH experiments on a single chip can be increased beyond 16 by increasing the number of branches n in the symmetric branching geometry. Each device can include N=2ⁿ isolated detection regions with the theoretical upper limit set only by the number of cells obtained from the patient. Such a device would enable to run panels of assays simultaneously, each detecting specific variants or even different viruses in the same chip. To test the capability of performing multiplexed assays on a chip with clinical isolates, a chip that can perform sixteen independent RNA FISH assays has been made and is being tested.

A fundamental challenge for expanding from a single assay on a chip to larger numbers of parallel assays, is that use of the chip becomes impractical if the user has to supply too many inputs. To address this issue, a chip has been designed that includes all of the reagents for the RNA FISH assay, including the RNA FISH probes and the washing solution, preloaded for easy use. To accomplish this aim, a design strategy was adopted that was implemented on a multiplexed ELISA-based chip for point-of-care diagnosis of sexually transmitted disease in resource limited settings (Chin et al., Nat Med. 2011; 17(8): 1015-9). In this design, metered quantities of RNA FISH reagents are preloaded into tubes separated by air bubble spacers while loading the nasal swab/aspirate into a separate reservoir. The sample is loaded and concentrated, and the RNA FISH reagents are drawn into the device using a single syringe held at negative pressure. To remove the air bubble spacers, a micro-scale bubble trap (Sung et al., Biomed Microdevices. 2009; 11(4):731-8) prevents the bubbles from interfering with imaging or from releasing the cells from the micropore filter. This design strategy requires no moving parts, electricity, or external instrumentation. In one embodiment, the assay is performed in under 5 minutes, preferably 3 minutes, with low cell loss ζ>95%, insignificant mixing between the assays, and/or high contrast imaging of the cells. The ease of use can be validated by testing their use with medical personnel and quantifying user variability.

### Rapid and Quantitative Detection Using Turbo RNA FISH

A protocol that enables rapid and quantitative detection of RNA targets via RNA FISH is described herein. It was found that alcohol-based fixatives provide the necessary probe accessibility for rapid hybridization via increased probe concentration, potentially allowing for hybridizations in as little as 30 seconds. The experiments show that there is a straightforward tradeoff between concentration of probe and the speed of hybridization. It was found that increasing probe concentration by 20× compared to a typical overnight protocol yields reliable RNA FISH results after just 5 minutes of hybridization. At first glance, this increased probe concentration may not seem economically viable, considering the increased use of probes (which are the most costly reagent in the RNA FISH protocol). However, because of the decreased time for drying, the protocol of the present invention uses roughly 10 fold less hybridization solution for the hybridization itself, greatly mitigating such concerns. The ultimate choice of how much probe to use and how fast to drive the reaction may depend on the specifics of the application at hand. In some cases, getting a hybridization time of 5-10 minutes may be acceptable, in which case the use of large concentrations of probe may not be needed. However, in some situations, such as during a surgical procedure, the decreased hybridization times may be a benefit that outweighs the cost of increased probe usage.

Of course, even with rapid hybridizations, the issue of the imaging time itself has not been addressed. Typically, image acquisition may require taking image stacks from multiple positions on the slide to obtain enough cells worth of image data to make statistically significant claims about differences in gene expression. Currently, doing so could take on the order of 10-20 minutes per condition. However, it is believed that technical advances can reduce the time required for both image acquisition and analysis by at least an order of magnitude. In such a case, comparing gene expression in two samples in well under 30 minutes from living cells to quantified data can be envisioned.

A comparison to other methods such as Reverse Transcription real-time polymerase chain reaction (RT-qPCR) is now discussed. RT-qPCR is widely considered to be the most accurate method for quantifying gene expression to date. It has many benefits, including high dynamic range, low cost per reaction, and the ability to parallelize in 96-well plate format. The qPCR itself usually takes on the order of 1-2 hours to complete, but if both RNA extraction and setup time is included, the total time required is probably closer to around 3-4 hours. These extra steps also increase the cost of the experiment as well. With rapid hybridization, RNA FISH can compete favorably with RT-qPCR on most counts. With respect to quantification, the method provides accurate, absolute counts of gene expression of 3 to 5 genes in individual cells without the explicit need for normalization. Since RNA FISH is a direct detection scheme without any amplification, even small fold-changes can be detected with high precision (Raj et al., 2008, Nat Methods 5:877-879), differences that would be hard to measure accurately with RT-qPCR, at least not without a large number of replicates. The cost per reaction is probably dominated by the cost of the probe, which is currently around $300-$600 per probe set for 10,000 hybridizations ($0.06 per reaction) and is thus comparable to a molecular beacon or Taq-man RT-qPCR probe.

Of course, costs of labor, equipment and other reagents are variables that are hard to predict, but will be of the same order of magnitude, although it is noted that the labor required for RNA FISH is probably lower, whereas the cost of an automated microscope may be higher than most qPCR machines. Both the accuracy and cost comparisons to RT-qPCR are valid even with overnight RNA FISH. The time required for previous iterations of RNA FISH, however, was considerably longer than for RT-qPCR, and the method of the present invention alleviates that discrepancy. If the expression of only a few genes is compared in a few conditions, then the method of the present invention is unequivocally several times faster than RT-qPCR, especially when RNA extraction and setup time is included. For analyzing larger numbers of genes in parallel, though, the imaging time will become a factor. If one assumes 5-10 minutes per condition and triplex RNA detection, then analyzing, say, 20-30 genes could require up to 2 hours. With advances in high throughput imaging, this time can be reduced by an order of magnitude, thus further increasing the speed advantages.

Another major advantage of RNA FISH is that it also provides single cell information, something that is much more difficult to obtain with single cell RT-qPCR approaches. This enables one to measure variability in gene expression from cell to cell. Since the measurements yield absolute numbers of RNA, the measurements do not necessarily require normalization to an internal control (such as GAPDH), although such an analysis can be performed through multiplexing. Normalization can be difficult to perform with RT-qPCR approaches, since all the material is typically used for a single qPCR reaction, leaving none for further normalization.

Furthermore, RNA FISH also provides spatial information on the localization of RNA. Such information is important both for examining differences from cell to cell within a tissue and even subcellular spatial localization. In tissues, particular cells can be easily identified by labeling RNA specific to those cells with one color and then looking at the gene of interest in another color. Subcellular information can be of particular importance for RNA that localize to particular regions of the cell, such as many non-coding RNA, in which case RNA FISH can reveal much about its behavior.

It is also demonstrated herein that one can perform iceFISH and SNP FISH to visualize chromosomes and single base changes, respectively, with rapid hybridization. Such techniques could be useful for rapidly diagnosing chromosomal abnormalities and for rapid genotyping of particular single nucleotide variants.

In summary, the method for rapid hybridization of the present invention results in orders of magnitude improvements in hybridization time for single molecule RNA FISH, enabling a new set of high throughput and rapid diagnostic applications.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for a rapid detection of a target nucleic acid in a sample, the method comprising:
contacting the sample with a non-crosslinking fixative, thereby producing a fixed sample;
contacting the fixed sample with a hybridization solution, the hybridization solution comprising at least one labeled probe which hybridizes to a region of the target nucleic acid, wherein the detection of the labeled probe indicates the detection of the target nucleic acid in the sample; and
wherein the detection method is conducted in less than 5 hours.

2. The method of claim 1, wherein the non-crosslinking fixative comprises an alcohol selected from the group consisting of ethanol and methanol.

3. The method of claim 1, wherein the target nucleic acid is RNA.

4. The method of claim 1, wherein the target nucleic acid comprises a mutational variant.

5. The method of claim 1, wherein the sample is obtained from a mammal.

6. The method of claim 5, wherein the mammal is a human.

7. The method of claim 1, wherein the one labeled probe concentration is about 0.1 mM to about 20 mM.

8. The method of claim 1, wherein the one labeled probe concentration is about 3 mM to about 4 mM.

9. The method of claim 1, wherein the sample is contacted with the non-crosslinking fixative for about 10 minutes.

10. The method of claim 1, wherein the sample is contacted with the non-crosslinking fixative for about 2 minutes.

11. The method of claim 1, wherein the fixed sample is contacted with the hybridization solution for less than about 2 hours.

12. The method of claim 1, wherein the fixed sample is contacted with the hybridization solution for less than about 1 hour.

13. The method of claim 1, wherein the fixed sample is contacted with the hybridization solution for less than about 10 minutes.

14. The method of claim 1, wherein the fixed sample is contacted with the hybridization solution for less than about 1 minutes.

15. The method of claim 1, wherein the fixed sample is contacted with the hybridization solution for less than about 30 seconds.

16. The method of claim 1, wherein the detection method is conducted in less than 2 hours.

17. The method of claim 1, wherein the detection method is conducted in less than 10 minutes.

18. The method of claim 1, wherein the detection method is conducted in less than 5 minutes.

19. The method of claim 1, wherein the at least one probe is labeled with a fluorophore.

20. The method of claim 19, further comprising detecting the fluorophore.

21. The method of claim 3, wherein the RNA is selected from the group consisting of messenger RNA, intronic RNA, exonic RNA, and non-coding RNA.

22. The method of claim 1, wherein the method is used to quantify the presence of the target nucleic acid.

23. The method of claim 1, wherein the method is used to investigate chromosome structure and transcriptional activity.

24. The method of claim 1, wherein the method is used to identify a mutation in the target nucleic acid.

25. The method of claim 1, wherein the method is used in high-throughput screening.

26. The method of claim 1, wherein the method is used in rapid diagnostics.

27. The method of claim 1, wherein the target nucleic acid comprises an viral nucleic acid sequence.

28. The method of claim 27, wherein the viral nucleic acid comprises an influenza nucleic acid sequence.

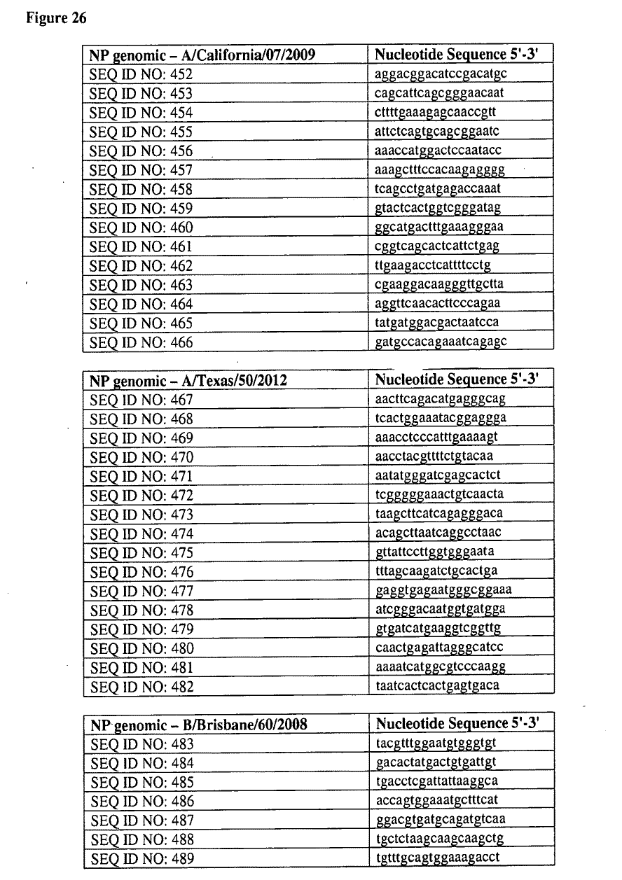

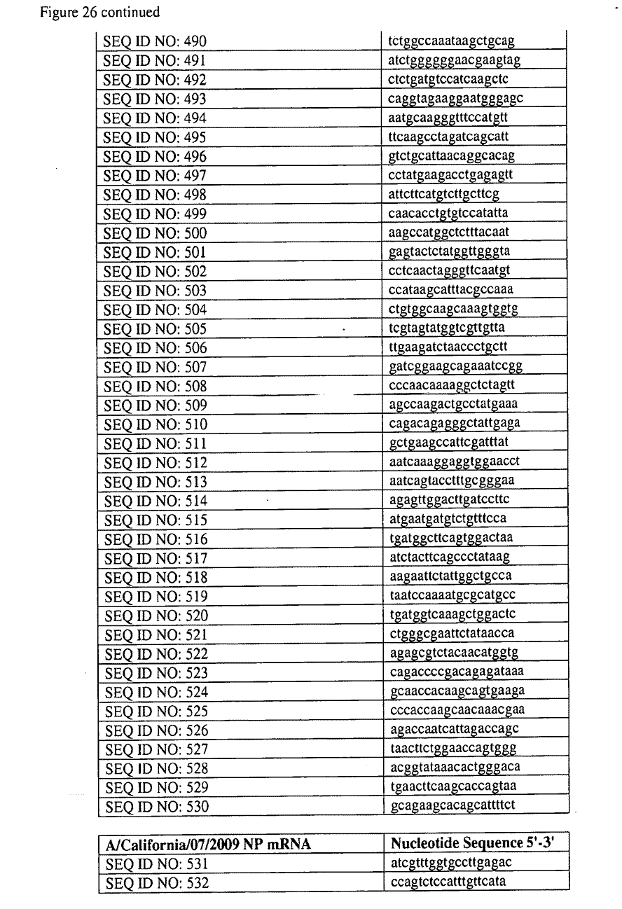

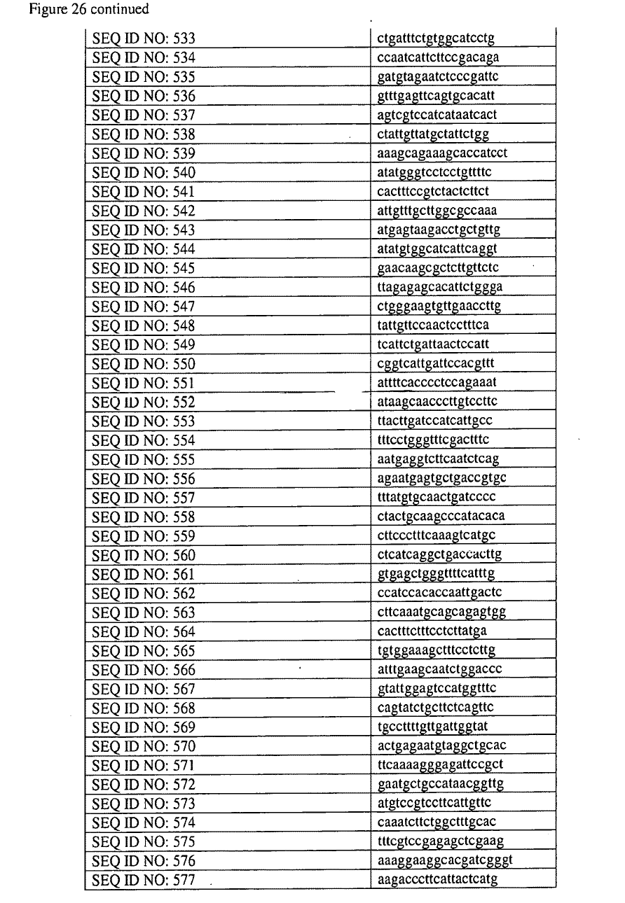

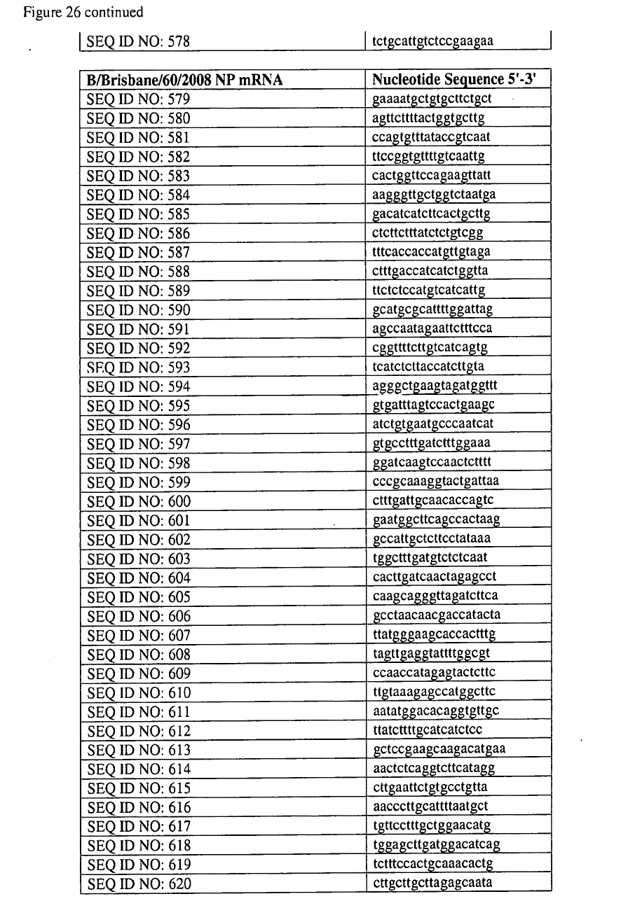

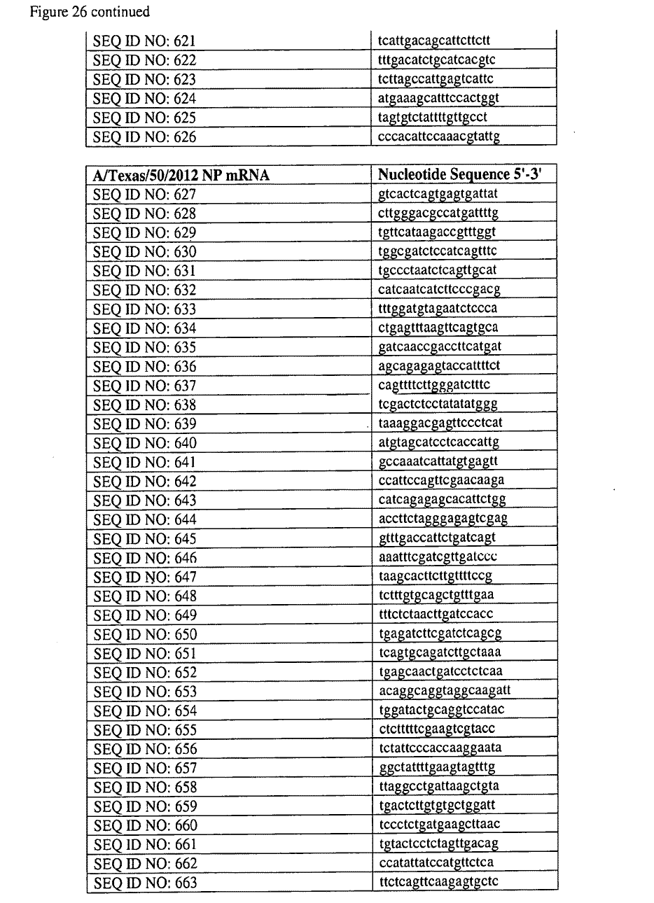

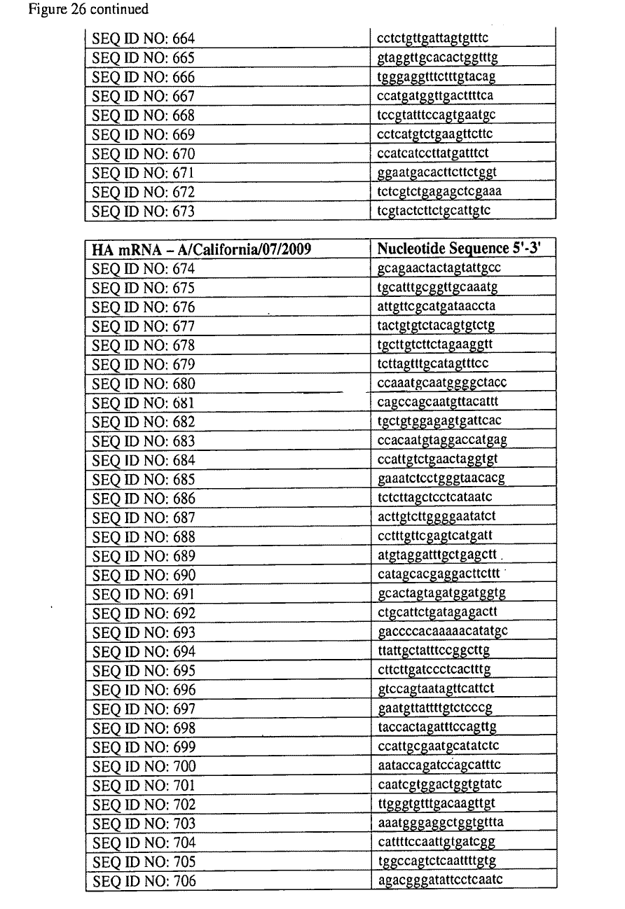

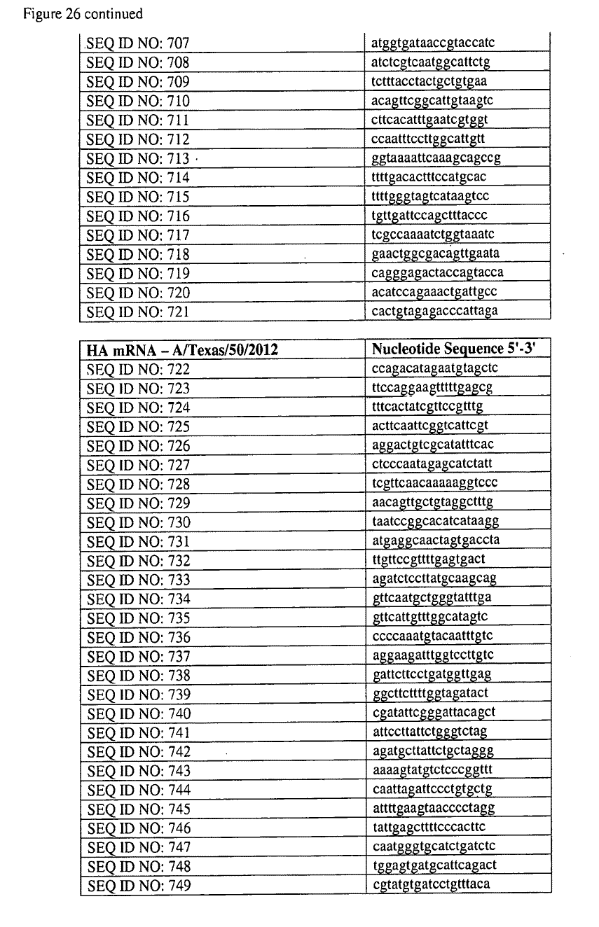

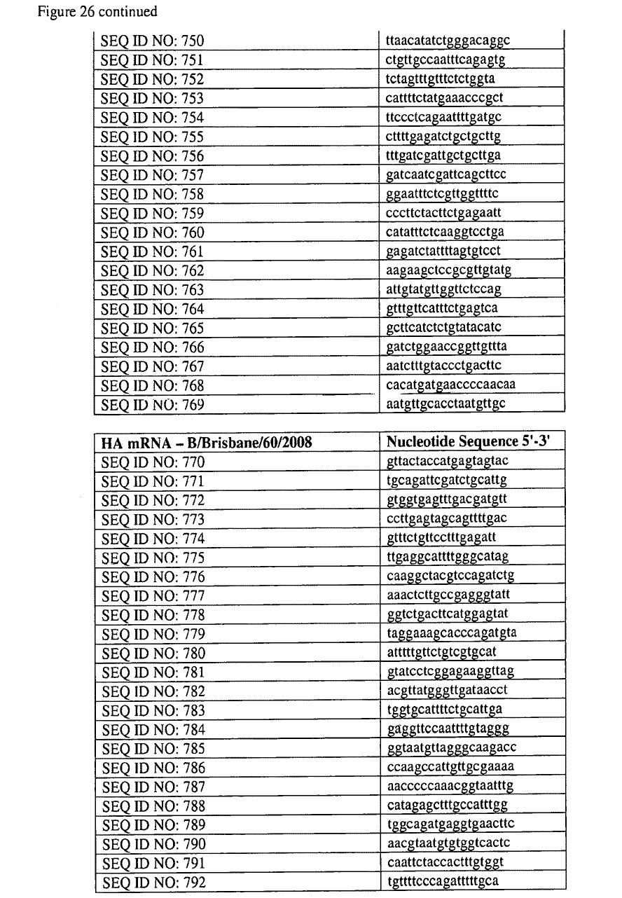

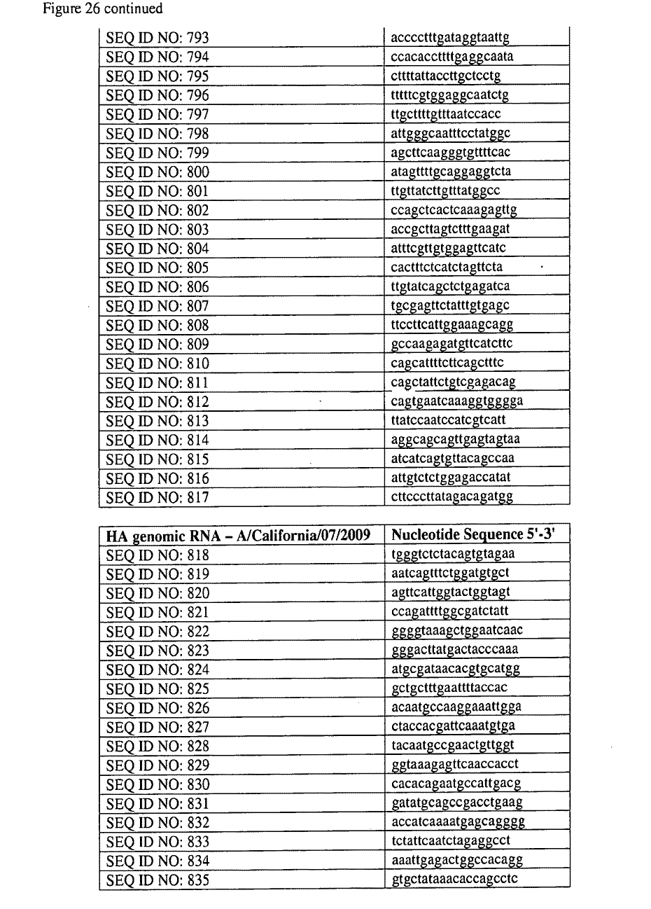

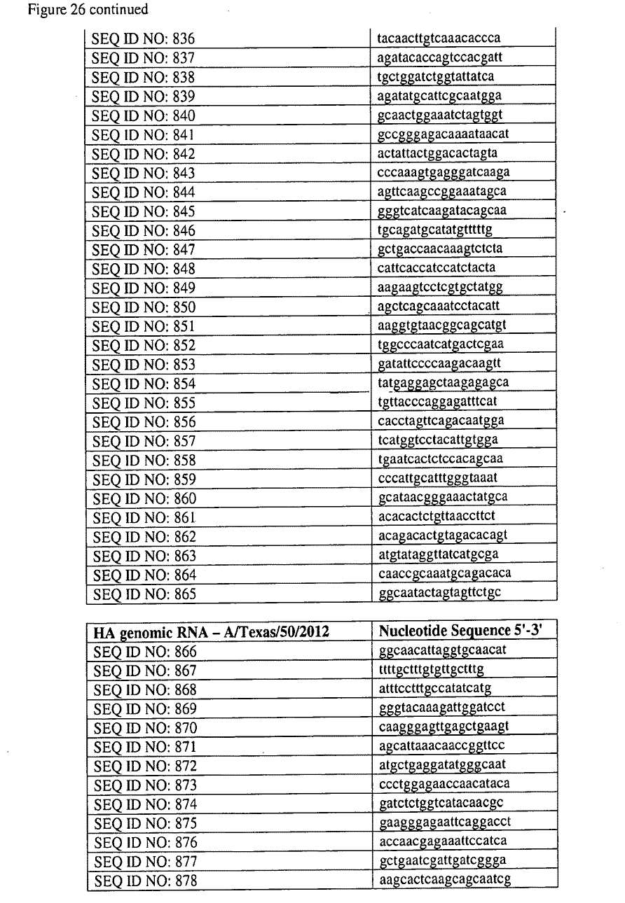

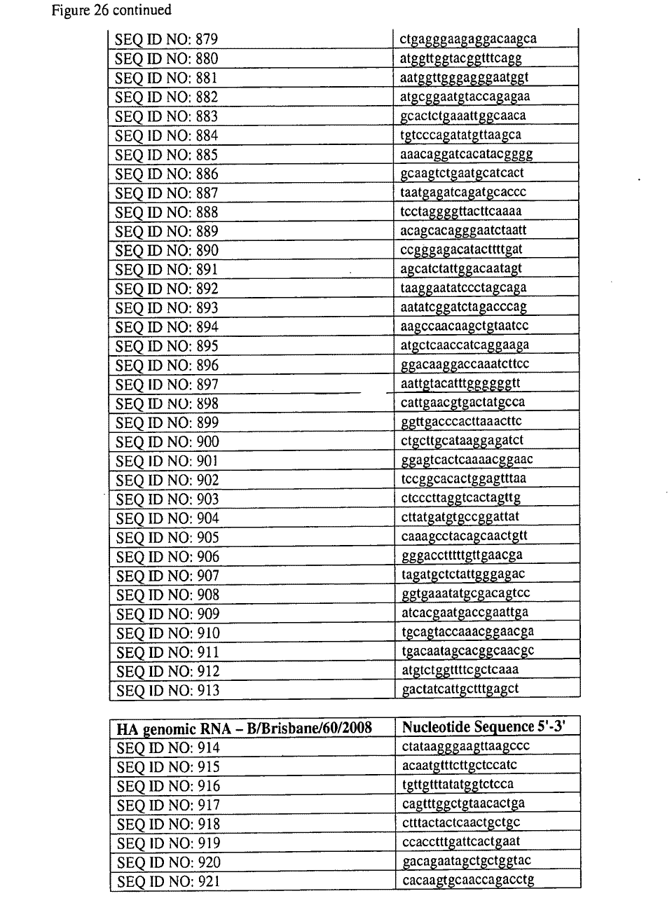

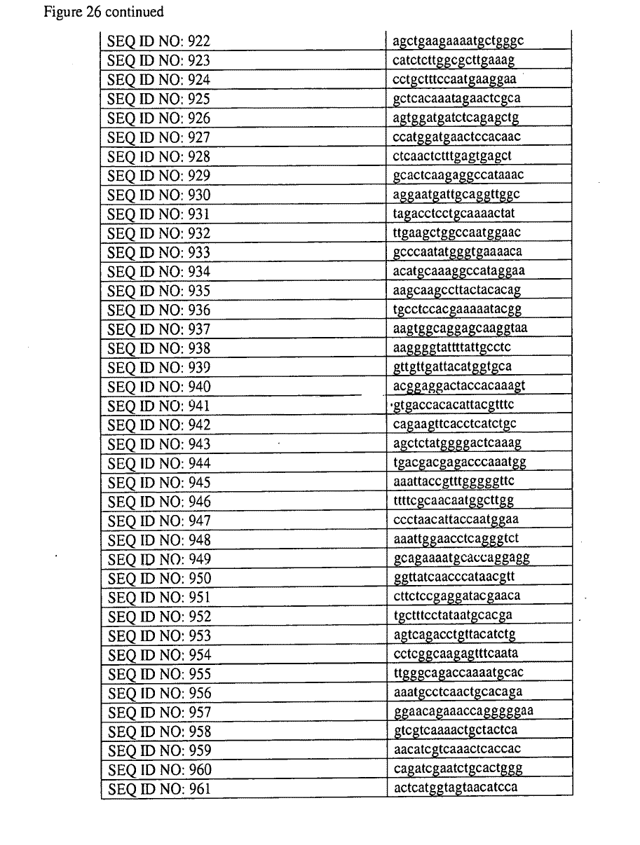

29. The method of claim 1 wherein, the probe is one or more oligonucleotides depicted at FIGS. 23, 24, 35, and 26 (SEQ ID NOs:1-961).

30. The method of claim 1, comprising the use of a microfluidics device, the microfluidics device comprising one or more openings in fluid communication with one or more liquid reservoirs or wells.

31. The method of claim 30, wherein the microfluidics device is optically transparent.

32. The method of claim 30, wherein the sample is introduced into a liquid reservoir or well of the microfluidics device.

33. The method of claim 30, comprising introducing a fluid into the liquid reservoir or well.

34. The method of claim 30, wherein the fluid comprises one or more of a labeled probe, a non-crosslinking fixative, or a buffer.

35. The method claim 30, wherein the microfluidics device comprises the labeled probe preloaded into a liquid reservoir or well.

36. A method for a rapid detection of an influenza target nucleic acid in a cell from a biological sample, the method comprising:
contacting a cell of the sample with a non-crosslinking fixative, thereby producing a fixed cell;
contacting the fixed cell with a hybridization solution, the hybridization solution comprising at least one labeled probe which hybridizes to a region of the influenza target nucleic acid, wherein the detection of the labeled probe indicates the detection of the influenza target nucleic acid in the cell; and
wherein the detection method is conducted in less than 5 hours.

37. The method of claim 36, wherein the non-crosslinking fixative comprises an alcohol selected from the group consisting of ethanol and methanol.

38. The method of claim 36, wherein the target nucleic acid is RNA.

39. The method of claim 36, wherein the target nucleic acid comprises a mutational variant.

40. The method of claim 36, wherein the sample is obtained from a mammal.

41. The method of claim 40, wherein the mammal is a human.

42. The method of claim 36, wherein the one labeled probe concentration is about 0.1 mM to about 20 mM

43. The method of claim 36, wherein the one labeled probe concentration is about 3 mM to about 4 mM

44. The method of claim 36, wherein the labeled probe is one or more oligonucleotides depicted at FIGS. 23, 24, 35, and 26 (SEQ ID NOs:1-961).

45. The method of claim 36, wherein the sample is contacted with the non-crosslinking fixative for about 10 minutes.

46. The method of claim 36, wherein the sample is contacted with the non-crosslinking fixative for about 2 minutes.

47. The method of claim 36, wherein the fixed sample is contacted with the hybridization solution for less than about 2 hours.

48. The method of claim 36, wherein the fixed sample is contacted with the hybridization solution for less than about 1 hour.

49. The method of claim 36, wherein the fixed sample is contacted with the hybridization solution for less than about 10 minutes.

50. The method of claim 36, wherein the fixed sample is contacted with the hybridization solution for less than about 1 minutes.

51. The method of claim 36, wherein the fixed sample is contacted with the hybridization solution for less than about 30 seconds.

52. The method of claim 36, wherein the detection method is conducted in less than 2 hours.

53. The method of claim 36, wherein the detection method is conducted in less than 10 minutes.

54. The method of claim 36, wherein the detection method is conducted in less than 5 minutes.

55. The method of claim 36, wherein the at least one probe is labeled with a fluorophore.

56. The method of claim 55, further comprising detecting the fluorophore.

57. The method of claim 36, wherein the RNA is selected from the group consisting of messenger RNA, intronic RNA, exonic RNA, and non-coding RNA.

58. The method of claim 36, wherein the method is used to quantify the presence of the target nucleic acid.

59. The method of claim 36, wherein the method is used to investigate chromosome structure and transcriptional activity.

60. The method of claim 36, wherein the method is used to identify a mutation in the target nucleic acid.

61. The method of claim 36, wherein the method is used in high-throughput screening.

62. The method of claim 36, wherein the method is used in rapid diagnostics.

63. The method of claim 36, wherein the target nucleic acid comprises an viral nucleic acid sequence.

64. The method of claim 63, wherein the viral nucleic acid comprises an influenza nucleic acid sequence.

65. The method of claim 36, comprising the use of a microfluidics device, the microfluidics device comprising one or more openings in fluid communication with one or more liquid reservoirs or wells.

66. The method of claim 65, wherein the microfluidics device is optically transparent.

67. The method of claim 65, wherein the sample is introduced into a liquid reservoir or well of the microfluidics device.

68. The method of claim 65, comprising introducing a fluid into the liquid reservoir or well.

69. The method of claim 65, wherein the fluid comprises one or more of a labeled probe, a non-crosslinking fixative, or a buffer.

70. The method claim 65, wherein the microfluidics device comprises the labeled probe preloaded into a liquid reservoir or well.

71. A fluidic device for detection of influenza in a sample, the fluidic device comprising one or more openings in fluid communication with one or more liquid reservoirs or wells, wherein the liquid reservoirs or wells comprise one or more labeled probes depicted at FIGS. 23, 24, 35, and 26 (SEQ ID NOs:1-961).

72. A kit comprising at set of probes for detection of nucleic acids in a sample and instructions for use thereof.


SEQUENCE LISTING (961 sequences)

SEQ ID NO: 1 - DNA, 20 bp, Artificial Sequence
  Chemically synthetized sequence
cttaacaggaccagtaggtt

SEQ ID NO: 2 - DNA, 20 bp, Artificial Sequence
  Chemically synthetized sequence
atattgtgtctgcatctgca

SEQ ID NO: 3 - DNA, 20 bp, Artificial Sequence
  Chemically synthetized sequence
tgaattgttcgcatggtagc

SEQ ID NO: 4 - DNA, 20 bp, Artificial Sequence
  Chemically synthetized sequence
gtactgtgtcaacagtgtcg

SEQ ID NO: 5 - DNA, 20 bp, Artificial Sequence
  Chemically synthetized sequence
tgtgtcactgtcacattctt

SEQ ID NO: 6 - DNA, 20 bp, Artificial Sequence
  Chemically synthetized sequence
gtcttcgagcaggttaacag

SEQ ID NO: 7 - DNA, 20 bp, Artificial Sequence
  Chemically synthetized sequence
tacatagttttccgttgtgg

SEQ ID NO: 8 - DNA, 20 bp, Artificial Sequence
  Chemically synthetized sequence
agtggggctattccttttaa

SEQ ID NO: 9 - DNA, 20 bp, Artificial Sequence
  Chemically synthetized sequence
gatgttacatttccccaatt

SEQ ID NO: 10 - DNA, 20 bp, Artificial Sequence
  Chemically synthetized sequence
cattctgggtttcccaagag

SEQ ID NO: 11 - DNA, 20 bp, Artificial Sequence
  Chemically synthetized sequence
tgatctcactggaagcagtg

SEQ ID NO: 12 - DNA, 20 bp, Artificial Sequence
  Chemically synthetized sequence
attccattctcagagtttgg

SEQ ID NO: 13 - DNA, 20 bp, Artificial Sequence
  Chemically synthetized sequence
gctcctcatagtcgatgaaa

SEQ ID NO: 14 - DNA, 20 bp, Artificial Sequence
  Chemically synthetized sequence
tgacactgagctcaattgct

SEQ ID NO: 15 - DNA, 20 bp, Artificial Sequence
  Chemically synthetized sequence
ccatgagctttctttgggaa

SEQ ID NO: 16 - DNA, 20 bp, Artificial Sequence
  Chemically synthetized sequence
gttactccgtttgtgttgtg

SEQ ID NO: 17 - DNA, 20 bp, Artificial Sequence
  Chemically synthetized sequence
taaaaactgcttttcccctc

SEQ ID NO: 18 - DNA, 20 bp, Artificial Sequence
  Chemically synthetized sequence
cgtcagccatagcaaatttc

SEQ ID NO: 19 - DNA, 20 bp, Artificial Sequence
  Chemically synthetized sequence
tgttgttccttactgttagg

SEQ ID NO: 20 - DNA, 20 bp, Artificial Sequence
  Chemically synthetized sequence
ctatttccggggtaaatctc

SEQ ID NO: 21 - DNA, 20 bp, Artificial Sequence
  Chemically synthetized sequence
cttgatctcttactttgggt

SEQ ID NO: 22 - DNA, 20 bp, Artificial Sequence
  Chemically synthetized sequence
tccagtaatagttcatcctc

SEQ ID NO: 23 - DNA, 20 bp, Artificial Sequence
  Chemically synthetized sequence
attagatttccatttgcctc

SEQ ID NO: 24 - DNA, 20 bp, Artificial Sequence
  Chemically synthetized sequence
tctactcagtgcgaaagcat

SEQ ID NO: 25 - DNA, 20 bp, Artificial Sequence
  Chemically synthetized sequence
tgatgatgccggacccaaag

SEQ ID NO: 26 - DNA, 20 bp, Artificial Sequence
  Chemically synthetized sequence
tcatgcattgatgcgtttga

SEQ ID NO: 27 - DNA, 20 bp, Artificial Sequence
  Chemically synthetized sequence
gtgtttgacacttcgtgtta

SEQ ID NO: 28 - DNA, 20 bp, Artificial Sequence
  Chemically synthetized sequence
gagactgctgtttatagctc

SEQ ID NO: 29 - DNA, 20 bp, Artificial Sequence
  Chemically synthetized sequence
actgggtgtatattctggta

SEQ ID NO: 30 - DNA, 20 bp, Artificial Sequence
  Chemically synthetized sequence
tgacgtattttgggcactct

SEQ ID NO: 31 - DNA, 20 bp, Artificial Sequence
  Chemically synthetized sequence
taaccatcctcaatttggca

SEQ ID NO: 32 - DNA, 20 bp, Artificial Sequence
  Chemically synthetized sequence
aatggacggagtgttcctta

SEQ ID NO: 33 - DNA, 20 bp, Artificial Sequence
  Chemically synthetized sequence
ctccaaatagacctctggat

SEQ ID NO: 34 - DNA, 20 bp, Artificial Sequence
  Chemically synthetized sequence
cccttcaataaaaccggcaa

SEQ ID NO: 35 - DNA, 20 bp, Artificial Sequence
  Chemically synthetized sequence
tttgtaatcccgttaatggc

SEQ ID NO: 36 - DNA, 20 bp, Artificial Sequence
  Chemically synthetized sequence
tctcgataacagtgttcacc

SEQ ID NO: 37 - DNA, 20 bp, Artificial Sequence
  Chemically synthetized sequence
gtccaaatgtccagaaatcc

SEQ ID NO: 38 - DNA, 20 bp, Artificial Sequence
  Chemically synthetized sequence
cttcacatttgagtcatgga

SEQ ID NO: 39 - DNA, 20 bp, Artificial Sequence
  Chemically synthetized sequence
ttggctttttactttctcat

SEQ ID NO: 40 - DNA, 20 bp, Artificial Sequence
  Chemically synthetized sequence
tccgatttctttggcattat

SEQ ID NO: 41 - DNA, 20 bp, Artificial Sequence
  Chemically synthetized sequence
gtcacacttgtggtagaact

SEQ ID NO: 42 - DNA, 20 bp, Artificial Sequence
  Chemically synthetized sequence
cttacactttccatgcattc

SEQ ID NO: 43 - DNA, 20 bp, Artificial Sequence
  Chemically synthetized sequence
gattccaatttcactccatc

SEQ ID NO: 44 - DNA, 20 bp, Artificial Sequence
  Chemically synthetized sequence
cagaatctgatagatcccca

SEQ ID NO: 45 - DNA, 20 bp, Artificial Sequence
  Chemically synthetized sequence
tggcgacagttgagtagatc

SEQ ID NO: 46 - DNA, 20 bp, Artificial Sequence
  Chemically synthetized sequence
gagaccaaaagcaccagtga

SEQ ID NO: 47 - DNA, 20 bp, Artificial Sequence
  Chemically synthetized sequence
acatccagaaactgattgcc

SEQ ID NO: 48 - DNA, 20 bp, Artificial Sequence
  Chemically synthetized sequence
atgcatattctgcactgcaa

SEQ ID NO: 49 - DNA, 20 bp, Artificial Sequence
  Chemically synthetized sequence
actaccagacagattgatcc

SEQ ID NO: 50 - DNA, 20 bp, Artificial Sequence
  Chemically synthetized sequence
ccctatttgcaatattaggc

SEQ ID NO: 51 - DNA, 20 bp, Artificial Sequence
  Chemically synthetized sequence
ggctaatccatattgagatt

SEQ ID NO: 52 - DNA, 20 bp, Artificial Sequence
  Chemically synthetized sequence
atggttttgacttccagttt

SEQ ID NO: 53 - DNA, 20 bp, Artificial Sequence
  Chemically synthetized sequence
atgttttggttgcatattcc

SEQ ID NO: 54 - DNA, 20 bp, Artificial Sequence
  Chemically synthetized sequence
cccaggtgctatttttatag

SEQ ID NO: 55 - DNA, 20 bp, Artificial Sequence
  Chemically synthetized sequence
tatcactgaagttgtgtcct

SEQ ID NO: 56 - DNA, 20 bp, Artificial Sequence
  Chemically synthetized sequence
ccacggatgggacaaagaga

SEQ ID NO: 57 - DNA, 20 bp, Artificial Sequence
  Chemically synthetized sequence
gctattgtctttgctgtata

SEQ ID NO: 58 - DNA, 20 bp, Artificial Sequence
  Chemically synthetized sequence
ctcctttggaaccaattctt

SEQ ID NO: 59 - DNA, 20 bp, Artificial Sequence
  Chemically synthetized sequence
aagggctctcttatgacaaa

SEQ ID NO: 60 - DNA, 20 bp, Artificial Sequence
  Chemically synthetized sequence
ggtcagaaaaaaggtcctgc

SEQ ID NO: 61 - DNA, 20 bp, Artificial Sequence
  Chemically synthetized sequence
cttgtcattcagtaaggcac

SEQ ID NO: 62 - DNA, 20 bp, Artificial Sequence
  Chemically synthetized sequence
ccttaacagtcccatttgaa

SEQ ID NO: 63 - DNA, 20 bp, Artificial Sequence
  Chemically synthetized sequence
ctcattaaggccctataagg

SEQ ID NO: 64 - DNA, 20 bp, Artificial Sequence
  Chemically synthetized sequence
acatgcacttgctgaccaag

SEQ ID NO: 65 - DNA, 20 bp, Artificial Sequence
  Chemically synthetized sequence
ggacctgaaattccgattgt

SEQ ID NO: 66 - DNA, 20 bp, Artificial Sequence
  Chemically synthetized sequence
tacagccactgctccattat

SEQ ID NO: 67 - DNA, 20 bp, Artificial Sequence
  Chemically synthetized sequence
ttattatgccgttgtatttt

SEQ ID NO: 68 - DNA, 20 bp, Artificial Sequence
  Chemically synthetized sequence
agactcttgtgtcctcaata

SEQ ID NO: 69 - DNA, 20 bp, Artificial Sequence
  Chemically synthetized sequence
gaaccatttacacaggcaca

SEQ ID NO: 70 - DNA, 20 bp, Artificial Sequence
  Chemically synthetized sequence
ggccatcagtcattatagta

SEQ ID NO: 71 - DNA, 20 bp, Artificial Sequence
  Chemically synthetized sequence
aaattttgtacgaggccagc

SEQ ID NO: 72 - DNA, 20 bp, Artificial Sequence
  Chemically synthetized sequence
tttagtaaccttcccctttt

SEQ ID NO: 73 - DNA, 20 bp, Artificial Sequence
  Chemically synthetized sequence
taggtgcattcaactctatt

SEQ ID NO: 74 - DNA, 20 bp, Artificial Sequence
  Chemically synthetized sequence
gaacattcctcatagtgaga

SEQ ID NO: 75 - DNA, 20 bp, Artificial Sequence
  Chemically synthetized sequence
tttgccggtatcagggtaac

SEQ ID NO: 76 - DNA, 20 bp, Artificial Sequence
  Chemically synthetized sequence
tgatcgaaagacacccatgg

SEQ ID NO: 77 - DNA, 20 bp, Artificial Sequence
  Chemically synthetized sequence
tcctatttgataatccaggt

SEQ ID NO: 78 - DNA, 20 bp, Artificial Sequence
  Chemically synthetized sequence
cgaaaaccccactgcagatg

SEQ ID NO: 79 - DNA, 20 bp, Artificial Sequence
  Chemically synthetized sequence
aaaatccctttactccgttt

SEQ ID NO: 80 - DNA, 20 bp, Artificial Sequence
  Chemically synthetized sequence
acaccattaccatacctata

SEQ ID NO: 81 - DNA, 20 bp, Artificial Sequence
  Chemically synthetized sequence
gacttttggtccttcctatc

SEQ ID NO: 82 - DNA, 20 bp, Artificial Sequence
  Chemically synthetized sequence
tcaaacccatgtctggaact

SEQ ID NO: 83 - DNA, 20 bp, Artificial Sequence
  Chemically synthetized sequence
tccattaggatcccaaatca

SEQ ID NO: 84 - DNA, 20 bp, Artificial Sequence
  Chemically synthetized sequence
acttactatcagtctctgtc

SEQ ID NO: 85 - DNA, 20 bp, Artificial Sequence
  Chemically synthetized sequence
ccacaacatcttgcctaaca

SEQ ID NO: 86 - DNA, 20 bp, Artificial Sequence
  Chemically synthetized sequence
tataccctgaccaatcagtc

SEQ ID NO: 87 - DNA, 20 bp, Artificial Sequence
  Chemically synthetized sequence
ctgttagctcaggatgttga

SEQ ID NO: 88 - DNA, 20 bp, Artificial Sequence
  Chemically synthetized sequence
cacggcctcatacagtctag

SEQ ID NO: 89 - DNA, 20 bp, Artificial Sequence
  Chemically synthetized sequence
gccaagaccaatctacagta

SEQ ID NO: 90 - DNA, 20 bp, Artificial Sequence
  Chemically synthetized sequence
aatggcaactcagcaccgtc

SEQ ID NO: 91 - DNA, 20 bp, Artificial Sequence
  Chemically synthetized sequence
aacagactacttgtcaatgc

SEQ ID NO: 92 - DNA, 20 bp, Artificial Sequence
  Chemically synthetized sequence
cactcagtgagtgattatct

SEQ ID NO: 93 - DNA, 20 bp, Artificial Sequence
  Chemically synthetized sequence
tgggacgccatgattttgat

SEQ ID NO: 94 - DNA, 20 bp, Artificial Sequence
  Chemically synthetized sequence
atctgttcgtaagaccgttt

SEQ ID NO: 95 - DNA, 20 bp, Artificial Sequence
  Chemically synthetized sequence
tgatttcagtggcattctgg

SEQ ID NO: 96 - DNA, 20 bp, Artificial Sequence
  Chemically synthetized sequence
atcatttttccgacggatgc

SEQ ID NO: 97 - DNA, 20 bp, Artificial Sequence
  Chemically synthetized sequence
gtagaatcgtccaattccac

SEQ ID NO: 98 - DNA, 20 bp, Artificial Sequence
  Chemically synthetized sequence
ttgagttcggtgcacatttg

SEQ ID NO: 99 - DNA, 20 bp, Artificial Sequence
  Chemically synthetized sequence
atcaaccgtccctcataatc

SEQ ID NO: 100 - DNA, 20 bp, Artificial Sequence
  Chemically synthetized sequence
ctctctattgttaagctgtt

SEQ ID NO: 101 - DNA, 20 bp, Artificial Sequence
  Chemically synthetized sequence
gtcaaaagcagagagcacca

SEQ ID NO: 102 - DNA, 20 bp, Artificial Sequence
  Chemically synthetized sequence
ccagttttcttaggatcttt

SEQ ID NO: 103 - DNA, 20 bp, Artificial Sequence
  Chemically synthetized sequence
tactctcctgtatataggtc

SEQ ID NO: 104 - DNA, 20 bp, Artificial Sequence
  Chemically synthetized sequence
tcgtcaccattattagcttg

SEQ ID NO: 105 - DNA, 20 bp, Artificial Sequence
  Chemically synthetized sequence
atgccagatcatcatgtgag

SEQ ID NO: 106 - DNA, 20 bp, Artificial Sequence
  Chemically synthetized sequence
tcctctgataagttgcatca

SEQ ID NO: 107 - DNA, 20 bp, Artificial Sequence
  Chemically synthetized sequence
cattccggtgcgaacaagag

SEQ ID NO: 108 - DNA, 20 bp, Artificial Sequence
  Chemically synthetized sequence
ttgcatcagagagcacatcc

SEQ ID NO: 109 - DNA, 20 bp, Artificial Sequence
  Chemically synthetized sequence
acctcctagggagagttgaa

SEQ ID NO: 110 - DNA, 20 bp, Artificial Sequence
  Chemically synthetized sequence
gtttgatcatcctgaccaat

SEQ ID NO: 111 - DNA, 20 bp, Artificial Sequence
  Chemically synthetized sequence
gaagttccgatcattgatcc

SEQ ID NO: 112 - DNA, 20 bp, Artificial Sequence
  Chemically synthetized sequence
ttcttgtttttcgtccattc

SEQ ID NO: 113 - DNA, 20 bp, Artificial Sequence
  Chemically synthetized sequence
tttccctttgagaatgttgc

SEQ ID NO: 114 - DNA, 20 bp, Artificial Sequence
  Chemically synthetized sequence
ctttttgtgcagcagtttga

SEQ ID NO: 115 - DNA, 20 bp, Artificial Sequence
  Chemically synthetized sequence
tctctcacttgatccatcat

SEQ ID NO: 116 - DNA, 20 bp, Artificial Sequence
  Chemically synthetized sequence
gatcttcgaactcagcattc

SEQ ID NO: 117 - DNA, 20 bp, Artificial Sequence
  Chemically synthetized sequence
gcagaccgtgctagaaaagt

SEQ ID NO: 118 - DNA, 20 bp, Artificial Sequence
  Chemically synthetized sequence
aaccgaccctctcaatatga

SEQ ID NO: 119 - DNA, 20 bp, Artificial Sequence
  Chemically synthetized sequence
cctctctttcaaagtcgtac

SEQ ID NO: 120 - DNA, 20 bp, Artificial Sequence
  Chemically synthetized sequence
tctgaaagggtctattccga

SEQ ID NO: 121 - DNA, 20 bp, Artificial Sequence
  Chemically synthetized sequence
tgtacacttggctgttttga

SEQ ID NO: 122 - DNA, 20 bp, Artificial Sequence
  Chemically synthetized sequence
tggattctcatttggtctga

SEQ ID NO: 123 - DNA, 20 bp, Artificial Sequence
  Chemically synthetized sequence
cacaccagttgactcttgtg

SEQ ID NO: 124 - DNA, 20 bp, Artificial Sequence
  Chemically synthetized sequence
ttcaaatgcggcagaatggc

SEQ ID NO: 125 - DNA, 20 bp, Artificial Sequence
  Chemically synthetized sequence
gtccctttgatgaagcttaa

SEQ ID NO: 126 - DNA, 20 bp, Artificial Sequence
  Chemically synthetized sequence
agtggaaagcttccctcttg

SEQ ID NO: 127 - DNA, 20 bp, Artificial Sequence
  Chemically synthetized sequence
tggaagcaatttgaactcct

SEQ ID NO: 128 - DNA, 20 bp, Artificial Sequence
  Chemically synthetized sequence
gattccatagtctccatatt

SEQ ID NO: 129 - DNA, 20 bp, Artificial Sequence
  Chemically synthetized sequence
tgcttctcagttcaagtgta

SEQ ID NO: 130 - DNA, 20 bp, Artificial Sequence
  Chemically synthetized sequence
tctgttgattggtgtttcct

SEQ ID NO: 131 - DNA, 20 bp, Artificial Sequence
  Chemically synthetized sequence
acgtaggttgtatgctgatt

SEQ ID NO: 132 - DNA, 20 bp, Artificial Sequence
  Chemically synthetized sequence
gggagatttctctgtactga

SEQ ID NO: 133 - DNA, 20 bp, Artificial Sequence
  Chemically synthetized sequence
ccataatggttgttctgtca

SEQ ID NO: 134 - DNA, 20 bp, Artificial Sequence
  Chemically synthetized sequence
tctgtattcccattgaatgc

SEQ ID NO: 135 - DNA, 20 bp, Artificial Sequence
  Chemically synthetized sequence
cctcatgtcagatgttcttc

SEQ ID NO: 136 - DNA, 20 bp, Artificial Sequence
  Chemically synthetized sequence
ctttccatcatccttatgat

SEQ ID NO: 137 - DNA, 20 bp, Artificial Sequence
  Chemically synthetized sequence
agacacatcttctggtcttg

SEQ ID NO: 138 - DNA, 20 bp, Artificial Sequence
  Chemically synthetized sequence
tttcgtccgagagctcgaag

SEQ ID NO: 139 - DNA, 20 bp, Artificial Sequence
  Chemically synthetized sequence
caaaggaaggcacgatcggg

SEQ ID NO: 140 - DNA, 20 bp, Artificial Sequence
  Chemically synthetized sequence
atcttccattttggatcagt

SEQ ID NO: 141 - DNA, 20 bp, Artificial Sequence
  Chemically synthetized sequence
gattgaagcattgtcgcaca

SEQ ID NO: 142 - DNA, 20 bp, Artificial Sequence
  Chemically synthetized sequence
aagtgagtgcatattgctgc

SEQ ID NO: 143 - DNA, 20 bp, Artificial Sequence
  Chemically synthetized sequence
tacgattattgactcgcctt

SEQ ID NO: 144 - DNA, 20 bp, Artificial Sequence
  Chemically synthetized sequence
tcttccctcgattatttcaa

SEQ ID NO: 145 - DNA, 20 bp, Artificial Sequence
  Chemically synthetized sequence
actgtttactactgtccagg

SEQ ID NO: 146 - DNA, 20 bp, Artificial Sequence
  Chemically synthetized sequence
gaaactttggtttctcagcc

SEQ ID NO: 147 - DNA, 20 bp, Artificial Sequence
  Chemically synthetized sequence
aacttctctccttgttactc

SEQ ID NO: 148 - DNA, 20 bp, Artificial Sequence
  Chemically synthetized sequence
gccttttccagatagtatat

SEQ ID NO: 149 - DNA, 20 bp, Artificial Sequence
  Chemically synthetized sequence
ttccccagtgaacgagaaaa

SEQ ID NO: 150 - DNA, 20 bp, Artificial Sequence
  Chemically synthetized sequence
tgctttcttcatcgagagtg

SEQ ID NO: 151 - DNA, 20 bp, Artificial Sequence
  Chemically synthetized sequence
tagtctggttttgatcctag

SEQ ID NO: 152 - DNA, 20 bp, Artificial Sequence
  Chemically synthetized sequence
ccatttcttgtcttatggtg

SEQ ID NO: 153 - DNA, 20 bp, Artificial Sequence
  Chemically synthetized sequence
tgttcctgtgatttcaaacc

SEQ ID NO: 154 - DNA, 20 bp, Artificial Sequence
  Chemically synthetized sequence
ttcaaggctggagaagttcg

SEQ ID NO: 155 - DNA, 20 bp, Artificial Sequence
  Chemically synthetized sequence
gttcgaatccatccacatag

SEQ ID NO: 156 - DNA, 20 bp, Artificial Sequence
  Chemically synthetized sequence
ggacatttgagacagcttgc

SEQ ID NO: 157 - DNA, 20 bp, Artificial Sequence
  Chemically synthetized sequence
ccattcggaagtctaagtgg

SEQ ID NO: 158 - DNA, 20 bp, Artificial Sequence
  Chemically synthetized sequence
atttggaccgctgagaacag

SEQ ID NO: 159 - DNA, 20 bp, Artificial Sequence
  Chemically synthetized sequence
attttaaggcatccatcagc

SEQ ID NO: 160 - DNA, 20 bp, Artificial Sequence
  Chemically synthetized sequence
gttctcatgcatttgattgc

SEQ ID NO: 161 - DNA, 20 bp, Artificial Sequence
  Chemically synthetized sequence
ttggatttattcccttttcg

SEQ ID NO: 162 - DNA, 20 bp, Artificial Sequence
  Chemically synthetized sequence
cttgcttccatgacagaaga

SEQ ID NO: 163 - DNA, 20 bp, Artificial Sequence
  Chemically synthetized sequence
ggaattttctcctcattctc

SEQ ID NO: 164 - DNA, 20 bp, Artificial Sequence
  Chemically synthetized sequence
gtcgtcaaagtctacctttt

SEQ ID NO: 165 - DNA, 20 bp, Artificial Sequence
  Chemically synthetized sequence
gcttcaaatcacctacatct

SEQ ID NO: 166 - DNA, 20 bp, Artificial Sequence
  Chemically synthetized sequence
ctcaattctggttcatcact

SEQ ID NO: 167 - DNA, 20 bp, Artificial Sequence
  Chemically synthetized sequence
tctgaatccaacttgctagc

SEQ ID NO: 168 - DNA, 20 bp, Artificial Sequence
  Chemically synthetized sequence
ttcgcatgccttgttaaact

SEQ ID NO: 169 - DNA, 20 bp, Artificial Sequence
  Chemically synthetized sequence
ctctatccagcttgaatctg

SEQ ID NO: 170 - DNA, 20 bp, Artificial Sequence
  Chemically synthetized sequence
cttctcatgcttgcaatgtg

SEQ ID NO: 171 - DNA, 20 bp, Artificial Sequence
  Chemically synthetized sequence
aggcagtattgatgtacacc

SEQ ID NO: 172 - DNA, 20 bp, Artificial Sequence
  Chemically synthetized sequence
cattgctgcacaagatgcat

SEQ ID NO: 173 - DNA, 20 bp, Artificial Sequence
  Chemically synthetized sequence
ccatacaagttggtctttcg

SEQ ID NO: 174 - DNA, 20 bp, Artificial Sequence
  Chemically synthetized sequence
tgtcattccttaagtgggat

SEQ ID NO: 175 - DNA, 20 bp, Artificial Sequence
  Chemically synthetized sequence
gcctatggcacttcttataa

SEQ ID NO: 176 - DNA, 20 bp, Artificial Sequence
  Chemically synthetized sequence
acatacaagaacatgggcct

SEQ ID NO: 177 - DNA, 20 bp, Artificial Sequence
  Chemically synthetized sequence
aagtgactggaggaggcaac

SEQ ID NO: 178 - DNA, 20 bp, Artificial Sequence
  Chemically synthetized sequence
agaggactcagcttcaatca

SEQ ID NO: 179 - DNA, 20 bp, Artificial Sequence
  Chemically synthetized sequence
tggtcatgtctttctctttg

SEQ ID NO: 180 - DNA, 20 bp, Artificial Sequence
  Chemically synthetized sequence
ccatgtttctgatttgttct

SEQ ID NO: 181 - DNA, 20 bp, Artificial Sequence
  Chemically synthetized sequence
ataaagtcctgcagaccttc

SEQ ID NO: 182 - DNA, 20 bp, Artificial Sequence
  Chemically synthetized sequence
ctgttgaataccgactttgc

SEQ ID NO: 183 - DNA, 20 bp, Artificial Sequence
  Chemically synthetized sequence
atccttctagttgtggagat

SEQ ID NO: 184 - DNA, 20 bp, Artificial Sequence
  Chemically synthetized sequence
agcagttttcttgattcagc

SEQ ID NO: 185 - DNA, 20 bp, Artificial Sequence
  Chemically synthetized sequence
aatcaggcactcctcaattg

SEQ ID NO: 186 - DNA, 20 bp, Artificial Sequence
  Chemically synthetized sequence
gcattaagcaaaacccaggg

SEQ ID NO: 187 - DNA, 20 bp, Artificial Sequence
  Chemically synthetized sequence
caaatagtagcactgccaca

SEQ ID NO: 188 - DNA, 20 bp, Artificial Sequence
  Chemically synthetized sequence
taaggtcggattgacatcca

SEQ ID NO: 189 - DNA, 20 bp, Artificial Sequence
  Chemically synthetized sequence
attttgtgctggcactttta

SEQ ID NO: 190 - DNA, 20 bp, Artificial Sequence
  Chemically synthetized sequence
aaggagggtctccagtataa

SEQ ID NO: 191 - DNA, 20 bp, Artificial Sequence
  Chemically synthetized sequence
tactgatgtgtcctgttgac

SEQ ID NO: 192 - DNA, 20 bp, Artificial Sequence
  Chemically synthetized sequence
catcaatcgggttgagttgc

SEQ ID NO: 193 - DNA, 20 bp, Artificial Sequence
  Chemically synthetized sequence
acttggttcattgtcttctg

SEQ ID NO: 194 - DNA, 20 bp, Artificial Sequence
  Chemically synthetized sequence
ctactcgtgtttgctgaaca

SEQ ID NO: 195 - DNA, 20 bp, Artificial Sequence
  Chemically synthetized sequence
tttagagtccagtcataggt

SEQ ID NO: 196 - DNA, 20 bp, Artificial Sequence
  Chemically synthetized sequence
ttctattgtgttggccaatg

SEQ ID NO: 197 - DNA, 20 bp, Artificial Sequence
  Chemically synthetized sequence
tgaggccatttgatctgaac

SEQ ID NO: 198 - DNA, 20 bp, Artificial Sequence
  Chemically synthetized sequence
aaggaagtctatgagccttc

SEQ ID NO: 199 - DNA, 20 bp, Artificial Sequence
  Chemically synthetized sequence
tcattgactccattacatcc

SEQ ID NO: 200 - DNA, 20 bp, Artificial Sequence
  Chemically synthetized sequence
tcattgtgttcagggtcaat

SEQ ID NO: 201 - DNA, 20 bp, Artificial Sequence
  Chemically synthetized sequence
gccagtgtctcaacaaagta

SEQ ID NO: 202 - DNA, 20 bp, Artificial Sequence
  Chemically synthetized sequence
caaccctgattgttcaagtt

SEQ ID NO: 203 - DNA, 20 bp, Artificial Sequence
  Chemically synthetized sequence
ggtcatcatcttccttacaa

SEQ ID NO: 204 - DNA, 20 bp, Artificial Sequence
  Chemically synthetized sequence
agaaagttcggtgtcctgag

SEQ ID NO: 205 - DNA, 20 bp, Artificial Sequence
  Chemically synthetized sequence
atttggtgttatctccagtg

SEQ ID NO: 206 - DNA, 20 bp, Artificial Sequence
  Chemically synthetized sequence
tccgaggattctgattttcg

SEQ ID NO: 207 - DNA, 20 bp, Artificial Sequence
  Chemically synthetized sequence
catatatgtgatcatggcca

SEQ ID NO: 208 - DNA, 20 bp, Artificial Sequence
  Chemically synthetized sequence
acatttctgaaccattcggg

SEQ ID NO: 209 - DNA, 20 bp, Artificial Sequence
  Chemically synthetized sequence
ctcgccattttgtttgagaa

SEQ ID NO: 210 - DNA, 20 bp, Artificial Sequence
  Chemically synthetized sequence
tgctctcaaacatataccct

SEQ ID NO: 211 - DNA, 20 bp, Artificial Sequence
  Chemically synthetized sequence
agcatttctgcaggtatttg

SEQ ID NO: 212 - DNA, 20 bp, Artificial Sequence
  Chemically synthetized sequence
agcggtcggattttttcaat

SEQ ID NO: 213 - DNA, 20 bp, Artificial Sequence
  Chemically synthetized sequence
catattgaacatgcccatca

SEQ ID NO: 214 - DNA, 20 bp, Artificial Sequence
  Chemically synthetized sequence
tggagacgcctaatacagtg

SEQ ID NO: 215 - DNA, 20 bp, Artificial Sequence
  Chemically synthetized sequence
tctcttttgtccaagattca

SEQ ID NO: 216 - DNA, 20 bp, Artificial Sequence
  Chemically synthetized sequence
accagtaagtagtcttggtg

SEQ ID NO: 217 - DNA, 20 bp, Artificial Sequence
  Chemically synthetized sequence
cgtcagaggattgaagacca

SEQ ID NO: 218 - DNA, 20 bp, Artificial Sequence
  Chemically synthetized sequence
gggtgcattcacaatcagag

SEQ ID NO: 219 - DNA, 20 bp, Artificial Sequence
  Chemically synthetized sequence
ataaaacctgtcgactccgg

SEQ ID NO: 220 - DNA, 20 bp, Artificial Sequence
  Chemically synthetized sequence
ctccatgctgaaattggcaa

SEQ ID NO: 221 - DNA, 20 bp, Artificial Sequence
  Chemically synthetized sequence
aactccaatactcatgtccg

SEQ ID NO: 222 - DNA, 20 bp, Artificial Sequence
  Chemically synthetized sequence
ttgctggaccaagatcattg

SEQ ID NO: 223 - DNA, 20 bp, Artificial Sequence
  Chemically synthetized sequence
acaactgaagggccatttga

SEQ ID NO: 224 - DNA, 20 bp, Artificial Sequence
  Chemically synthetized sequence
gtacgtgtacctgtaatctt

SEQ ID NO: 225 - DNA, 20 bp, Artificial Sequence
  Chemically synthetized sequence
ttgtataaatttgggcctcc

SEQ ID NO: 226 - DNA, 20 bp, Artificial Sequence
  Chemically synthetized sequence
aggcagacttcaggaatgtg

SEQ ID NO: 227 - DNA, 20 bp, Artificial Sequence
  Chemically synthetized sequence
ggtaatcctcatccatcaat

SEQ ID NO: 228 - DNA, 20 bp, Artificial Sequence
  Chemically synthetized sequence
caaatgggttcagtgggttg

SEQ ID NO: 229 - DNA, 20 bp, Artificial Sequence
  Chemically synthetized sequence
agcatcatactccatgtttt

SEQ ID NO: 230 - DNA, 20 bp, Artificial Sequence
  Chemically synthetized sequence
tcaagtactcctctttgact

SEQ ID NO: 231 - DNA, 20 bp, Artificial Sequence
  Chemically synthetized sequence
acctttggtacatttgttca

SEQ ID NO: 232 - DNA, 20 bp, Artificial Sequence
  Chemically synthetized sequence
tggtcttctgtatgaactgc

SEQ ID NO: 233 - DNA, 20 bp, Artificial Sequence
  Chemically synthetized sequence
tcaatccgtgcatcaattcg

SEQ ID NO: 234 - DNA, 20 bp, Artificial Sequence
  Chemically synthetized sequence
gctcttcaatggtggaacag

SEQ ID NO: 235 - DNA, 20 bp, Artificial Sequence
  Chemically synthetized sequence
attcactatttttgccgtct

SEQ ID NO: 236 - DNA, 20 bp, Artificial Sequence
  Chemically synthetized sequence
gcgagactgcgacattagat

SEQ ID NO: 237 - DNA, 20 bp, Artificial Sequence
  Chemically synthetized sequence
cggtggtttttgtgagtatc

SEQ ID NO: 238 - DNA, 20 bp, Artificial Sequence
  Chemically synthetized sequence
ctcttgtctgctgtaattgg

SEQ ID NO: 239 - DNA, 20 bp, Artificial Sequence
  Chemically synthetized sequence
ttgctcatttctctcaggaa

SEQ ID NO: 240 - DNA, 20 bp, Artificial Sequence
  Chemically synthetized sequence
gatcctgcatcattcatttt

SEQ ID NO: 241 - DNA, 20 bp, Artificial Sequence
  Chemically synthetized sequence
aaatggacagggccaaaggt

SEQ ID NO: 242 - DNA, 20 bp, Artificial Sequence
  Chemically synthetized sequence
tccgacgtattttgacttgg

SEQ ID NO: 243 - DNA, 20 bp, Artificial Sequence
  Chemically synthetized sequence
tctgcatgaccaggatttat

SEQ ID NO: 244 - DNA, 20 bp, Artificial Sequence
  Chemically synthetized sequence
ttccatgattacatcctgtg

SEQ ID NO: 245 - DNA, 20 bp, Artificial Sequence
  Chemically synthetized sequence
cccacttcgttagggaaaac

SEQ ID NO: 246 - DNA, 20 bp, Artificial Sequence
  Chemically synthetized sequence
ttatcgttagttgcgattcc

SEQ ID NO: 247 - DNA, 20 bp, Artificial Sequence
  Chemically synthetized sequence
ccaacatgtatgcaaccatc

SEQ ID NO: 248 - DNA, 20 bp, Artificial Sequence
  Chemically synthetized sequence
ctgggaggaatctcgttttg

SEQ ID NO: 249 - DNA, 20 bp, Artificial Sequence
  Chemically synthetized sequence
tgttccttgagtcaaatgca

SEQ ID NO: 250 - DNA, 20 bp, Artificial Sequence
  Chemically synthetized sequence
ctggagtatacatctgttcc

SEQ ID NO: 251 - DNA, 20 bp, Artificial Sequence
  Chemically synthetized sequence
ctagtggatctgctgatact

SEQ ID NO: 252 - DNA, 20 bp, Artificial Sequence
  Chemically synthetized sequence
ctaattccaccaatctgtgt

SEQ ID NO: 253 - DNA, 20 bp, Artificial Sequence
  Chemically synthetized sequence
ttctgcctaaggatgtctac

SEQ ID NO: 254 - DNA, 20 bp, Artificial Sequence
  Chemically synthetized sequence
gatgagctaattctcagtcc

SEQ ID NO: 255 - DNA, 20 bp, Artificial Sequence
  Chemically synthetized sequence
gatccgcttgttctcttaaa

SEQ ID NO: 256 - DNA, 20 bp, Artificial Sequence
  Chemically synthetized sequence
gtttgaagattgcccgtaag

SEQ ID NO: 257 - DNA, 20 bp, Artificial Sequence
  Chemically synthetized sequence
ctcatgcactcttatcttca

SEQ ID NO: 258 - DNA, 20 bp, Artificial Sequence
  Chemically synthetized sequence
ccaaccattgtgaactcttc

SEQ ID NO: 259 - DNA, 20 bp, Artificial Sequence
  Chemically synthetized sequence
atcagctgaatcaatctcct

SEQ ID NO: 260 - DNA, 20 bp, Artificial Sequence
  Chemically synthetized sequence
atggccacaattattgcttc

SEQ ID NO: 261 - DNA, 20 bp, Artificial Sequence
  Chemically synthetized sequence
attcagatcacctctgactg

SEQ ID NO: 262 - DNA, 20 bp, Artificial Sequence
  Chemically synthetized sequence
gcataggattcaatcgctga

SEQ ID NO: 263 - DNA, 20 bp, Artificial Sequence
  Chemically synthetized sequence
actttcgcatccttctgaaa

SEQ ID NO: 264 - DNA, 20 bp, Artificial Sequence
  Chemically synthetized sequence
aatcattcccatcacattgt

SEQ ID NO: 265 - DNA, 20 bp, Artificial Sequence
  Chemically synthetized sequence
ggagtcatgtctggcaatat

SEQ ID NO: 266 - DNA, 20 bp, Artificial Sequence
  Chemically synthetized sequence
tctcattgacatctcgatgc

SEQ ID NO: 267 - DNA, 20 bp, Artificial Sequence
  Chemically synthetized sequence
tactcatctacacccatttt

SEQ ID NO: 268 - DNA, 20 bp, Artificial Sequence
  Chemically synthetized sequence
tggtcccggattctcaaaaa

SEQ ID NO: 269 - DNA, 20 bp, Artificial Sequence
  Chemically synthetized sequence
ctcgggagacagtagtacat

SEQ ID NO: 270 - DNA, 20 bp, Artificial Sequence
  Chemically synthetized sequence
ccacatcattgacgatgagt

SEQ ID NO: 271 - DNA, 20 bp, Artificial Sequence
  Chemically synthetized sequence
ccattgataggtattgacca

SEQ ID NO: 272 - DNA, 20 bp, Artificial Sequence
  Chemically synthetized sequence
tctaatggccttaggtacta

SEQ ID NO: 273 - DNA, 20 bp, Artificial Sequence
  Chemically synthetized sequence
agttcttacaaacccactgt

SEQ ID NO: 274 - DNA, 20 bp, Artificial Sequence
  Chemically synthetized sequence
tcaaatgtcccaagcacatc

SEQ ID NO: 275 - DNA, 20 bp, Artificial Sequence
  Chemically synthetized sequence
gctgcgaagggaagaagttt

SEQ ID NO: 276 - DNA, 20 bp, Artificial Sequence
  Chemically synthetized sequence
actgcattctactttgcttt

SEQ ID NO: 277 - DNA, 20 bp, Artificial Sequence
  Chemically synthetized sequence
gtggccttgttatagttgaa

SEQ ID NO: 278 - DNA, 20 bp, Artificial Sequence
  Chemically synthetized sequence
aatgaggaatcccctcagaa

SEQ ID NO: 279 - DNA, 20 bp, Artificial Sequence
  Chemically synthetized sequence
ccatatctcttgtcttcttt

SEQ ID NO: 280 - DNA, 20 bp, Artificial Sequence
  Chemically synthetized sequence
caaggttgctcagttcattg

SEQ ID NO: 281 - DNA, 20 bp, Artificial Sequence
  Chemically synthetized sequence
ttgcccaattagcacattag

SEQ ID NO: 282 - DNA, 20 bp, Artificial Sequence
  Chemically synthetized sequence
ttccgtttcattaccaacac

SEQ ID NO: 283 - DNA, 20 bp, Artificial Sequence
  Chemically synthetized sequence
aattgatggccatccgaatt

SEQ ID NO: 284 - DNA, 20 bp, Artificial Sequence
  Chemically synthetized sequence
tacgtttcgacctcggttag

SEQ ID NO: 285 - DNA, 20 bp, Artificial Sequence
  Chemically synthetized sequence
cctgacgggatgatagagag

SEQ ID NO: 286 - DNA, 20 bp, Artificial Sequence
  Chemically synthetized sequence
cctgcaaagacatcttcaag

SEQ ID NO: 287 - DNA, 20 bp, Artificial Sequence
  Chemically synthetized sequence
aacctcaagatcagtgttct

SEQ ID NO: 288 - DNA, 20 bp, Artificial Sequence
  Chemically synthetized sequence
ttgtctttagccattccatg

SEQ ID NO: 289 - DNA, 20 bp, Artificial Sequence
  Chemically synthetized sequence
gtcagaggtgacaggattgg

SEQ ID NO: 290 - DNA, 20 bp, Artificial Sequence
  Chemically synthetized sequence
cacaaatcctaaaatcccct

SEQ ID NO: 291 - DNA, 20 bp, Artificial Sequence
  Chemically synthetized sequence
gcttcctatacagtttaact

SEQ ID NO: 292 - DNA, 20 bp, Artificial Sequence
  Chemically synthetized sequence
ctgagtgagatttctttggc

SEQ ID NO: 293 - DNA, 20 bp, Artificial Sequence
  Chemically synthetized sequence
catacaactggcaagtgcac

SEQ ID NO: 294 - DNA, 20 bp, Artificial Sequence
  Chemically synthetized sequence
ccatcctgttgtatatgagg

SEQ ID NO: 295 - DNA, 20 bp, Artificial Sequence
  Chemically synthetized sequence
caaatgccacttcagtggtc

SEQ ID NO: 296 - DNA, 20 bp, Artificial Sequence
  Chemically synthetized sequence
tcacaggttgcacataccag

SEQ ID NO: 297 - DNA, 20 bp, Artificial Sequence
  Chemically synthetized sequence
atgctgggagtcagcaatct

SEQ ID NO: 298 - DNA, 20 bp, Artificial Sequence
  Chemically synthetized sequence
tcaccatttgcctatgagac

SEQ ID NO: 299 - DNA, 20 bp, Artificial Sequence
  Chemically synthetized sequence
ctgattagtggattggttgt

SEQ ID NO: 300 - DNA, 20 bp, Artificial Sequence
  Chemically synthetized sequence
agctgtagtgctggctaaaa

SEQ ID NO: 301 - DNA, 20 bp, Artificial Sequence
  Chemically synthetized sequence
tcactcgatccagccatttg

SEQ ID NO: 302 - DNA, 20 bp, Artificial Sequence
  Chemically synthetized sequence
ctgactagcaacctccatgg

SEQ ID NO: 303 - DNA, 20 bp, Artificial Sequence
  Chemically synthetized sequence
ctcatcgcttgcaccatttg

SEQ ID NO: 304 - DNA, 20 bp, Artificial Sequence
  Chemically synthetized sequence
agctaggatgagtcccaatg

SEQ ID NO: 305 - DNA, 20 bp, Artificial Sequence
  Chemically synthetized sequence
gatcatttttcagaccagca

SEQ ID NO: 306 - DNA, 20 bp, Artificial Sequence
  Chemically synthetized sequence
tttctgataggcctgcaaat

SEQ ID NO: 307 - DNA, 20 bp, Artificial Sequence
  Chemically synthetized sequence
gatcccaatgatatttgcgg

SEQ ID NO: 308 - DNA, 20 bp, Artificial Sequence
  Chemically synthetized sequence
gaatccacaatgtcaagtgc

SEQ ID NO: 309 - DNA, 20 bp, Artificial Sequence
  Chemically synthetized sequence
tcctttcagtccgtatttaa

SEQ ID NO: 310 - DNA, 20 bp, Artificial Sequence
  Chemically synthetized sequence
atagactttggcactccttc

SEQ ID NO: 311 - DNA, 20 bp, Artificial Sequence
  Chemically synthetized sequence
ctgttcctttcgatattctt

SEQ ID NO: 312 - DNA, 20 bp, Artificial Sequence
  Chemically synthetized sequence
aaatgaccatcgtcagcatc

SEQ ID NO: 313 - DNA, 20 bp, Artificial Sequence
  Chemically synthetized sequence
gttttttactccagctctat

SEQ ID NO: 314 - DNA, 20 bp, Artificial Sequence
  Chemically synthetized sequence
ggatccattatgtctttgtc

SEQ ID NO: 315 - DNA, 20 bp, Artificial Sequence
  Chemically synthetized sequence
ctgaaagcttgacacagtgt

SEQ ID NO: 316 - DNA, 20 bp, Artificial Sequence
  Chemically synthetized sequence
tagttcttggtctgcaactc

SEQ ID NO: 317 - DNA, 20 bp, Artificial Sequence
  Chemically synthetized sequence
gatcaaggaatggggcatca

SEQ ID NO: 318 - DNA, 20 bp, Artificial Sequence
  Chemically synthetized sequence
gatttctgatctcggcgaag

SEQ ID NO: 319 - DNA, 20 bp, Artificial Sequence
  Chemically synthetized sequence
cactatctgctttccagcac

SEQ ID NO: 320 - DNA, 20 bp, Artificial Sequence
  Chemically synthetized sequence
attcttctttcagaatccgc

SEQ ID NO: 321 - DNA, 20 bp, Artificial Sequence
  Chemically synthetized sequence
gtcattttaagtgcctcatc

SEQ ID NO: 322 - DNA, 20 bp, Artificial Sequence
  Chemically synthetized sequence
tgtcagttaggtaacgcgac

SEQ ID NO: 323 - DNA, 20 bp, Artificial Sequence
  Chemically synthetized sequence
ccttgacatttcctcaagag

SEQ ID NO: 324 - DNA, 20 bp, Artificial Sequence
  Chemically synthetized sequence
attctgatacaaagagggcc

SEQ ID NO: 325 - DNA, 20 bp, Artificial Sequence
  Chemically synthetized sequence
gccggtcaaaaatcacactg

SEQ ID NO: 326 - DNA, 20 bp, Artificial Sequence
  Chemically synthetized sequence
gcccttagcaatattagagt

SEQ ID NO: 327 - DNA, 20 bp, Artificial Sequence
  Chemically synthetized sequence
aatttcgccaacaattgctc

SEQ ID NO: 328 - DNA, 20 bp, Artificial Sequence
  Chemically synthetized sequence
ctggaagagaaggcaatggt

SEQ ID NO: 329 - DNA, 20 bp, Artificial Sequence
  Chemically synthetized sequence
ttgacatcctcagcagtatg

SEQ ID NO: 330 - DNA, 20 bp, Artificial Sequence
  Chemically synthetized sequence
gatgaggactccaactgcat

SEQ ID NO: 331 - DNA, 20 bp, Artificial Sequence
  Chemically synthetized sequence
ttcagagactcgaactgtgt

SEQ ID NO: 332 - DNA, 20 bp, Artificial Sequence
  Chemically synthetized sequence
tccaagcgaatctctgtaga

SEQ ID NO: 333 - DNA, 20 bp, Artificial Sequence
  Chemically synthetized sequence
gtctcccattctcattactg

SEQ ID NO: 334 - DNA, 20 bp, Artificial Sequence
  Chemically synthetized sequence
gtttctgttttggagtgagt

SEQ ID NO: 335 - DNA, 20 bp, Artificial Sequence
  Chemically synthetized sequence
gcaatagatgtaaggcttgc

SEQ ID NO: 336 - DNA, 20 bp, Artificial Sequence
  Chemically synthetized sequence
aagttcttatctcttgctcc

SEQ ID NO: 337 - DNA, 20 bp, Artificial Sequence
  Chemically synthetized sequence
cccctgatgtgagaaatagg

SEQ ID NO: 338 - DNA, 20 bp, Artificial Sequence
  Chemically synthetized sequence
ggtgcagttatttctgtttg

SEQ ID NO: 339 - DNA, 20 bp, Artificial Sequence
  Chemically synthetized sequence
ttgcacaatccaatggcatt

SEQ ID NO: 340 - DNA, 20 bp, Artificial Sequence
  Chemically synthetized sequence
gttcacagcctgaacatttg

SEQ ID NO: 341 - DNA, 20 bp, Artificial Sequence
  Chemically synthetized sequence
ggttctgggagaagaagtgt

SEQ ID NO: 342 - DNA, 20 bp, Artificial Sequence
  Chemically synthetized sequence
cgggcaagataaacgagggt

SEQ ID NO: 343 - DNA, 20 bp, Artificial Sequence
  Chemically synthetized sequence
ggctaattaggagtgctttc

SEQ ID NO: 344 - DNA, 20 bp, Artificial Sequence
  Chemically synthetized sequence
ctttggtttctccgaatcta

SEQ ID NO: 345 - DNA, 20 bp, Artificial Sequence
  Chemically synthetized sequence
aggttcccttattatcaagg

SEQ ID NO: 346 - DNA, 20 bp, Artificial Sequence
  Chemically synthetized sequence
tctcttgttccattgtagta

SEQ ID NO: 347 - DNA, 20 bp, Artificial Sequence
  Chemically synthetized sequence
ttctactgttgggattttgc

SEQ ID NO: 348 - DNA, 20 bp, Artificial Sequence
  Chemically synthetized sequence
ccaagctgccatatggaaaa

SEQ ID NO: 349 - DNA, 20 bp, Artificial Sequence
  Chemically synthetized sequence
attctttaccatcatggcat

SEQ ID NO: 350 - DNA, 20 bp, Artificial Sequence
  Chemically synthetized sequence
gccatcaactccgatatatg

SEQ ID NO: 351 - DNA, 20 bp, Artificial Sequence
  Chemically synthetized sequence
gtgtccttaggatgtttttt

SEQ ID NO: 352 - DNA, 20 bp, Artificial Sequence
  Chemically synthetized sequence
gggccatcagttatcataag

SEQ ID NO: 353 - DNA, 20 bp, Artificial Sequence
  Chemically synthetized sequence
gcattcactaatccctgaag

SEQ ID NO: 354 - DNA, 20 bp, Artificial Sequence
  Chemically synthetized sequence
ccctctcgaatcttaaggaa

SEQ ID NO: 355 - DNA, 20 bp, Artificial Sequence
  Chemically synthetized sequence
tcctcagtatgttttactct

SEQ ID NO: 356 - DNA, 20 bp, Artificial Sequence
  Chemically synthetized sequence
tggttttgttgctggcaaat

SEQ ID NO: 357 - DNA, 20 bp, Artificial Sequence
  Chemically synthetized sequence
aaagggtctttttgctgtgt

SEQ ID NO: 358 - DNA, 20 bp, Artificial Sequence
  Chemically synthetized sequence
atcaatcttatttccgctgt

SEQ ID NO: 359 - DNA, 20 bp, Artificial Sequence
  Chemically synthetized sequence
tctgggggtgtccaaataag

SEQ ID NO: 360 - DNA, 20 bp, Artificial Sequence
  Chemically synthetized sequence
ctgttatgcttccatcattt

SEQ ID NO: 361 - DNA, 20 bp, Artificial Sequence
  Chemically synthetized sequence
atgaacaaatcctcccttga

SEQ ID NO: 362 - DNA, 20 bp, Artificial Sequence
  Chemically synthetized sequence
caatcttggatgccattctt

SEQ ID NO: 363 - DNA, 20 bp, Artificial Sequence
  Chemically synthetized sequence
actttacatacagtcccatc

SEQ ID NO: 364 - DNA, 20 bp, Artificial Sequence
  Chemically synthetized sequence
actaagagcaagggcttcac

SEQ ID NO: 365 - DNA, 20 bp, Artificial Sequence
  Chemically synthetized sequence
gccaaaggaataccaaccag

SEQ ID NO: 366 - DNA, 20 bp, Artificial Sequence
  Chemically synthetized sequence
caggggacatcacatttctt

SEQ ID NO: 367 - DNA, 20 bp, Artificial Sequence
  Chemically synthetized sequence
accatcatgtaccatttcta

SEQ ID NO: 368 - DNA, 20 bp, Artificial Sequence
  Chemically synthetized sequence
tacagagccatatcaacacc

SEQ ID NO: 369 - DNA, 20 bp, Artificial Sequence
  Chemically synthetized sequence
gcattggatgttactaccat

SEQ ID NO: 370 - DNA, 20 bp, Artificial Sequence
  Chemically synthetized sequence
gatgttattccagtgcagat

SEQ ID NO: 371 - DNA, 20 bp, Artificial Sequence
  Chemically synthetized sequence
tgttttgaccacatgaggtg

SEQ ID NO: 372 - DNA, 20 bp, Artificial Sequence
  Chemically synthetized sequence
gtatcacaccagtcacattg

SEQ ID NO: 373 - DNA, 20 bp, Artificial Sequence
  Chemically synthetized sequence
cttcgtggagtattgaagct

SEQ ID NO: 374 - DNA, 20 bp, Artificial Sequence
  Chemically synthetized sequence
aaagcacccggatgtaacag

SEQ ID NO: 375 - DNA, 20 bp, Artificial Sequence
  Chemically synthetized sequence
tgatttttgttctgtcgtgc

SEQ ID NO: 376 - DNA, 20 bp, Artificial Sequence
  Chemically synthetized sequence
tcctctgagaagattgggta

SEQ ID NO: 377 - DNA, 20 bp, Artificial Sequence
  Chemically synthetized sequence
tgggttgatagcctgatatt

SEQ ID NO: 378 - DNA, 20 bp, Artificial Sequence
  Chemically synthetized sequence
ccaagccattgttgcgaaaa

SEQ ID NO: 379 - DNA, 20 bp, Artificial Sequence
  Chemically synthetized sequence
tcttcatttgggttttgtca

SEQ ID NO: 380 - DNA, 20 bp, Artificial Sequence
  Chemically synthetized sequence
tttgaggatttgagtctcca

SEQ ID NO: 381 - DNA, 20 bp, Artificial Sequence
  Chemically synthetized sequence
tggttactccattagcagat

SEQ ID NO: 382 - DNA, 20 bp, Artificial Sequence
  Chemically synthetized sequence
ttctgtttgatctgggaagc

SEQ ID NO: 383 - DNA, 20 bp, Artificial Sequence
  Chemically synthetized sequence
ggtagacaattgttcctgtt

SEQ ID NO: 384 - DNA, 20 bp, Artificial Sequence
  Chemically synthetized sequence
cacaccttttgaggcaacaa

SEQ ID NO: 385 - DNA, 20 bp, Artificial Sequence
  Chemically synthetized sequence
ttttattactttgctcctgc

SEQ ID NO: 386 - DNA, 20 bp, Artificial Sequence
  Chemically synthetized sequence
gcaatctgcttcaccaatta

SEQ ID NO: 387 - DNA, 20 bp, Artificial Sequence
  Chemically synthetized sequence
atccaccgtatttttcatga

SEQ ID NO: 388 - DNA, 20 bp, Artificial Sequence
  Chemically synthetized sequence
aaggtgttttcacccatatt

SEQ ID NO: 389 - DNA, 20 bp, Artificial Sequence
  Chemically synthetized sequence
atagttttgcaggaggtcta

SEQ ID NO: 390 - DNA, 20 bp, Artificial Sequence
  Chemically synthetized sequence
ccttctaggaaaccagcaat

SEQ ID NO: 391 - DNA, 20 bp, Artificial Sequence
  Chemically synthetized sequence
ttcttgcgtactcttaaggt

SEQ ID NO: 392 - DNA, 20 bp, Artificial Sequence
  Chemically synthetized sequence
ctttacttctagctcactca

SEQ ID NO: 393 - DNA, 20 bp, Artificial Sequence
  Chemically synthetized sequence
gtatttcgttgtggagttca

SEQ ID NO: 394 - DNA, 20 bp, Artificial Sequence
  Chemically synthetized sequence
agatcatccactttctcatc

SEQ ID NO: 395 - DNA, 20 bp, Artificial Sequence
  Chemically synthetized sequence
ttgcgagcttatagtgtcag

SEQ ID NO: 396 - DNA, 20 bp, Artificial Sequence
  Chemically synthetized sequence
tccttcgttggaaagcaaga

SEQ ID NO: 397 - DNA, 20 bp, Artificial Sequence
  Chemically synthetized sequence
atttcctatctctacagcag

SEQ ID NO: 398 - DNA, 20 bp, Artificial Sequence
  Chemically synthetized sequence
ttgtgtttggtttcgaagca

SEQ ID NO: 399 - DNA, 20 bp, Artificial Sequence
  Chemically synthetized sequence
acagccaaactagaagcagc

SEQ ID NO: 400 - DNA, 20 bp, Artificial Sequence
  Chemically synthetized sequence
gcatgaaacgttgtctctgg

SEQ ID NO: 401 - DNA, 20 bp, Artificial Sequence
  Chemically synthetized sequence
atcttttggtttggattcat

SEQ ID NO: 402 - DNA, 20 bp, Artificial Sequence
  Chemically synthetized sequence
tacagaccgaaccaatggtt

SEQ ID NO: 403 - DNA, 20 bp, Artificial Sequence
  Chemically synthetized sequence
aagttagccattccaattgt

SEQ ID NO: 404 - DNA, 20 bp, Artificial Sequence
  Chemically synthetized sequence
gtggctaatccatattgaga

SEQ ID NO: 405 - DNA, 20 bp, Artificial Sequence
  Chemically synthetized sequence
tctgattttgattcccaagt

SEQ ID NO: 406 - DNA, 20 bp, Artificial Sequence
  Chemically synthetized sequence
tgacgctttgattgcatgtt

SEQ ID NO: 407 - DNA, 20 bp, Artificial Sequence
  Chemically synthetized sequence
tacccaagtgttgttttcat

SEQ ID NO: 408 - DNA, 20 bp, Artificial Sequence
  Chemically synthetized sequence
gctgatgttaacatatgtct

SEQ ID NO: 409 - DNA, 20 bp, Artificial Sequence
  Chemically synthetized sequence
actgtccagcagcaaagttg

SEQ ID NO: 410 - DNA, 20 bp, Artificial Sequence
  Chemically synthetized sequence
gctaatttcacggaaaccac

SEQ ID NO: 411 - DNA, 20 bp, Artificial Sequence
  Chemically synthetized sequence
agggcagagagaggaattgc

SEQ ID NO: 412 - DNA, 20 bp, Artificial Sequence
  Chemically synthetized sequence
tgtatatagcccatccacta

SEQ ID NO: 413 - DNA, 20 bp, Artificial Sequence
  Chemically synthetized sequence
cttatgacaaacacatcccc

SEQ ID NO: 414 - DNA, 20 bp, Artificial Sequence
  Chemically synthetized sequence
ggggagcatgatatgaatgg

SEQ ID NO: 415 - DNA, 20 bp, Artificial Sequence
  Chemically synthetized sequence
gaagaaggttctgcattcca

SEQ ID NO: 416 - DNA, 20 bp, Artificial Sequence
  Chemically synthetized sequence
tgtttgtcatttagcaaggc

SEQ ID NO: 417 - DNA, 20 bp, Artificial Sequence
  Chemically synthetized sequence
gtctttaatggttccattgg

SEQ ID NO: 418 - DNA, 20 bp, Artificial Sequence
  Chemically synthetized sequence
ttagggttcgatatgggctc

SEQ ID NO: 419 - DNA, 20 bp, Artificial Sequence
  Chemically synthetized sequence
acttcaccaataggacagct

SEQ ID NO: 420 - DNA, 20 bp, Artificial Sequence
  Chemically synthetized sequence
tcttgagttgtatggagagg

SEQ ID NO: 421 - DNA, 20 bp, Artificial Sequence
  Chemically synthetized sequence
acaagcacttgctgaccaag

SEQ ID NO: 422 - DNA, 20 bp, Artificial Sequence
  Chemically synthetized sequence
tactttaacacagccactgc

SEQ ID NO: 423 - DNA, 20 bp, Artificial Sequence
  Chemically synthetized sequence
agtgtctgttattatgccgt

SEQ ID NO: 424 - DNA, 20 bp, Artificial Sequence
  Chemically synthetized sequence
tattgtttctccaactcttg

SEQ ID NO: 425 - DNA, 20 bp, Artificial Sequence
  Chemically synthetized sequence
tcagactcttgtgttctcaa

SEQ ID NO: 426 - DNA, 20 bp, Artificial Sequence
  Chemically synthetized sequence
tccatcggtcattacagtaa

SEQ ID NO: 427 - DNA, 20 bp, Artificial Sequence
  Chemically synthetized sequence
tatgaggcctgtccattact

SEQ ID NO: 428 - DNA, 20 bp, Artificial Sequence
  Chemically synthetized sequence
cccttttctattctgaagat

SEQ ID NO: 429 - DNA, 20 bp, Artificial Sequence
  Chemically synthetized sequence
caggataacaggagcattcc

SEQ ID NO: 430 - DNA, 20 bp, Artificial Sequence
  Chemically synthetized sequence
ttctggttgaaagacaccca

SEQ ID NO: 431 - DNA, 20 bp, Artificial Sequence
  Chemically synthetized sequence
gtatcctatctgatattcca

SEQ ID NO: 432 - DNA, 20 bp, Artificial Sequence
  Chemically synthetized sequence
tggattgtctccgaaaatcc

SEQ ID NO: 433 - DNA, 20 bp, Artificial Sequence
  Chemically synthetized sequence
actgcctgtcttatcattag

SEQ ID NO: 434 - DNA, 20 bp, Artificial Sequence
  Chemically synthetized sequence
ccattagacgatactggacc

SEQ ID NO: 435 - DNA, 20 bp, Artificial Sequence
  Chemically synthetized sequence
tagttctccctatccaaaca

SEQ ID NO: 436 - DNA, 20 bp, Artificial Sequence
  Chemically synthetized sequence
ccgtttcttgaactaatgct

SEQ ID NO: 437 - DNA, 20 bp, Artificial Sequence
  Chemically synthetized sequence
cggatcccaaatcatctcaa

SEQ ID NO: 438 - DNA, 20 bp, Artificial Sequence
  Chemically synthetized sequence
gagaagttattgtctgtccc

SEQ ID NO: 439 - DNA, 20 bp, Artificial Sequence
  Chemically synthetized sequence
tcctacgatatcttgcttta

SEQ ID NO: 440 - DNA, 20 bp, Artificial Sequence
  Chemically synthetized sequence
tatatcctgaccactcattt

SEQ ID NO: 441 - DNA, 20 bp, Artificial Sequence
  Chemically synthetized sequence
ggatgctgaacaaaactccc

SEQ ID NO: 442 - DNA, 20 bp, Artificial Sequence
  Chemically synthetized sequence
gcaaggtcttatacaatcca

SEQ ID NO: 443 - DNA, 20 bp, Artificial Sequence
  Chemically synthetized sequence
cagattgtgttctctttggg

SEQ ID NO: 444 - DNA, 20 bp, Artificial Sequence
  Chemically synthetized sequence
caaaaggatatgctgctccc

SEQ ID NO: 445 - DNA, 20 bp, Artificial Sequence
  Chemically synthetized sequence
cacagtgtcactgtttacac

SEQ ID NO: 446 - DNA, 20 bp, Artificial Sequence
  Chemically synthetized sequence
taaatggcaactcagcaccg

SEQ ID NO: 447 - DNA, 30 bp, Artificial Sequence
  Chemically synthetized sequence
tagtgataattaggggcattcatttcgact

SEQ ID NO: 448 - DNA, 30 bp, Artificial Sequence
  Chemically synthetized sequence
tagtaataattaggggcattcatttcgact

SEQ ID NO: 449 - DNA, 24 bp, Artificial Sequence
  Chemically synthetized sequence
agtcgaaatgaatgcccctaatta

SEQ ID NO: 450 - DNA, 22 bp, Artificial Sequence
  Chemically synthetized sequence
agtcgaaatgaatgcccctaat

SEQ ID NO: 451 - DNA, 20 bp, Artificial Sequence
  Chemically synthetized sequence
agtcgaaatgaatgccccta

SEQ ID NO: 452 - DNA, 20 bp, Artificial Sequence
  Chemically synthetized sequence
aggacggacatccgacatgc

SEQ ID NO: 453 - DNA, 20 bp, Artificial Sequence
  Chemically synthetized sequence
cagcattcagcgggaacaat

SEQ ID NO: 454 - DNA, 20 bp, Artificial Sequence
  Chemically synthetized sequence
cttttgaaagagcaaccgtt

SEQ ID NO: 455 - DNA, 20 bp, Artificial Sequence
  Chemically synthetized sequence
attctcagtgcagcggaatc

SEQ ID NO: 456 - DNA, 20 bp, Artificial Sequence
  Chemically synthetized sequence
aaaccatggactccaatacc

SEQ ID NO: 457 - DNA, 20 bp, Artificial Sequence
  Chemically synthetized sequence
aaagctttccacaagagggg

SEQ ID NO: 458 - DNA, 20 bp, Artificial Sequence
  Chemically synthetized sequence
tcagcctgatgagaccaaat

SEQ ID NO: 459 - DNA, 20 bp, Artificial Sequence
  Chemically synthetized sequence
gtactcactggtcgggatag

SEQ ID NO: 460 - DNA, 20 bp, Artificial Sequence
  Chemically synthetized sequence
ggcatgactttgaaagggaa

SEQ ID NO: 461 - DNA, 20 bp, Artificial Sequence
  Chemically synthetized sequence
cggtcagcactcattctgag

SEQ ID NO: 462 - DNA, 20 bp, Artificial Sequence
  Chemically synthetized sequence
ttgaagacctcattttcctg

SEQ ID NO: 463 - DNA, 20 bp, Artificial Sequence
  Chemically synthetized sequence
cgaaggacaagggttgctta

SEQ ID NO: 464 - DNA, 20 bp, Artificial Sequence
  Chemically synthetized sequence
aggttcaacacttcccagaa

SEQ ID NO: 465 - DNA, 20 bp, Artificial Sequence
  Chemically synthetized sequence
tatgatggacgactaatcca

SEQ ID NO: 466 - DNA, 20 bp, Artificial Sequence
  Chemically synthetized sequence
gatgccacagaaatcagagc

SEQ ID NO: 467 - DNA, 20 bp, Artificial Sequence
  Chemically synthetized sequence
aacttcagacatgagggcag

SEQ ID NO: 468 - DNA, 20 bp, Artificial Sequence
  Chemically synthetized sequence
tcactggaaatacggaggga

SEQ ID NO: 469 - DNA, 20 bp, Artificial Sequence
  Chemically synthetized sequence
aaacctcccatttgaaaagt

SEQ ID NO: 470 - DNA, 20 bp, Artificial Sequence
  Chemically synthetized sequence
aacctacgttttctgtacaa

SEQ ID NO: 471 - DNA, 20 bp, Artificial Sequence
  Chemically synthetized sequence
aatatgggatcgagcactct

SEQ ID NO: 472 - DNA, 20 bp, Artificial Sequence
  Chemically synthetized sequence
tcgggggaaactgtcaacta

SEQ ID NO: 473 - DNA, 20 bp, Artificial Sequence
  Chemically synthetized sequence
taagcttcatcagagggaca

SEQ ID NO: 474 - DNA, 20 bp, Artificial Sequence
  Chemically synthetized sequence
acagcttaatcaggcctaac

SEQ ID NO: 475 - DNA, 20 bp, Artificial Sequence
  Chemically synthetized sequence
gttattccttggtgggaata

SEQ ID NO: 476 - DNA, 20 bp, Artificial Sequence
  Chemically synthetized sequence
tttagcaagatctgcactga

SEQ ID NO: 477 - DNA, 20 bp, Artificial Sequence
  Chemically synthetized sequence
gaggtgagaatgggcggaaa

SEQ ID NO: 478 - DNA, 20 bp, Artificial Sequence
  Chemically synthetized sequence
atcgggacaatggtgatgga

SEQ ID NO: 479 - DNA, 20 bp, Artificial Sequence
  Chemically synthetized sequence
gtgatcatgaaggtcggttg

SEQ ID NO: 480 - DNA, 20 bp, Artificial Sequence
  Chemically synthetized sequence
caactgagattagggcatcc

SEQ ID NO: 481 - DNA, 20 bp, Artificial Sequence
  Chemically synthetized sequence
aaaatcatggcgtcccaagg

SEQ ID NO: 482 - DNA, 20 bp, Artificial Sequence
  Chemically synthetized sequence
taatcactcactgagtgaca

SEQ ID NO: 483 - DNA, 20 bp, Artificial Sequence
  Chemically synthetized sequence
tacgtttggaatgtgggtgt

SEQ ID NO: 484 - DNA, 20 bp, Artificial Sequence
  Chemically synthetized sequence
gacactatgactgtgattgt

SEQ ID NO: 485 - DNA, 20 bp, Artificial Sequence
  Chemically synthetized sequence
tgacctcgattattaaggca

SEQ ID NO: 486 - DNA, 20 bp, Artificial Sequence
  Chemically synthetized sequence
accagtggaaatgctttcat

SEQ ID NO: 487 - DNA, 20 bp, Artificial Sequence
  Chemically synthetized sequence
ggacgtgatgcagatgtcaa

SEQ ID NO: 488 - DNA, 20 bp, Artificial Sequence
  Chemically synthetized sequence
tgctctaagcaagcaagctg

SEQ ID NO: 489 - DNA, 20 bp, Artificial Sequence
  Chemically synthetized sequence
tgtttgcagtggaaagacct

SEQ ID NO: 490 - DNA, 20 bp, Artificial Sequence
  Chemically synthetized sequence
tctggccaaataagctgcag

SEQ ID NO: 491 - DNA, 20 bp, Artificial Sequence
  Chemically synthetized sequence
atctggggggaacgaagtag

SEQ ID NO: 492 - DNA, 20 bp, Artificial Sequence
  Chemically synthetized sequence
ctctgatgtccatcaagctc

SEQ ID NO: 493 - DNA, 20 bp, Artificial Sequence
  Chemically synthetized sequence
caggtagaaggaatgggagc

SEQ ID NO: 494 - DNA, 20 bp, Artificial Sequence
  Chemically synthetized sequence
aatgcaagggtttccatgtt

SEQ ID NO: 495 - DNA, 20 bp, Artificial Sequence
  Chemically synthetized sequence
ttcaagcctagatcagcatt

SEQ ID NO: 496 - DNA, 20 bp, Artificial Sequence
  Chemically synthetized sequence
gtctgcattaacaggcacag

SEQ ID NO: 497 - DNA, 20 bp, Artificial Sequence
  Chemically synthetized sequence
cctatgaagacctgagagtt

SEQ ID NO: 498 - DNA, 20 bp, Artificial Sequence
  Chemically synthetized sequence
attcttcatgtcttgcttcg

SEQ ID NO: 499 - DNA, 20 bp, Artificial Sequence
  Chemically synthetized sequence
caacacctgtgtccatatta

SEQ ID NO: 500 - DNA, 20 bp, Artificial Sequence
  Chemically synthetized sequence
aagccatggctctttacaat

SEQ ID NO: 501 - DNA, 20 bp, Artificial Sequence
  Chemically synthetized sequence
gagtactctatggttgggta

SEQ ID NO: 502 - DNA, 20 bp, Artificial Sequence
  Chemically synthetized sequence
cctcaactagggttcaatgt

SEQ ID NO: 503 - DNA, 20 bp, Artificial Sequence
  Chemically synthetized sequence
ccataagcatttacgccaaa

SEQ ID NO: 504 - DNA, 20 bp, Artificial Sequence
  Chemically synthetized sequence
ctgtggcaagcaaagtggtg

SEQ ID NO: 505 - DNA, 20 bp, Artificial Sequence
  Chemically synthetized sequence
tcgtagtatggtcgttgtta

SEQ ID NO: 506 - DNA, 20 bp, Artificial Sequence
  Chemically synthetized sequence
ttgaagatctaaccctgctt

SEQ ID NO: 507 - DNA, 20 bp, Artificial Sequence
  Chemically synthetized sequence
gatcggaagcagaaatccgg

SEQ ID NO: 508 - DNA, 20 bp, Artificial Sequence
  Chemically synthetized sequence
cccaacaaaaggctctagtt

SEQ ID NO: 509 - DNA, 20 bp, Artificial Sequence
  Chemically synthetized sequence
agccaagactgcctatgaaa

SEQ ID NO: 510 - DNA, 20 bp, Artificial Sequence
  Chemically synthetized sequence
cagacagagggctattgaga

SEQ ID NO: 511 - DNA, 20 bp, Artificial Sequence
  Chemically synthetized sequence
gctgaagccattcgatttat

SEQ ID NO: 512 - DNA, 20 bp, Artificial Sequence
  Chemically synthetized sequence
aatcaaaggaggtggaacct

SEQ ID NO: 513 - DNA, 20 bp, Artificial Sequence
  Chemically synthetized sequence
aatcagtacctttgcgggaa

SEQ ID NO: 514 - DNA, 20 bp, Artificial Sequence
  Chemically synthetized sequence
agagttggacttgatccttc

SEQ ID NO: 515 - DNA, 20 bp, Artificial Sequence
  Chemically synthetized sequence
atgaatgatgtctgtttcca

SEQ ID NO: 516 - DNA, 20 bp, Artificial Sequence
  Chemically synthetized sequence
tgatggcttcagtggactaa

SEQ ID NO: 517 - DNA, 20 bp, Artificial Sequence
  Chemically synthetized sequence
atctacttcagccctataag

SEQ ID NO: 518 - DNA, 20 bp, Artificial Sequence
  Chemically synthetized sequence
aagaattctattggctgcca

SEQ ID NO: 519 - DNA, 20 bp, Artificial Sequence
  Chemically synthetized sequence
taatccaaaatgcgcatgcc

SEQ ID NO: 520 - DNA, 20 bp, Artificial Sequence
  Chemically synthetized sequence
tgatggtcaaagctggactc

SEQ ID NO: 521 - DNA, 20 bp, Artificial Sequence
  Chemically synthetized sequence
ctgggcgaattctataacca

SEQ ID NO: 522 - DNA, 20 bp, Artificial Sequence
  Chemically synthetized sequence
agagcgtctacaacatggtg

SEQ ID NO: 523 - DNA, 20 bp, Artificial Sequence
  Chemically synthetized sequence
cagaccccgacagagataaa

SEQ ID NO: 524 - DNA, 20 bp, Artificial Sequence
  Chemically synthetized sequence
gcaaccacaagcagtgaaga

SEQ ID NO: 525 - DNA, 20 bp, Artificial Sequence
  Chemically synthetized sequence
cccaccaagcaacaaacgaa

SEQ ID NO: 526 - DNA, 20 bp, Artificial Sequence
  Chemically synthetized sequence
agaccaatcattagaccagc

SEQ ID NO: 527 - DNA, 20 bp, Artificial Sequence
  Chemically synthetized sequence
taacttctggaaccagtggg

SEQ ID NO: 528 - DNA, 20 bp, Artificial Sequence
  Chemically synthetized sequence
acggtataaacactgggaca

SEQ ID NO: 529 - DNA, 20 bp, Artificial Sequence
  Chemically synthetized sequence
tgaacttcaagcaccagtaa

SEQ ID NO: 530 - DNA, 20 bp, Artificial Sequence
  Chemically synthetized sequence
gcagaagcacagcattttct

SEQ ID NO: 531 - DNA, 20 bp, Artificial Sequence
  Chemically synthetized sequence
atcgtttggtgccttgagac

SEQ ID NO: 532 - DNA, 20 bp, Artificial Sequence
  Chemically synthetized sequence
ccagtctccatttgttcata

SEQ ID NO: 533 - DNA, 20 bp, Artificial Sequence
  Chemically synthetized sequence
ctgatttctgtggcatcctg

SEQ ID NO: 534 - DNA, 20 bp, Artificial Sequence
  Chemically synthetized sequence
ccaatcattcttccgacaga

SEQ ID NO: 535 - DNA, 20 bp, Artificial Sequence
  Chemically synthetized sequence
gatgtagaatctcccgattc

SEQ ID NO: 536 - DNA, 20 bp, Artificial Sequence
  Chemically synthetized sequence
gtttgagttcagtgcacatt

SEQ ID NO: 537 - DNA, 20 bp, Artificial Sequence
  Chemically synthetized sequence
agtcgtccatcataatcact

SEQ ID NO: 538 - DNA, 20 bp, Artificial Sequence
  Chemically synthetized sequence
ctattgttatgctattctgg

SEQ ID NO: 539 - DNA, 20 bp, Artificial Sequence
  Chemically synthetized sequence
aaagcagaaagcaccatcct

SEQ ID NO: 540 - DNA, 20 bp, Artificial Sequence
  Chemically synthetized sequence
atatgggtcctcctgttttc

SEQ ID NO: 541 - DNA, 20 bp, Artificial Sequence
  Chemically synthetized sequence
cactttccgtctactcttct

SEQ ID NO: 542 - DNA, 20 bp, Artificial Sequence
  Chemically synthetized sequence
attgtttgcttggcgccaaa

SEQ ID NO: 543 - DNA, 20 bp, Artificial Sequence
  Chemically synthetized sequence
atgagtaagacctgctgttg

SEQ ID NO: 544 - DNA, 20 bp, Artificial Sequence
  Chemically synthetized sequence
atatgtggcatcattcaggt

SEQ ID NO: 545 - DNA, 20 bp, Artificial Sequence
  Chemically synthetized sequence
gaacaagcgctcttgttctc

SEQ ID NO: 546 - DNA, 20 bp, Artificial Sequence
  Chemically synthetized sequence
ttagagagcacattctggga

SEQ ID NO: 547 - DNA, 20 bp, Artificial Sequence
  Chemically synthetized sequence
ctgggaagtgttgaaccttg

SEQ ID NO: 548 - DNA, 20 bp, Artificial Sequence
  Chemically synthetized sequence
tattgttccaactcctttca

SEQ ID NO: 549 - DNA, 20 bp, Artificial Sequence
  Chemically synthetized sequence
tcattctgattaactccatt

SEQ ID NO: 550 - DNA, 20 bp, Artificial Sequence
  Chemically synthetized sequence
cggtcattgattccacgttt

SEQ ID NO: 551 - DNA, 20 bp, Artificial Sequence
  Chemically synthetized sequence
attttcacccctccagaaat

SEQ ID NO: 552 - DNA, 20 bp, Artificial Sequence
  Chemically synthetized sequence
ataagcaacccttgtccttc

SEQ ID NO: 553 - DNA, 20 bp, Artificial Sequence
  Chemically synthetized sequence
ttacttgatccatcattgcc

SEQ ID NO: 554 - DNA, 20 bp, Artificial Sequence
  Chemically synthetized sequence
tttcctgggtttcgactttc

SEQ ID NO: 555 - DNA, 20 bp, Artificial Sequence
  Chemically synthetized sequence
aatgaggtcttcaatctcag

SEQ ID NO: 556 - DNA, 20 bp, Artificial Sequence
  Chemically synthetized sequence
agaatgagtgctgaccgtgc

SEQ ID NO: 557 - DNA, 20 bp, Artificial Sequence
  Chemically synthetized sequence
tttatgtgcaactgatcccc

SEQ ID NO: 558 - DNA, 20 bp, Artificial Sequence
  Chemically synthetized sequence
ctactgcaagcccatacaca

SEQ ID NO: 559 - DNA, 20 bp, Artificial Sequence
  Chemically synthetized sequence
cttccctttcaaagtcatgc

SEQ ID NO: 560 - DNA, 20 bp, Artificial Sequence
  Chemically synthetized sequence
ctcatcaggctgaccacttg

SEQ ID NO: 561 - DNA, 20 bp, Artificial Sequence
  Chemically synthetized sequence
gtgagctgggttttcatttg

SEQ ID NO: 562 - DNA, 20 bp, Artificial Sequence
  Chemically synthetized sequence
ccatccacaccaattgactc

SEQ ID NO: 563 - DNA, 20 bp, Artificial Sequence
  Chemically synthetized sequence
cttcaaatgcagcagagtgg

SEQ ID NO: 564 - DNA, 20 bp, Artificial Sequence
  Chemically synthetized sequence
cactttctttcctcttatga

SEQ ID NO: 565 - DNA, 20 bp, Artificial Sequence
  Chemically synthetized sequence
tgtggaaagctttcctcttg

SEQ ID NO: 566 - DNA, 20 bp, Artificial Sequence
  Chemically synthetized sequence
atttgaagcaatctggaccc

SEQ ID NO: 567 - DNA, 20 bp, Artificial Sequence
  Chemically synthetized sequence
gtattggagtccatggtttc

SEQ ID NO: 568 - DNA, 20 bp, Artificial Sequence
  Chemically synthetized sequence
cagtatctgcttctcagttc

SEQ ID NO: 569 - DNA, 20 bp, Artificial Sequence
  Chemically synthetized sequence
tgccttttgttgattggtat

SEQ ID NO: 570 - DNA, 20 bp, Artificial Sequence
  Chemically synthetized sequence
actgagaatgtaggctgcac

SEQ ID NO: 571 - DNA, 20 bp, Artificial Sequence
  Chemically synthetized sequence
ttcaaaagggagattccgct

SEQ ID NO: 572 - DNA, 20 bp, Artificial Sequence
  Chemically synthetized sequence
gaatgctgccataacggttg

SEQ ID NO: 573 - DNA, 20 bp, Artificial Sequence
  Chemically synthetized sequence
atgtccgtccttcattgttc

SEQ ID NO: 574 - DNA, 20 bp, Artificial Sequence
  Chemically synthetized sequence
caaatcttctggctttgcac

SEQ ID NO: 575 - DNA, 20 bp, Artificial Sequence
  Chemically synthetized sequence
tttcgtccgagagctcgaag

SEQ ID NO: 576 - DNA, 20 bp, Artificial Sequence
  Chemically synthetized sequence
aaaggaaggcacgatcgggt

SEQ ID NO: 577 - DNA, 20 bp, Artificial Sequence
  Chemically synthetized sequence
aagacccttcattactcatg

SEQ ID NO: 578 - DNA, 20 bp, Artificial Sequence
  Chemically synthetized sequence
tctgcattgtctccgaagaa

SEQ ID NO: 579 - DNA, 20 bp, Artificial Sequence
  Chemically synthetized sequence
gaaaatgctgtgcttctgct

SEQ ID NO: 580 - DNA, 20 bp, Artificial Sequence
  Chemically synthetized sequence
agttcttttactggtgcttg

SEQ ID NO: 581 - DNA, 20 bp, Artificial Sequence
  Chemically synthetized sequence
ccagtgtttataccgtcaat

SEQ ID NO: 582 - DNA, 20 bp, Artificial Sequence
  Chemically synthetized sequence
ttccggtgttttgtcaattg

SEQ ID NO: 583 - DNA, 20 bp, Artificial Sequence
  Chemically synthetized sequence
cactggttccagaagttatt

SEQ ID NO: 584 - DNA, 20 bp, Artificial Sequence
  Chemically synthetized sequence
aagggttgctggtctaatga

SEQ ID NO: 585 - DNA, 20 bp, Artificial Sequence
  Chemically synthetized sequence
gacatcatcttcactgcttg

SEQ ID NO: 586 - DNA, 20 bp, Artificial Sequence
  Chemically synthetized sequence
ctcttctttatctctgtcgg

SEQ ID NO: 587 - DNA, 20 bp, Artificial Sequence
  Chemically synthetized sequence
tttcaccaccatgttgtaga

SEQ ID NO: 588 - DNA, 20 bp, Artificial Sequence
  Chemically synthetized sequence
ctttgaccatcatctggtta

SEQ ID NO: 589 - DNA, 20 bp, Artificial Sequence
  Chemically synthetized sequence
ttctctccatgtcatcattg

SEQ ID NO: 590 - DNA, 20 bp, Artificial Sequence
  Chemically synthetized sequence
gcatgcgcattttggattag

SEQ ID NO: 591 - DNA, 20 bp, Artificial Sequence
  Chemically synthetized sequence
agccaatagaattctttcca

SEQ ID NO: 592 - DNA, 20 bp, Artificial Sequence
  Chemically synthetized sequence
cggttttcttgtcatcagtg

SEQ ID NO: 593 - DNA, 20 bp, Artificial Sequence
  Chemically synthetized sequence
tcatctcttaccatcttgta

SEQ ID NO: 594 - DNA, 20 bp, Artificial Sequence
  Chemically synthetized sequence
agggctgaagtagatggttt

SEQ ID NO: 595 - DNA, 20 bp, Artificial Sequence
  Chemically synthetized sequence
gtgatttagtccactgaagc

SEQ ID NO: 596 - DNA, 20 bp, Artificial Sequence
  Chemically synthetized sequence
atctgtgaatgcccaatcat

SEQ ID NO: 597 - DNA, 20 bp, Artificial Sequence
  Chemically synthetized sequence
gtgcctttgatctttggaaa

SEQ ID NO: 598 - DNA, 20 bp, Artificial Sequence
  Chemically synthetized sequence
ggatcaagtccaactctttt

SEQ ID NO: 599 - DNA, 20 bp, Artificial Sequence
  Chemically synthetized sequence
cccgcaaaggtactgattaa

SEQ ID NO: 600 - DNA, 20 bp, Artificial Sequence
  Chemically synthetized sequence
ctttgattgcaacaccagtc

SEQ ID NO: 601 - DNA, 20 bp, Artificial Sequence
  Chemically synthetized sequence
gaatggcttcagccactaag

SEQ ID NO: 602 - DNA, 20 bp, Artificial Sequence
  Chemically synthetized sequence
gccattgctcttcctataaa

SEQ ID NO: 603 - DNA, 20 bp, Artificial Sequence
  Chemically synthetized sequence
tggctttgatgtctctcaat

SEQ ID NO: 604 - DNA, 20 bp, Artificial Sequence
  Chemically synthetized sequence
cacttgatcaactagagcct

SEQ ID NO: 605 - DNA, 20 bp, Artificial Sequence
  Chemically synthetized sequence
caagcagggttagatcttca

SEQ ID NO: 606 - DNA, 20 bp, Artificial Sequence
  Chemically synthetized sequence
gcctaacaacgaccatacta

SEQ ID NO: 607 - DNA, 20 bp, Artificial Sequence
  Chemically synthetized sequence
ttatgggaagcaccactttg

SEQ ID NO: 608 - DNA, 20 bp, Artificial Sequence
  Chemically synthetized sequence
tagttgaggtattttggcgt

SEQ ID NO: 609 - DNA, 20 bp, Artificial Sequence
  Chemically synthetized sequence
ccaaccatagagtactcttc

SEQ ID NO: 610 - DNA, 20 bp, Artificial Sequence
  Chemically synthetized sequence
ttgtaaagagccatggcttc

SEQ ID NO: 611 - DNA, 20 bp, Artificial Sequence
  Chemically synthetized sequence
aatatggacacaggtgttgc

SEQ ID NO: 612 - DNA, 20 bp, Artificial Sequence
  Chemically synthetized sequence
ttatcttttgcatcatctcc

SEQ ID NO: 613 - DNA, 20 bp, Artificial Sequence
  Chemically synthetized sequence
gctccgaagcaagacatgaa

SEQ ID NO: 614 - DNA, 20 bp, Artificial Sequence
  Chemically synthetized sequence
aactctcaggtcttcatagg

SEQ ID NO: 615 - DNA, 20 bp, Artificial Sequence
  Chemically synthetized sequence
cttgaattctgtgcctgtta

SEQ ID NO: 616 - DNA, 20 bp, Artificial Sequence
  Chemically synthetized sequence
aacccttgcattttaatgct

SEQ ID NO: 617 - DNA, 20 bp, Artificial Sequence
  Chemically synthetized sequence
tgttcctttgctggaacatg

SEQ ID NO: 618 - DNA, 20 bp, Artificial Sequence
  Chemically synthetized sequence
tggagcttgatggacatcag

SEQ ID NO: 619 - DNA, 20 bp, Artificial Sequence
  Chemically synthetized sequence
tctttccactgcaaacactg

SEQ ID NO: 620 - DNA, 20 bp, Artificial Sequence
  Chemically synthetized sequence
cttgcttgcttagagcaata

SEQ ID NO: 621 - DNA, 20 bp, Artificial Sequence
  Chemically synthetized sequence
tcattgacagcattcttctt

SEQ ID NO: 622 - DNA, 20 bp, Artificial Sequence
  Chemically synthetized sequence
tttgacatctgcatcacgtc

SEQ ID NO: 623 - DNA, 20 bp, Artificial Sequence
  Chemically synthetized sequence
tcttagccattgagtcattc

SEQ ID NO: 624 - DNA, 20 bp, Artificial Sequence
  Chemically synthetized sequence
atgaaagcatttccactggt

SEQ ID NO: 625 - DNA, 20 bp, Artificial Sequence
  Chemically synthetized sequence
tagtgtctattttgttgcct

SEQ ID NO: 626 - DNA, 20 bp, Artificial Sequence
  Chemically synthetized sequence
cccacattccaaacgtattg

SEQ ID NO: 627 - DNA, 20 bp, Artificial Sequence
  Chemically synthetized sequence
gtcactcagtgagtgattat

SEQ ID NO: 628 - DNA, 20 bp, Artificial Sequence
  Chemically synthetized sequence
cttgggacgccatgattttg

SEQ ID NO: 629 - DNA, 20 bp, Artificial Sequence
  Chemically synthetized sequence
tgttcataagaccgtttggt

SEQ ID NO: 630 - DNA, 20 bp, Artificial Sequence
  Chemically synthetized sequence
tggcgatctccatcagtttc

SEQ ID NO: 631 - DNA, 20 bp, Artificial Sequence
  Chemically synthetized sequence
tgccctaatctcagttgcat

SEQ ID NO: 632 - DNA, 20 bp, Artificial Sequence
  Chemically synthetized sequence
catcaatcatcttcccgacg

SEQ ID NO: 633 - DNA, 20 bp, Artificial Sequence
  Chemically synthetized sequence
tttggatgtagaatctccca

SEQ ID NO: 634 - DNA, 20 bp, Artificial Sequence
  Chemically synthetized sequence
ctgagtttaagttcagtgca

SEQ ID NO: 635 - DNA, 20 bp, Artificial Sequence
  Chemically synthetized sequence
gatcaaccgaccttcatgat

SEQ ID NO: 636 - DNA, 20 bp, Artificial Sequence
  Chemically synthetized sequence
agcagagagtaccattttct

SEQ ID NO: 637 - DNA, 20 bp, Artificial Sequence
  Chemically synthetized sequence
cagttttcttgggatctttc

SEQ ID NO: 638 - DNA, 20 bp, Artificial Sequence
  Chemically synthetized sequence
tcgactctcctatatatggg

SEQ ID NO: 639 - DNA, 20 bp, Artificial Sequence
  Chemically synthetized sequence
taaaggacgagttccctcat

SEQ ID NO: 640 - DNA, 20 bp, Artificial Sequence
  Chemically synthetized sequence
atgtagcatcctcaccattg

SEQ ID NO: 641 - DNA, 20 bp, Artificial Sequence
  Chemically synthetized sequence
gccaaatcattatgtgagtt

SEQ ID NO: 642 - DNA, 20 bp, Artificial Sequence
  Chemically synthetized sequence
ccattccagttcgaacaaga

SEQ ID NO: 643 - DNA, 20 bp, Artificial Sequence
  Chemically synthetized sequence
catcagagagcacattctgg

SEQ ID NO: 644 - DNA, 20 bp, Artificial Sequence
  Chemically synthetized sequence
accttctagggagagtcgag

SEQ ID NO: 645 - DNA, 20 bp, Artificial Sequence
  Chemically synthetized sequence
gtttgaccattctgatcagt

SEQ ID NO: 646 - DNA, 20 bp, Artificial Sequence
  Chemically synthetized sequence
aaatttcgatcgttgatccc

SEQ ID NO: 647 - DNA, 20 bp, Artificial Sequence
  Chemically synthetized sequence
taagcacttcttgttttccg

SEQ ID NO: 648 - DNA, 20 bp, Artificial Sequence
  Chemically synthetized sequence
tctttgtgcagctgtttgaa

SEQ ID NO: 649 - DNA, 20 bp, Artificial Sequence
  Chemically synthetized sequence
tttctctaacttgatccacc

SEQ ID NO: 650 - DNA, 20 bp, Artificial Sequence
  Chemically synthetized sequence
tgagatcttcgatctcagcg

SEQ ID NO: 651 - DNA, 20 bp, Artificial Sequence
  Chemically synthetized sequence
tcagtgcagatcttgctaaa

SEQ ID NO: 652 - DNA, 20 bp, Artificial Sequence
  Chemically synthetized sequence
tgagcaactgatcctctcaa

SEQ ID NO: 653 - DNA, 20 bp, Artificial Sequence
  Chemically synthetized sequence
acaggcaggtaggcaagatt

SEQ ID NO: 654 - DNA, 20 bp, Artificial Sequence
  Chemically synthetized sequence
tggatactgcaggtccatac

SEQ ID NO: 655 - DNA, 20 bp, Artificial Sequence
  Chemically synthetized sequence
ctctttttcgaagtcgtacc

SEQ ID NO: 656 - DNA, 20 bp, Artificial Sequence
  Chemically synthetized sequence
tctattcccaccaaggaata

SEQ ID NO: 657 - DNA, 20 bp, Artificial Sequence
  Chemically synthetized sequence
ggctattttgaagtagtttg

SEQ ID NO: 658 - DNA, 20 bp, Artificial Sequence
  Chemically synthetized sequence
ttaggcctgattaagctgta

SEQ ID NO: 659 - DNA, 20 bp, Artificial Sequence
  Chemically synthetized sequence
tgactcttgtgtgctggatt

SEQ ID NO: 660 - DNA, 20 bp, Artificial Sequence
  Chemically synthetized sequence
tccctctgatgaagcttaac

SEQ ID NO: 661 - DNA, 20 bp, Artificial Sequence
  Chemically synthetized sequence
tgtactcctctagttgacag

SEQ ID NO: 662 - DNA, 20 bp, Artificial Sequence
  Chemically synthetized sequence
ccatattatccatgttctca

SEQ ID NO: 663 - DNA, 20 bp, Artificial Sequence
  Chemically synthetized sequence
ttctcagttcaagagtgctc

SEQ ID NO: 664 - DNA, 20 bp, Artificial Sequence
  Chemically synthetized sequence
cctctgttgattagtgtttc

SEQ ID NO: 665 - DNA, 20 bp, Artificial Sequence
  Chemically synthetized sequence
gtaggttgcacactggtttg

SEQ ID NO: 666 - DNA, 20 bp, Artificial Sequence
  Chemically synthetized sequence
tgggaggtttctttgtacag

SEQ ID NO: 667 - DNA, 20 bp, Artificial Sequence
  Chemically synthetized sequence
ccatgatggttgacttttca

SEQ ID NO: 668 - DNA, 20 bp, Artificial Sequence
  Chemically synthetized sequence
tccgtatttccagtgaatgc

SEQ ID NO: 669 - DNA, 20 bp, Artificial Sequence
  Chemically synthetized sequence
cctcatgtctgaagttcttc

SEQ ID NO: 670 - DNA, 20 bp, Artificial Sequence
  Chemically synthetized sequence
ccatcatccttatgatttct

SEQ ID NO: 671 - DNA, 20 bp, Artificial Sequence
  Chemically synthetized sequence
ggaatgacacttcttctggt

SEQ ID NO: 672 - DNA, 20 bp, Artificial Sequence
  Chemically synthetized sequence
tctcgtctgagagctcgaaa

SEQ ID NO: 673 - DNA, 20 bp, Artificial Sequence
  Chemically synthetized sequence
tcgtactcttctgcattgtc

SEQ ID NO: 674 - DNA, 20 bp, Artificial Sequence
  Chemically synthetized sequence
gcagaactactagtattgcc

SEQ ID NO: 675 - DNA, 20 bp, Artificial Sequence
  Chemically synthetized sequence
tgcatttgcggttgcaaatg

SEQ ID NO: 676 - DNA, 20 bp, Artificial Sequence
  Chemically synthetized sequence
attgttcgcatgataaccta

SEQ ID NO: 677 - DNA, 20 bp, Artificial Sequence
  Chemically synthetized sequence
tactgtgtctacagtgtctg

SEQ ID NO: 678 - DNA, 20 bp, Artificial Sequence
  Chemically synthetized sequence
tgcttgtcttctagaaggtt

SEQ ID NO: 679 - DNA, 20 bp, Artificial Sequence
  Chemically synthetized sequence
tcttagtttgcatagtttcc

SEQ ID NO: 680 - DNA, 20 bp, Artificial Sequence
  Chemically synthetized sequence
ccaaatgcaatggggctacc

SEQ ID NO: 681 - DNA, 20 bp, Artificial Sequence
  Chemically synthetized sequence
cagccagcaatgttacattt

SEQ ID NO: 682 - DNA, 20 bp, Artificial Sequence
  Chemically synthetized sequence
tgctgtggagagtgattcac

SEQ ID NO: 683 - DNA, 20 bp, Artificial Sequence
  Chemically synthetized sequence
ccacaatgtaggaccatgag

SEQ ID NO: 684 - DNA, 20 bp, Artificial Sequence
  Chemically synthetized sequence
ccattgtctgaactaggtgt

SEQ ID NO: 685 - DNA, 20 bp, Artificial Sequence
  Chemically synthetized sequence
gaaatctcctgggtaacacg

SEQ ID NO: 686 - DNA, 20 bp, Artificial Sequence
  Chemically synthetized sequence
tctcttagctcctcataatc

SEQ ID NO: 687 - DNA, 20 bp, Artificial Sequence
  Chemically synthetized sequence
acttgtcttggggaatatct

SEQ ID NO: 688 - DNA, 20 bp, Artificial Sequence
  Chemically synthetized sequence
cctttgttcgagtcatgatt

SEQ ID NO: 689 - DNA, 20 bp, Artificial Sequence
  Chemically synthetized sequence
atgtaggatttgctgagctt

SEQ ID NO: 690 - DNA, 20 bp, Artificial Sequence
  Chemically synthetized sequence
catagcacgaggacttcttt

SEQ ID NO: 691 - DNA, 20 bp, Artificial Sequence
  Chemically synthetized sequence
gcactagtagatggatggtg

SEQ ID NO: 692 - DNA, 20 bp, Artificial Sequence
  Chemically synthetized sequence
ctgcattctgatagagactt

SEQ ID NO: 693 - DNA, 20 bp, Artificial Sequence
  Chemically synthetized sequence
gaccccacaaaaacatatgc

SEQ ID NO: 694 - DNA, 20 bp, Artificial Sequence
  Chemically synthetized sequence
ttattgctatttccggcttg

SEQ ID NO: 695 - DNA, 20 bp, Artificial Sequence
  Chemically synthetized sequence
cttcttgatccctcactttg

SEQ ID NO: 696 - DNA, 20 bp, Artificial Sequence
  Chemically synthetized sequence
gtccagtaatagttcattct

SEQ ID NO: 697 - DNA, 20 bp, Artificial Sequence
  Chemically synthetized sequence
gaatgttattttgtctcccg

SEQ ID NO: 698 - DNA, 20 bp, Artificial Sequence
  Chemically synthetized sequence
taccactagatttccagttg

SEQ ID NO: 699 - DNA, 20 bp, Artificial Sequence
  Chemically synthetized sequence
ccattgcgaatgcatatctc

SEQ ID NO: 700 - DNA, 20 bp, Artificial Sequence
  Chemically synthetized sequence
aataccagatccagcatttc

SEQ ID NO: 701 - DNA, 20 bp, Artificial Sequence
  Chemically synthetized sequence
caatcgtggactggtgtatc

SEQ ID NO: 702 - DNA, 20 bp, Artificial Sequence
  Chemically synthetized sequence
ttgggtgtttgacaagttgt

SEQ ID NO: 703 - DNA, 20 bp, Artificial Sequence
  Chemically synthetized sequence
aaatgggaggctggtgttta

SEQ ID NO: 704 - DNA, 20 bp, Artificial Sequence
  Chemically synthetized sequence
cattttccaattgtgatcgg

SEQ ID NO: 705 - DNA, 20 bp, Artificial Sequence
  Chemically synthetized sequence
tggccagtctcaattttgtg

SEQ ID NO: 706 - DNA, 20 bp, Artificial Sequence
  Chemically synthetized sequence
agacgggatattcctcaatc

SEQ ID NO: 707 - DNA, 20 bp, Artificial Sequence
  Chemically synthetized sequence
atggtgataaccgtaccatc

SEQ ID NO: 708 - DNA, 20 bp, Artificial Sequence
  Chemically synthetized sequence
atctcgtcaatggcattctg

SEQ ID NO: 709 - DNA, 20 bp, Artificial Sequence
  Chemically synthetized sequence
tctttacctactgctgtgaa

SEQ ID NO: 710 - DNA, 20 bp, Artificial Sequence
  Chemically synthetized sequence
acagttcggcattgtaagtc

SEQ ID NO: 711 - DNA, 20 bp, Artificial Sequence
  Chemically synthetized sequence
cttcacatttgaatcgtggt

SEQ ID NO: 712 - DNA, 20 bp, Artificial Sequence
  Chemically synthetized sequence
ccaatttccttggcattgtt

SEQ ID NO: 713 - DNA, 20 bp, Artificial Sequence
  Chemically synthetized sequence
ggtaaaattcaaagcagccg

SEQ ID NO: 714 - DNA, 20 bp, Artificial Sequence
  Chemically synthetized sequence
ttttgacactttccatgcac

SEQ ID NO: 715 - DNA, 20 bp, Artificial Sequence
  Chemically synthetized sequence
ttttgggtagtcataagtcc

SEQ ID NO: 716 - DNA, 20 bp, Artificial Sequence
  Chemically synthetized sequence
tgttgattccagctttaccc

SEQ ID NO: 717 - DNA, 20 bp, Artificial Sequence
  Chemically synthetized sequence
tcgccaaaatctggtaaatc

SEQ ID NO: 718 - DNA, 20 bp, Artificial Sequence
  Chemically synthetized sequence
gaactggcgacagttgaata

SEQ ID NO: 719 - DNA, 20 bp, Artificial Sequence
  Chemically synthetized sequence
cagggagactaccagtacca

SEQ ID NO: 720 - DNA, 20 bp, Artificial Sequence
  Chemically synthetized sequence
acatccagaaactgattgcc

SEQ ID NO: 721 - DNA, 20 bp, Artificial Sequence
  Chemically synthetized sequence
cactgtagagacccattaga

SEQ ID NO: 722 - DNA, 20 bp, Artificial Sequence
  Chemically synthetized sequence
ccagacatagaatgtagctc

SEQ ID NO: 723 - DNA, 20 bp, Artificial Sequence
  Chemically synthetized sequence
ttccaggaagtttttgagcg

SEQ ID NO: 724 - DNA, 20 bp, Artificial Sequence
  Chemically synthetized sequence
tttcactatcgttccgtttg

SEQ ID NO: 725 - DNA, 20 bp, Artificial Sequence
  Chemically synthetized sequence
acttcaattcggtcattcgt

SEQ ID NO: 726 - DNA, 20 bp, Artificial Sequence
  Chemically synthetized sequence
aggactgtcgcatatttcac

SEQ ID NO: 727 - DNA, 20 bp, Artificial Sequence
  Chemically synthetized sequence
ctcccaatagagcatctatt

SEQ ID NO: 728 - DNA, 20 bp, Artificial Sequence
  Chemically synthetized sequence
tcgttcaacaaaaaggtccc

SEQ ID NO: 729 - DNA, 20 bp, Artificial Sequence
  Chemically synthetized sequence
aacagttgctgtaggctttg

SEQ ID NO: 730 - DNA, 20 bp, Artificial Sequence
  Chemically synthetized sequence
taatccggcacatcataagg

SEQ ID NO: 731 - DNA, 20 bp, Artificial Sequence
  Chemically synthetized sequence
atgaggcaactagtgaccta

SEQ ID NO: 732 - DNA, 20 bp, Artificial Sequence
  Chemically synthetized sequence
ttgttccgttttgagtgact

SEQ ID NO: 733 - DNA, 20 bp, Artificial Sequence
  Chemically synthetized sequence
agatctccttatgcaagcag

SEQ ID NO: 734 - DNA, 20 bp, Artificial Sequence
  Chemically synthetized sequence
gttcaatgctgggtatttga

SEQ ID NO: 735 - DNA, 20 bp, Artificial Sequence
  Chemically synthetized sequence
gttcattgtttggcatagtc

SEQ ID NO: 736 - DNA, 20 bp, Artificial Sequence
  Chemically synthetized sequence
ccccaaatgtacaatttgtc

SEQ ID NO: 737 - DNA, 20 bp, Artificial Sequence
  Chemically synthetized sequence
aggaagatttggtccttgtc

SEQ ID NO: 738 - DNA, 20 bp, Artificial Sequence
  Chemically synthetized sequence
gattcttcctgatggttgag

SEQ ID NO: 739 - DNA, 20 bp, Artificial Sequence
  Chemically synthetized sequence
ggcttcttttggtagatact

SEQ ID NO: 740 - DNA, 20 bp, Artificial Sequence
  Chemically synthetized sequence
cgatattcgggattacagct

SEQ ID NO: 741 - DNA, 20 bp, Artificial Sequence
  Chemically synthetized sequence
attccttattctgggtctag

SEQ ID NO: 742 - DNA, 20 bp, Artificial Sequence
  Chemically synthetized sequence
agatgcttattctgctaggg

SEQ ID NO: 743 - DNA, 20 bp, Artificial Sequence
  Chemically synthetized sequence
aaaagtatgtctcccggttt

SEQ ID NO: 744 - DNA, 20 bp, Artificial Sequence
  Chemically synthetized sequence
caattagattccctgtgctg

SEQ ID NO: 745 - DNA, 20 bp, Artificial Sequence
  Chemically synthetized sequence
attttgaagtaacccctagg

SEQ ID NO: 746 - DNA, 20 bp, Artificial Sequence
  Chemically synthetized sequence
tattgagcttttcccacttc

SEQ ID NO: 747 - DNA, 20 bp, Artificial Sequence
  Chemically synthetized sequence
caatgggtgcatctgatctc

SEQ ID NO: 748 - DNA, 20 bp, Artificial Sequence
  Chemically synthetized sequence
tggagtgatgcattcagact

SEQ ID NO: 749 - DNA, 20 bp, Artificial Sequence
  Chemically synthetized sequence
cgtatgtgatcctgtttaca

SEQ ID NO: 750 - DNA, 20 bp, Artificial Sequence
  Chemically synthetized sequence
ttaacatatctgggacaggc

SEQ ID NO: 751 - DNA, 20 bp, Artificial Sequence
  Chemically synthetized sequence
ctgttgccaatttcagagtg

SEQ ID NO: 752 - DNA, 20 bp, Artificial Sequence
  Chemically synthetized sequence
tctagtttgtttctctggta

SEQ ID NO: 753 - DNA, 20 bp, Artificial Sequence
  Chemically synthetized sequence
cattttctatgaaacccgct

SEQ ID NO: 754 - DNA, 20 bp, Artificial Sequence
  Chemically synthetized sequence
ttccctcagaattttgatgc

SEQ ID NO: 755 - DNA, 20 bp, Artificial Sequence
  Chemically synthetized sequence
cttttgagatctgctgcttg

SEQ ID NO: 756 - DNA, 20 bp, Artificial Sequence
  Chemically synthetized sequence
tttgatcgattgctgcttga

SEQ ID NO: 757 - DNA, 20 bp, Artificial Sequence
  Chemically synthetized sequence
gatcaatcgattcagcttcc

SEQ ID NO: 758 - DNA, 20 bp, Artificial Sequence
  Chemically synthetized sequence
ggaatttctcgttggttttc

SEQ ID NO: 759 - DNA, 20 bp, Artificial Sequence
  Chemically synthetized sequence
cccttctacttctgagaatt

SEQ ID NO: 760 - DNA, 20 bp, Artificial Sequence
  Chemically synthetized sequence
catatttctcaaggtcctga

SEQ ID NO: 761 - DNA, 20 bp, Artificial Sequence
  Chemically synthetized sequence
gagatctattttagtgtcct

SEQ ID NO: 762 - DNA, 20 bp, Artificial Sequence
  Chemically synthetized sequence
aagaagctccgcgttgtatg

SEQ ID NO: 763 - DNA, 20 bp, Artificial Sequence
  Chemically synthetized sequence
attgtatgttggttctccag

SEQ ID NO: 764 - DNA, 20 bp, Artificial Sequence
  Chemically synthetized sequence
gtttgttcatttctgagtca

SEQ ID NO: 765 - DNA, 20 bp, Artificial Sequence
  Chemically synthetized sequence
gcttcatctctgtatacatc

SEQ ID NO: 766 - DNA, 20 bp, Artificial Sequence
  Chemically synthetized sequence
gatctggaaccggttgttta

SEQ ID NO: 767 - DNA, 20 bp, Artificial Sequence
  Chemically synthetized sequence
aatctttgtaccctgacttc

SEQ ID NO: 768 - DNA, 20 bp, Artificial Sequence
  Chemically synthetized sequence
cacatgatgaaccccaacaa

SEQ ID NO: 769 - DNA, 20 bp, Artificial Sequence
  Chemically synthetized sequence
aatgttgcacctaatgttgc

SEQ ID NO: 770 - DNA, 20 bp, Artificial Sequence
  Chemically synthetized sequence
gttactaccatgagtagtac

SEQ ID NO: 771 - DNA, 20 bp, Artificial Sequence
  Chemically synthetized sequence
tgcagattcgatctgcattg

SEQ ID NO: 772 - DNA, 20 bp, Artificial Sequence
  Chemically synthetized sequence
gtggtgagtttgacgatgtt

SEQ ID NO: 773 - DNA, 20 bp, Artificial Sequence
  Chemically synthetized sequence
ccttgagtagcagttttgac

SEQ ID NO: 774 - DNA, 20 bp, Artificial Sequence
  Chemically synthetized sequence
gtttctgttcctttgagatt

SEQ ID NO: 775 - DNA, 20 bp, Artificial Sequence
  Chemically synthetized sequence
ttgaggcattttgggcatag

SEQ ID NO: 776 - DNA, 20 bp, Artificial Sequence
  Chemically synthetized sequence
caaggctacgtccagatctg

SEQ ID NO: 777 - DNA, 20 bp, Artificial Sequence
  Chemically synthetized sequence
aaactcttgccgagggtatt

SEQ ID NO: 778 - DNA, 20 bp, Artificial Sequence
  Chemically synthetized sequence
ggtctgacttcatggagtat

SEQ ID NO: 779 - DNA, 20 bp, Artificial Sequence
  Chemically synthetized sequence
taggaaagcacccagatgta

SEQ ID NO: 780 - DNA, 20 bp, Artificial Sequence
  Chemically synthetized sequence
atttttgttctgtcgtgcat

SEQ ID NO: 781 - DNA, 20 bp, Artificial Sequence
  Chemically synthetized sequence
gtatcctcggagaaggttag

SEQ ID NO: 782 - DNA, 20 bp, Artificial Sequence
  Chemically synthetized sequence
acgttatgggttgataacct

SEQ ID NO: 783 - DNA, 20 bp, Artificial Sequence
  Chemically synthetized sequence
tggtgcattttctgcattga

SEQ ID NO: 784 - DNA, 20 bp, Artificial Sequence
  Chemically synthetized sequence
gaggttccaattttgtaggg

SEQ ID NO: 785 - DNA, 20 bp, Artificial Sequence
  Chemically synthetized sequence
ggtaatgttagggcaagacc

SEQ ID NO: 786 - DNA, 20 bp, Artificial Sequence
  Chemically synthetized sequence
ccaagccattgttgcgaaaa

SEQ ID NO: 787 - DNA, 20 bp, Artificial Sequence
  Chemically synthetized sequence
aacccccaaacggtaatttg

SEQ ID NO: 788 - DNA, 20 bp, Artificial Sequence
  Chemically synthetized sequence
catagagctttgccatttgg

SEQ ID NO: 789 - DNA, 20 bp, Artificial Sequence
  Chemically synthetized sequence
tggcagatgaggtgaacttc

SEQ ID NO: 790 - DNA, 20 bp, Artificial Sequence
  Chemically synthetized sequence
aacgtaatgtgtggtcactc

SEQ ID NO: 791 - DNA, 20 bp, Artificial Sequence
  Chemically synthetized sequence
caattctaccactttgtggt

SEQ ID NO: 792 - DNA, 20 bp, Artificial Sequence
  Chemically synthetized sequence
tgttttcccagatttttgca

SEQ ID NO: 793 - DNA, 20 bp, Artificial Sequence
  Chemically synthetized sequence
acccctttgataggtaattg

SEQ ID NO: 794 - DNA, 20 bp, Artificial Sequence
  Chemically synthetized sequence
ccacaccttttgaggcaata

SEQ ID NO: 795 - DNA, 20 bp, Artificial Sequence
  Chemically synthetized sequence
cttttattaccttgctcctg

SEQ ID NO: 796 - DNA, 20 bp, Artificial Sequence
  Chemically synthetized sequence
tttttcgtggaggcaatctg

SEQ ID NO: 797 - DNA, 20 bp, Artificial Sequence
  Chemically synthetized sequence
ttgcttttgtttaatccacc

SEQ ID NO: 798 - DNA, 20 bp, Artificial Sequence
  Chemically synthetized sequence
attgggcaatttcctatggc

SEQ ID NO: 799 - DNA, 20 bp, Artificial Sequence
  Chemically synthetized sequence
agcttcaagggtgttttcac

SEQ ID NO: 800 - DNA, 20 bp, Artificial Sequence
  Chemically synthetized sequence
atagttttgcaggaggtcta

SEQ ID NO: 801 - DNA, 20 bp, Artificial Sequence
  Chemically synthetized sequence
ttgttatcttgtttatggcc

SEQ ID NO: 802 - DNA, 20 bp, Artificial Sequence
  Chemically synthetized sequence
ccagctcactcaaagagttg

SEQ ID NO: 803 - DNA, 20 bp, Artificial Sequence
  Chemically synthetized sequence
accgcttagtctttgaagat

SEQ ID NO: 804 - DNA, 20 bp, Artificial Sequence
  Chemically synthetized sequence
atttcgttgtggagttcatc

SEQ ID NO: 805 - DNA, 20 bp, Artificial Sequence
  Chemically synthetized sequence
cactttctcatctagttcta

SEQ ID NO: 806 - DNA, 20 bp, Artificial Sequence
  Chemically synthetized sequence
ttgtatcagctctgagatca

SEQ ID NO: 807 - DNA, 20 bp, Artificial Sequence
  Chemically synthetized sequence
tgcgagttctatttgtgagc

SEQ ID NO: 808 - DNA, 20 bp, Artificial Sequence
  Chemically synthetized sequence
ttccttcattggaaagcagg

SEQ ID NO: 809 - DNA, 20 bp, Artificial Sequence
  Chemically synthetized sequence
gccaagagatgttcatcttc

SEQ ID NO: 810 - DNA, 20 bp, Artificial Sequence
  Chemically synthetized sequence
cagcattttcttcagctttc

SEQ ID NO: 811 - DNA, 20 bp, Artificial Sequence
  Chemically synthetized sequence
cagctattctgtcgagacag

SEQ ID NO: 812 - DNA, 20 bp, Artificial Sequence
  Chemically synthetized sequence
cagtgaatcaaaggtgggga

SEQ ID NO: 813 - DNA, 20 bp, Artificial Sequence
  Chemically synthetized sequence
ttatccaatccatcgtcatt

SEQ ID NO: 814 - DNA, 20 bp, Artificial Sequence
  Chemically synthetized sequence
aggcagcagttgagtagtaa

SEQ ID NO: 815 - DNA, 20 bp, Artificial Sequence
  Chemically synthetized sequence
atcatcagtgttacagccaa

SEQ ID NO: 816 - DNA, 20 bp, Artificial Sequence
  Chemically synthetized sequence
attgtctctggagaccatat

SEQ ID NO: 817 - DNA, 20 bp, Artificial Sequence
  Chemically synthetized sequence
cttcccttatagacagatgg

SEQ ID NO: 818 - DNA, 20 bp, Artificial Sequence
  Chemically synthetized sequence
tgggtctctacagtgtagaa

SEQ ID NO: 819 - DNA, 20 bp, Artificial Sequence
  Chemically synthetized sequence
aatcagtttctggatgtgct

SEQ ID NO: 820 - DNA, 20 bp, Artificial Sequence
  Chemically synthetized sequence
agttcattggtactggtagt

SEQ ID NO: 821 - DNA, 20 bp, Artificial Sequence
  Chemically synthetized sequence
ccagattttggcgatctatt

SEQ ID NO: 822 - DNA, 20 bp, Artificial Sequence
  Chemically synthetized sequence
ggggtaaagctggaatcaac

SEQ ID NO: 823 - DNA, 20 bp, Artificial Sequence
  Chemically synthetized sequence
gggacttatgactacccaaa

SEQ ID NO: 824 - DNA, 20 bp, Artificial Sequence
  Chemically synthetized sequence
atgcgataacacgtgcatgg

SEQ ID NO: 825 - DNA, 20 bp, Artificial Sequence
  Chemically synthetized sequence
gctgctttgaattttaccac

SEQ ID NO: 826 - DNA, 20 bp, Artificial Sequence
  Chemically synthetized sequence
acaatgccaaggaaattgga

SEQ ID NO: 827 - DNA, 20 bp, Artificial Sequence
  Chemically synthetized sequence
ctaccacgattcaaatgtga

SEQ ID NO: 828 - DNA, 20 bp, Artificial Sequence
  Chemically synthetized sequence
tacaatgccgaactgttggt

SEQ ID NO: 829 - DNA, 20 bp, Artificial Sequence
  Chemically synthetized sequence
ggtaaagagttcaaccacct

SEQ ID NO: 830 - DNA, 20 bp, Artificial Sequence
  Chemically synthetized sequence
cacacagaatgccattgacg

SEQ ID NO: 831 - DNA, 20 bp, Artificial Sequence
  Chemically synthetized sequence
gatatgcagccgacctgaag

SEQ ID NO: 832 - DNA, 20 bp, Artificial Sequence
  Chemically synthetized sequence
accatcaaaatgagcagggg

SEQ ID NO: 833 - DNA, 20 bp, Artificial Sequence
  Chemically synthetized sequence
tctattcaatctagaggcct

SEQ ID NO: 834 - DNA, 20 bp, Artificial Sequence
  Chemically synthetized sequence
aaattgagactggccacagg

SEQ ID NO: 835 - DNA, 20 bp, Artificial Sequence
  Chemically synthetized sequence
gtgctataaacaccagcctc

SEQ ID NO: 836 - DNA, 20 bp, Artificial Sequence
  Chemically synthetized sequence
tacaacttgtcaaacaccca

SEQ ID NO: 837 - DNA, 20 bp, Artificial Sequence
  Chemically synthetized sequence
agatacaccagtccacgatt

SEQ ID NO: 838 - DNA, 20 bp, Artificial Sequence
  Chemically synthetized sequence
tgctggatctggtattatca

SEQ ID NO: 839 - DNA, 20 bp, Artificial Sequence
  Chemically synthetized sequence
agatatgcattcgcaatgga

SEQ ID NO: 840 - DNA, 20 bp, Artificial Sequence
  Chemically synthetized sequence
gcaactggaaatctagtggt

SEQ ID NO: 841 - DNA, 20 bp, Artificial Sequence
  Chemically synthetized sequence
gccgggagacaaaataacat

SEQ ID NO: 842 - DNA, 20 bp, Artificial Sequence
  Chemically synthetized sequence
actattactggacactagta

SEQ ID NO: 843 - DNA, 20 bp, Artificial Sequence
  Chemically synthetized sequence
cccaaagtgagggatcaaga

SEQ ID NO: 844 - DNA, 20 bp, Artificial Sequence
  Chemically synthetized sequence
agttcaagccggaaatagca

SEQ ID NO: 845 - DNA, 20 bp, Artificial Sequence
  Chemically synthetized sequence
gggtcatcaagatacagcaa

SEQ ID NO: 846 - DNA, 20 bp, Artificial Sequence
  Chemically synthetized sequence
tgcagatgcatatgtttttg

SEQ ID NO: 847 - DNA, 20 bp, Artificial Sequence
  Chemically synthetized sequence
gctgaccaacaaagtctcta

SEQ ID NO: 848 - DNA, 20 bp, Artificial Sequence
  Chemically synthetized sequence
cattcaccatccatctacta

SEQ ID NO: 849 - DNA, 20 bp, Artificial Sequence
  Chemically synthetized sequence
aagaagtcctcgtgctatgg

SEQ ID NO: 850 - DNA, 20 bp, Artificial Sequence
  Chemically synthetized sequence
agctcagcaaatcctacatt

SEQ ID NO: 851 - DNA, 20 bp, Artificial Sequence
  Chemically synthetized sequence
aaggtgtaacggcagcatgt

SEQ ID NO: 852 - DNA, 20 bp, Artificial Sequence
  Chemically synthetized sequence
tggcccaatcatgactcgaa

SEQ ID NO: 853 - DNA, 20 bp, Artificial Sequence
  Chemically synthetized sequence
gatattccccaagacaagtt

SEQ ID NO: 854 - DNA, 20 bp, Artificial Sequence
  Chemically synthetized sequence
tatgaggagctaagagagca

SEQ ID NO: 855 - DNA, 20 bp, Artificial Sequence
  Chemically synthetized sequence
tgttacccaggagatttcat

SEQ ID NO: 856 - DNA, 20 bp, Artificial Sequence
  Chemically synthetized sequence
cacctagttcagacaatgga

SEQ ID NO: 857 - DNA, 20 bp, Artificial Sequence
  Chemically synthetized sequence
tcatggtcctacattgtgga

SEQ ID NO: 858 - DNA, 20 bp, Artificial Sequence
  Chemically synthetized sequence
tgaatcactctccacagcaa

SEQ ID NO: 859 - DNA, 20 bp, Artificial Sequence
  Chemically synthetized sequence
cccattgcatttgggtaaat

SEQ ID NO: 860 - DNA, 20 bp, Artificial Sequence
  Chemically synthetized sequence
gcataacgggaaactatgca

SEQ ID NO: 861 - DNA, 20 bp, Artificial Sequence
  Chemically synthetized sequence
acacactctgttaaccttct

SEQ ID NO: 862 - DNA, 20 bp, Artificial Sequence
  Chemically synthetized sequence
acagacactgtagacacagt

SEQ ID NO: 863 - DNA, 20 bp, Artificial Sequence
  Chemically synthetized sequence
atgtataggttatcatgcga

SEQ ID NO: 864 - DNA, 20 bp, Artificial Sequence
  Chemically synthetized sequence
caaccgcaaatgcagacaca

SEQ ID NO: 865 - DNA, 20 bp, Artificial Sequence
  Chemically synthetized sequence
ggcaatactagtagttctgc

SEQ ID NO: 866 - DNA, 20 bp, Artificial Sequence
  Chemically synthetized sequence
ggcaacattaggtgcaacat

SEQ ID NO: 867 - DNA, 20 bp, Artificial Sequence
  Chemically synthetized sequence
ttttgctttgtgttgctttg

SEQ ID NO: 868 - DNA, 20 bp, Artificial Sequence
  Chemically synthetized sequence
atttcctttgccatatcatg

SEQ ID NO: 869 - DNA, 20 bp, Artificial Sequence
  Chemically synthetized sequence
gggtacaaagattggatcct

SEQ ID NO: 870 - DNA, 20 bp, Artificial Sequence
  Chemically synthetized sequence
caagggagttgagctgaagt

SEQ ID NO: 871 - DNA, 20 bp, Artificial Sequence
  Chemically synthetized sequence
agcattaaacaaccggttcc

SEQ ID NO: 872 - DNA, 20 bp, Artificial Sequence
  Chemically synthetized sequence
atgctgaggatatgggcaat

SEQ ID NO: 873 - DNA, 20 bp, Artificial Sequence
  Chemically synthetized sequence
ccctggagaaccaacataca

SEQ ID NO: 874 - DNA, 20 bp, Artificial Sequence
  Chemically synthetized sequence
gatctctggtcatacaacgc

SEQ ID NO: 875 - DNA, 20 bp, Artificial Sequence
  Chemically synthetized sequence
gaagggagaattcaggacct

SEQ ID NO: 876 - DNA, 20 bp, Artificial Sequence
  Chemically synthetized sequence
accaacgagaaattccatca

SEQ ID NO: 877 - DNA, 20 bp, Artificial Sequence
  Chemically synthetized sequence
gctgaatcgattgatcggga

SEQ ID NO: 878 - DNA, 20 bp, Artificial Sequence
  Chemically synthetized sequence
aagcactcaagcagcaatcg

SEQ ID NO: 879 - DNA, 20 bp, Artificial Sequence
  Chemically synthetized sequence
ctgagggaagaggacaagca

SEQ ID NO: 880 - DNA, 20 bp, Artificial Sequence
  Chemically synthetized sequence
atggttggtacggtttcagg

SEQ ID NO: 881 - DNA, 20 bp, Artificial Sequence
  Chemically synthetized sequence
aatggttgggagggaatggt

SEQ ID NO: 882 - DNA, 20 bp, Artificial Sequence
  Chemically synthetized sequence
atgcggaatgtaccagagaa

SEQ ID NO: 883 - DNA, 20 bp, Artificial Sequence
  Chemically synthetized sequence
gcactctgaaattggcaaca

SEQ ID NO: 884 - DNA, 20 bp, Artificial Sequence
  Chemically synthetized sequence
tgtcccagatatgttaagca

SEQ ID NO: 885 - DNA, 20 bp, Artificial Sequence
  Chemically synthetized sequence
aaacaggatcacatacgggg

SEQ ID NO: 886 - DNA, 20 bp, Artificial Sequence
  Chemically synthetized sequence
gcaagtctgaatgcatcact

SEQ ID NO: 887 - DNA, 20 bp, Artificial Sequence
  Chemically synthetized sequence
taatgagatcagatgcaccc

SEQ ID NO: 888 - DNA, 20 bp, Artificial Sequence
  Chemically synthetized sequence
tcctaggggttacttcaaaa

SEQ ID NO: 889 - DNA, 20 bp, Artificial Sequence
  Chemically synthetized sequence
acagcacagggaatctaatt

SEQ ID NO: 890 - DNA, 20 bp, Artificial Sequence
  Chemically synthetized sequence
ccgggagacatacttttgat

SEQ ID NO: 891 - DNA, 20 bp, Artificial Sequence
  Chemically synthetized sequence
agcatctattggacaatagt

SEQ ID NO: 892 - DNA, 20 bp, Artificial Sequence
  Chemically synthetized sequence
taaggaatatccctagcaga

SEQ ID NO: 893 - DNA, 20 bp, Artificial Sequence
  Chemically synthetized sequence
aatatcggatctagacccag

SEQ ID NO: 894 - DNA, 20 bp, Artificial Sequence
  Chemically synthetized sequence
aagccaacaagctgtaatcc

SEQ ID NO: 895 - DNA, 20 bp, Artificial Sequence
  Chemically synthetized sequence
atgctcaaccatcaggaaga

SEQ ID NO: 896 - DNA, 20 bp, Artificial Sequence
  Chemically synthetized sequence
ggacaaggaccaaatcttcc

SEQ ID NO: 897 - DNA, 20 bp, Artificial Sequence
  Chemically synthetized sequence
aattgtacatttggggggtt

SEQ ID NO: 898 - DNA, 20 bp, Artificial Sequence
  Chemically synthetized sequence
cattgaacgtgactatgcca

SEQ ID NO: 899 - DNA, 20 bp, Artificial Sequence
  Chemically synthetized sequence
ggttgacccacttaaacttc

SEQ ID NO: 900 - DNA, 20 bp, Artificial Sequence
  Chemically synthetized sequence
ctgcttgcataaggagatct

SEQ ID NO: 901 - DNA, 20 bp, Artificial Sequence
  Chemically synthetized sequence
ggagtcactcaaaacggaac

SEQ ID NO: 902 - DNA, 20 bp, Artificial Sequence
  Chemically synthetized sequence
tccggcacactggagtttaa

SEQ ID NO: 903 - DNA, 20 bp, Artificial Sequence
  Chemically synthetized sequence
ctcccttaggtcactagttg

SEQ ID NO: 904 - DNA, 20 bp, Artificial Sequence
  Chemically synthetized sequence
cttatgatgtgccggattat

SEQ ID NO: 905 - DNA, 20 bp, Artificial Sequence
  Chemically synthetized sequence
caaagcctacagcaactgtt

SEQ ID NO: 906 - DNA, 20 bp, Artificial Sequence
  Chemically synthetized sequence
gggacctttttgttgaacga

SEQ ID NO: 907 - DNA, 20 bp, Artificial Sequence
  Chemically synthetized sequence
tagatgctctattgggagac

SEQ ID NO: 908 - DNA, 20 bp, Artificial Sequence
  Chemically synthetized sequence
ggtgaaatatgcgacagtcc

SEQ ID NO: 909 - DNA, 20 bp, Artificial Sequence
  Chemically synthetized sequence
atcacgaatgaccgaattga

SEQ ID NO: 910 - DNA, 20 bp, Artificial Sequence
  Chemically synthetized sequence
tgcagtaccaaacggaacga

SEQ ID NO: 911 - DNA, 20 bp, Artificial Sequence
  Chemically synthetized sequence
tgacaatagcacggcaacgc

SEQ ID NO: 912 - DNA, 20 bp, Artificial Sequence
  Chemically synthetized sequence
atgtctggttttcgctcaaa

SEQ ID NO: 913 - DNA, 20 bp, Artificial Sequence
  Chemically synthetized sequence
gactatcattgctttgagct

SEQ ID NO: 914 - DNA, 20 bp, Artificial Sequence
  Chemically synthetized sequence
ctataagggaagttaagccc

SEQ ID NO: 915 - DNA, 20 bp, Artificial Sequence
  Chemically synthetized sequence
acaatgtttcttgctccatc

SEQ ID NO: 916 - DNA, 20 bp, Artificial Sequence
  Chemically synthetized sequence
tgttgtttatatggtctcca

SEQ ID NO: 917 - DNA, 20 bp, Artificial Sequence
  Chemically synthetized sequence
cagtttggctgtaacactga

SEQ ID NO: 918 - DNA, 20 bp, Artificial Sequence
  Chemically synthetized sequence
ctttactactcaactgctgc

SEQ ID NO: 919 - DNA, 20 bp, Artificial Sequence
  Chemically synthetized sequence
ccacctttgattcactgaat

SEQ ID NO: 920 - DNA, 20 bp, Artificial Sequence
  Chemically synthetized sequence
gacagaatagctgctggtac

SEQ ID NO: 921 - DNA, 20 bp, Artificial Sequence
  Chemically synthetized sequence
cacaagtgcaaccagacctg

SEQ ID NO: 922 - DNA, 20 bp, Artificial Sequence
  Chemically synthetized sequence
agctgaagaaaatgctgggc

SEQ ID NO: 923 - DNA, 20 bp, Artificial Sequence
  Chemically synthetized sequence
catctcttggcgcttgaaag

SEQ ID NO: 924 - DNA, 20 bp, Artificial Sequence
  Chemically synthetized sequence
cctgctttccaatgaaggaa

SEQ ID NO: 925 - DNA, 20 bp, Artificial Sequence
  Chemically synthetized sequence
gctcacaaatagaactcgca

SEQ ID NO: 926 - DNA, 20 bp, Artificial Sequence
  Chemically synthetized sequence
agtggatgatctcagagctg

SEQ ID NO: 927 - DNA, 20 bp, Artificial Sequence
  Chemically synthetized sequence
ccatggatgaactccacaac

SEQ ID NO: 928 - DNA, 20 bp, Artificial Sequence
  Chemically synthetized sequence
ctcaactctttgagtgagct

SEQ ID NO: 929 - DNA, 20 bp, Artificial Sequence
  Chemically synthetized sequence
gcactcaagaggccataaac

SEQ ID NO: 930 - DNA, 20 bp, Artificial Sequence
  Chemically synthetized sequence
aggaatgattgcaggttggc

SEQ ID NO: 931 - DNA, 20 bp, Artificial Sequence
  Chemically synthetized sequence
tagacctcctgcaaaactat

SEQ ID NO: 932 - DNA, 20 bp, Artificial Sequence
  Chemically synthetized sequence
ttgaagctggccaatggaac

SEQ ID NO: 933 - DNA, 20 bp, Artificial Sequence
  Chemically synthetized sequence
gcccaatatgggtgaaaaca

SEQ ID NO: 934 - DNA, 20 bp, Artificial Sequence
  Chemically synthetized sequence
acatgcaaaggccataggaa

SEQ ID NO: 935 - DNA, 20 bp, Artificial Sequence
  Chemically synthetized sequence
aagcaagccttactacacag

SEQ ID NO: 936 - DNA, 20 bp, Artificial Sequence
  Chemically synthetized sequence
tgcctccacgaaaaatacgg

SEQ ID NO: 937 - DNA, 20 bp, Artificial Sequence
  Chemically synthetized sequence
aagtggcaggagcaaggtaa

SEQ ID NO: 938 - DNA, 20 bp, Artificial Sequence
  Chemically synthetized sequence
aaggggtattttattgcctc

SEQ ID NO: 939 - DNA, 20 bp, Artificial Sequence
  Chemically synthetized sequence
gttgttgattacatggtgca

SEQ ID NO: 940 - DNA, 20 bp, Artificial Sequence
  Chemically synthetized sequence
acggaggactaccacaaagt

SEQ ID NO: 941 - DNA, 20 bp, Artificial Sequence
  Chemically synthetized sequence
gtgaccacacattacgtttc

SEQ ID NO: 942 - DNA, 20 bp, Artificial Sequence
  Chemically synthetized sequence
cagaagttcacctcatctgc

SEQ ID NO: 943 - DNA, 20 bp, Artificial Sequence
  Chemically synthetized sequence
agctctatggggactcaaag

SEQ ID NO: 944 - DNA, 20 bp, Artificial Sequence
  Chemically synthetized sequence
tgacgacgagacccaaatgg

SEQ ID NO: 945 - DNA, 20 bp, Artificial Sequence
  Chemically synthetized sequence
aaattaccgtttgggggttc

SEQ ID NO: 946 - DNA, 20 bp, Artificial Sequence
  Chemically synthetized sequence
ttttcgcaacaatggcttgg

SEQ ID NO: 947 - DNA, 20 bp, Artificial Sequence
  Chemically synthetized sequence
ccctaacattaccaatggaa

SEQ ID NO: 948 - DNA, 20 bp, Artificial Sequence
  Chemically synthetized sequence
aaattggaacctcagggtct

SEQ ID NO: 949 - DNA, 20 bp, Artificial Sequence
  Chemically synthetized sequence
gcagaaaatgcaccaggagg

SEQ ID NO: 950 - DNA, 20 bp, Artificial Sequence
  Chemically synthetized sequence
ggttatcaacccataacgtt

SEQ ID NO: 951 - DNA, 20 bp, Artificial Sequence
  Chemically synthetized sequence
cttctccgaggatacgaaca

SEQ ID NO: 952 - DNA, 20 bp, Artificial Sequence
  Chemically synthetized sequence
tgctttcctataatgcacga

SEQ ID NO: 953 - DNA, 20 bp, Artificial Sequence
  Chemically synthetized sequence
agtcagacctgttacatctg

SEQ ID NO: 954 - DNA, 20 bp, Artificial Sequence
  Chemically synthetized sequence
cctcggcaagagtttcaata

SEQ ID NO: 955 - DNA, 20 bp, Artificial Sequence
  Chemically synthetized sequence
ttgggcagaccaaaatgcac

SEQ ID NO: 956 - DNA, 20 bp, Artificial Sequence
  Chemically synthetized sequence
aaatgcctcaactgcacaga

SEQ ID NO: 957 - DNA, 20 bp, Artificial Sequence
  Chemically synthetized sequence
ggaacagaaaccagggggaa

SEQ ID NO: 958 - DNA, 20 bp, Artificial Sequence
  Chemically synthetized sequence
gtcgtcaaaactgctactca

SEQ ID NO: 959 - DNA, 20 bp, Artificial Sequence
  Chemically synthetized sequence
aacatcgtcaaactcaccac

SEQ ID NO: 960 - DNA, 20 bp, Artificial Sequence
  Chemically synthetized sequence
cagatcgaatctgcactggg

SEQ ID NO: 961 - DNA, 20 bp, Artificial Sequence
  Chemically synthetized sequence
actcatggtagtaacatcca